(19) United States
(12) Patent Application Publication
(10) Pub. No.: US 2022/0184129 A1
(43) Pub. Date: Jun. 16, 2022

(54) Compositions and Methods Comprising a High Affinity Chimeric Antigen Receptor (CAR) with Cross-Reactivity to Clinically-Relevant EGFR Mutated Proteins

(71) Applicant: THE TRUSTEES OF THE UNIVERSITY OF PENNSYLVANIA, Philadelphia, PA (US)

(72) Inventors: Donald O'ROURKE, Wynnewood, PA (US); Zev BINDER, Merion Station, PA (US); Michael MILONE, Cherry Hill, NJ (US); Radhika THOKALA, Conshohocken, PA (US)

(21) Appl. No.: 17/602,949

(22) PCT Filed: Apr. 11, 2020
(86) PCT No.: PCT/US2020/027859

§ 371 Date: Oct. 11, 2021

Related U.S. Application Data
(60) Provisional application No. 62/833,456, filed on Apr. 12, 2019.
(60) Provisional application No. 62/892,343, filed on Aug. 27, 2019.

(51) Int. Cl. A61K 35/17; A61K 39/00; A61P 35/00; C07K 14/725; C07K 16/28

(52) U.S. Cl. CPC A61K 35/17; A61K 39/001104; A61P 35/00; C07K 14/7051; C07K 16/2818; C07K 16/2863; C12N 2740/16043; A61K 2039/5156; C07K 2317/24; C07K 2317/33; C07K 2317/76; C07K 2319/03; A61K 2039/507

(57) ABSTRACT

The present invention includes compositions and methods that utilize a high affinity chimeric antigen receptor (CAR) with cross-reactivity to clinically-relevant EGFR mutated proteins.

### CROSS-REFERENCE TO RELATED APPLICATIONS

The present application claims priority under 35 U.S.C. § 119(e) to U.S. Provisional Application No. 62/833,456, filed Apr. 12, 2019, and U.S. Provisional Application No. 62/892,343, filed Aug. 27, 2019, each of which are hereby incorporated herein by reference in their entireties.

### BACKGROUND OF THE INVENTION

Glioblastoma (GBM), or glioma grade IV, is a devastating cancer with an annual incidence of 3.19/100,000 individuals per year (˜10,000) and a median survival of 14.6 months following standard-of-care surgery, radiotherapy, and chemotherapy. Few advances in treatment have been realized over the past 20 years, and 2-year survival remains close to 25%. While low intensity, alternating electric fields have recently shown some potential, there still remains a significant need for novel treatments.

Research over the past 20 years demonstrates that cancer is naturally recognized by the immune system and immune escape is a central part of oncogenesis. This is most pointedly illustrated by the dramatic clinical responses observed in cancers such as melanoma and non-small cell lung cancer (NSCLC) upon disruption of the natural immune checkpoints mediated by programmed death 1 (PD-1) and cytotoxic T lymphocyte attenuator 4 (CTLA-4). Even in cancers where natural immunity may be more limited, such as acute lymphocytic leukemia, synthetic chimeric antigen receptor (CAR)-based immunotherapies like tisagenlecleucel (KYMRIAH®) and axicabtagene ciloleucel (YESCARTA®) demonstrate the immense cytotoxic potential of T cells to produce durable tumor control.

GBM has a paucity of neoantigens, making T cell receptor (TCR)-based immunotherapy challenging. Based upon The Cancer Genome Atlas (TCGA) whole exome sequencing data, the mutational load within GBM is moderate when compared to other tumor types. However, the median GBM mutational frequency is 1-2 orders of magnitude lower relative to immunogenic tumors such as melanoma, NSCLC and bladder cancer, and available evidence supports mutational burden as one of the best correlates of response to immune checkpoint blockade (ICB). The low mutational burden and poor T cell infiltration, combined with the challenges of delivering large molecules like antibodies across the blood-brain barrier (BBB), may explain the generally poor observed responses to PD-1 inhibitor therapy in GBM to date.

Alterations within the epidermal growth factor receptor (EGFR) (ErbB1) locus represent the most frequent genetic alteration in GBM. EGFR overexpression, such as that mediated through focal amplification of the EGFR locus as double minute chromosomes, has long been recognized in GBM, and is found in 30% of cases. EGFR mutations are also frequent. The oncogenic EGFR variant lacking exons 2-7 (EGFRvIII) is found in approximately 30% of GBM.

In-human clinical trials of autologous EGFRvIII-specific CART cell (CART-EGFRvIII) therapy in recurrent GBM have been completed. In addition to demonstrating safety, biopsy of specific regions of GBM following CART infusion established that CART-EGFRvIII cells are capable of penetrating the BBB, infiltrating GBM and mediating on-target activity. Immunohistochemical (IHC) analysis and RNA in situ hybridization (ISH) showed that tumor-infiltrating T cells displayed an activated phenotype, represented by an increase in CD8+ granzyme B+ CD25+ CART cells in regions of viable GBM tissue. These observations were observed between days 7-14 after CART infusion, consistent with the peripheral engraftment peak at 7-10 days that was seen across the spectrum of treated patients. Regionally-specific antigen editing and reduction of EGFRvIII was observed in 5 of 7 treated specimens, supportive of target-specific activity produced by therapy. Clinical activity of CART cells targeting HER-2 and IL-13Rα2 has also been reported in GBM.

Tumor heterogeneity and an immunosuppressive tumor microenvironment (TME) are major obstacles to CART therapy in GBM. The site-specific reduction in EGFRvIII following CART therapy is consistent with the observed intratumoral heterogeneity of this alteration in GBM. Importantly, IHC analysis of tissues demonstrated the presence of an adaptive immune response within the GBM TME that closely followed the temporal sequence of CART activation. IDOL PD-L1, IL10 and TGFβ were all increased within tumor tissue proximal to CART cells following treatment. These immunoregulatory pathways have known roles in the evasion of tumor immunity, and are consistent with the emergence of adaptive resistance that can further blunt anti-tumor activity of CART cells against GBM.

There remains a need for novel CART therapies that target multiple antigens as well as circumvent immunosuppressive signals within the GBM TME. The present invention addresses this need.

### SUMMARY OF THE INVENTION

The present invention is based on the discovery of high affinity chimeric antigen receptors (CARs) with cross-reactivity to clinically-relevant EGFR mutated proteins.

In one aspect, the invention provides an isolated nucleic acid molecule encoding a chimeric antigen receptor (CAR), wherein the CAR comprises an antigen binding domain capable of binding multiple isoforms of epidermal growth factor receptor (EGFR), a transmembrane domain, and an intracellular domain.

In another aspect, the invention provides a vector comprising any of the isolated nucleic acids disclosed herein.

In another aspect, the invention provides a modified cell comprising a cross-reactive chimeric antigen receptor (CAR), wherein the CAR comprises an antigen binding domain capable of binding multiple isoforms of EGFR, a transmembrane domain, and an intracellular domain.

In another aspect, the invention provides a method for treating cancer in a subject in need thereof. The method comprises administering to the subject any of the modified cells disclosed herein.

In another aspect, the invention provides a method for treating cancer in a subject in need thereof. The method comprises administering to the subject a modified cell comprising a CAR. The CAR comprises an antigen binding domain capable of binding multiple isoforms of EGFR, a transmembrane domain, and an intracellular domain.

In various embodiments of the above aspects or any other aspect of the invention delineated herein, the EGFR isoforms are selected from the group consisting of wild-type EGFR (wtEGFR), mutated EGFR, EGFRA289V, EGFRA289D, EGFRA289T, EGFRR108K, EGFRR108G, EGFRG598V, EGFRD126Y, EGFRC628F, EGFRR108K/A289V, EGFRR108K/D126Y, EGFRA289V/G598V, EGFRA289V/C628F, and EGFR variant II.

In certain exemplary embodiments, the antigen binding domain is selected from the group consisting of an antibody, an scFv, a Fab, or any fragment thereof.

In certain exemplary embodiments, the antigen binding domain is encoded by a nucleotide sequence selected from the group consisting of SEQ ID NO: 1, SEQ ID NO: 31, SEQ ID NO: 79, SEQ ID NO: 81, SEQ ID NO: 83, and SEQ ID NO: 85. In certain exemplary embodiments, the antigen binding domain comprises an amino acid sequence selected from the group consisting of SEQ ID NO: 2, SEQ ID NO: 32, SEQ ID NO: 80, SEQ ID NO: 82, SEQ ID NO: 84, and SEQ ID NO: 86.

In certain exemplary embodiments, the antigen binding domain comprises a light chain variable region comprising an amino acid sequence selected from the group consisting of SEQ ID NO: 3, SEQ ID NO: 27, and SEQ ID NO: 30. In certain exemplary embodiments, the antigen binding domain comprises a heavy chain variable region comprising an amino acid sequence selected from the group consisting of SEQ ID NO: 4, SEQ ID NO: 26 and SEQ ID NO: 29.

In certain exemplary embodiments, the antigen binding domain comprises a light chain complementarity determining region (LCDR) comprising an amino acid sequence selected from the group consisting of SEQ ID NOs: 5, 6, and 7. In certain exemplary embodiments, the antigen binding domain comprises a heavy chain complementarity determining region (HCDR) comprising an amino acid sequence selected from the group consisting of SEQ ID NOs: 8, 9, and 10 .

In certain exemplary embodiments, the CAR further comprises a hinge region. In certain exemplary embodiments, the hinge region is encoded by the nucleotide sequence of SEQ ID NO: 11 or SEQ ID NO: 71. In certain exemplary embodiments, the hinge region comprises the amino acid sequence of SEQ ID NO: 72.

In certain exemplary embodiments, the transmembrane domain is encoded by the nucleotide sequence of SEQ ID NO: 12 or SEQ ID NO: 73. In certain exemplary embodiments, the transmembrane domain comprises the amino acid sequence of SEQ ID NO: 74.

In certain exemplary embodiments, the intracellular domain is encoded by the nucleotide sequence of SEQ ID NO: 13 or SEQ ID NO: 75. In certain exemplary embodiments, the intracellular domain is encoded by the nucleotide sequence comprising SEQ ID NO: 14 or SEQ ID NO: 77. In certain exemplary embodiments, the intracellular domain is encoded by a nucleotide sequence comprising SEQ ID NO: 13 and SEQ ID NO: 14 or a nucleotide sequence comprising SEQ ID NO: 75 and SEQ ID NO: 77. In certain exemplary embodiments, the intracellular domain comprises the amino acid sequence of SEQ ID NO: 76. In certain exemplary embodiments, the intracellular domain comprises the amino acid sequence of SEQ ID NO: 78. In certain exemplary embodiments, intracellular domain comprises the amino acid sequence of SEQ ID NO: 76 and SEQ ID NO: 78.

In certain exemplary embodiments, the CAR is encoded by a nucleotide sequence selected from the group consisting of SEQ ID NOs: 21, 64, 66, or 68. In certain exemplary embodiments, the CAR comprises an amino acid sequence selected from the group consisting of SEQ ID NOs: 22, 65, 67, and 69.

In certain exemplary embodiments, the transmembrane domain and/or the intracellular domain comprise a killer cell immunoglobulin-like receptor (KIR).

In certain exemplary embodiments, the nucleic acid further comprises a nucleic acid encoding DAP12.

In certain exemplary embodiments, the CAR is capable of binding an EGFR homodimer, an EGFR heterodimer, an EGFR oligomer, and/or an EGFR/ErbB oligomer.

In certain exemplary embodiments, the cell is a T cell. In certain exemplary embodiments, the cell is an autologous cell. In certain exemplary embodiments, the cell is a human cell.

In certain exemplary embodiments, the method further comprises administering an additional treatment to the subject. In certain exemplary embodiments, the additional treatment comprises an immune checkpoint blockade (ICB). In certain exemplary embodiments, the ICB is selected from the group consisting of an anti-PD-1 treatment, an anti-PD-L1 treatment, an anti-TIM3 treatment, and an anti CTLA-4 treatment.

In certain exemplary embodiments, the treatment is delivered locally.

In certain exemplary embodiments, the modified cell further comprises a minibody. In certain exemplary embodiments, the minibody comprises an scFv specific for PD-1 and a human IgG CH3 domain. In certain exemplary embodiments, the minibody comprises an scFv specific for CTLA-4 and a human IgG CH3 domain. In certain exemplary embodiments, the minibody comprises an scFv specific for TIM-3 and a human IgG CH3 domain. In certain exemplary embodiments, the minibody comprises an scFv specific for PD-L1 and a human IgG CH3 domain.

In another aspect, the invention provides a method of treating cancer in a subject in need thereof. The method comprises culturing a plurality of CAR T cells with a GBM organoid (GBO) derived from the subject, selecting from the plurality of CAR T cells, a CAR T cell having the highest efficacy, and administering the CAR T cell with the highest efficacy to the subject, thus treating the cancer in the subject. In certain exemplary embodiments, the plurality of CART cells comprises a plurality of modified T cells comprising a plurality of CARs, wherein each CAR comprises an antigen binding domain, a transmembrane domain, and an intracellular domain. In certain exemplary embodiments, the antigen binding domain is capable of binding an antigen selected from the group consisting of CD19, EGFR, multiple isoforms of EGFR (e.g. wild-type EGFR (wtEGFR), mutated EGFR, EGFRA289V, EGFRA289D, EGFRA289T, EGFRR108K, EGFRR108G, EGFRG598V, EGFRD126Y, EGFRC628F, EGFRR108K/A289V, EGFRR108K/D126Y, EGFRA289V/G598V, EGFRA289V/C628F, and EGFR variant II), PSMA, PSCA, and any tumor associated antigen (TAA).

In certain exemplary embodiments, the GBO is generated from a biopsy from the subject. the highest efficacy is measured as the highest degree of apoptosis and/or tumor cell killing.

In certain exemplary embodiments, the method further comprises administering an additional treatment to the subject. In certain exemplary embodiments, the additional treatment comprises an immune checkpoint blockade (ICB). In certain exemplary embodiments, the ICB is selected from the group consisting of an anti-PD-1 treatment, an anti-PD-L1 treatment, an anti-TIM3 treatment, and an anti CTLA-4 treatment.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1A is a schematic representation of an anti-EGFR 806-4-1BBz CAR lentiviral vector construct. FIG. 1B is a representative histogram showing EGFR-specific 806 BBz CAR surface expression in primary CD4⁺ and CD8⁺ T cells following transduction with CAR-encoding lentiviral vector. CAR expression was analyzed by flow cytometry using biotinylated goat-anti-mouse F(ab)2 followed by streptavidin-APC.

FIG. 2 illustrates 806 CAR activity against human GBM cells. Antigen specific cytolytic activity of 806-4-1BBz CAR T cells in EGFR, EGFRvIII-, and EGFR^{A289V}-expressing GBM cell lines was assessed in 4 hour chromium release assays using different T cell to tumor cell ratios. EGFRvIII specific 2173 and Cetuximab (C225) CAR which recognize EGFRvIII and EGFR wild type, respectively, were used as positive controls. CD19 BBz CART cells were used as negative control. U87 wtEGFR is the U87 MG parental GBM cell line that has a basal level of EGFR transduced with overexpressed wildtype EGFR.

FIG. 3 illustrates in vitro cytolysis of 806 4-1BBz CAR T cells in U87 MG cell lines transduced with EGFRvIII and EGFR missense mutation R108K in 4hr chromium release assay. Wild type EGFR specific C10 4-1BBz and EGFRVIII-specific 2173 4-1BBz CARs were used as positive controls and CD19 4-1BBz CAR was used as a negative control.

FIG. 4A is a schematic representation of an 806 KIR CAR lentiviral vector construction. FIG. 4B is a representative histogram showing CAR expression. Primary human T cells were simulated for 24 hours with anti-CD³/anti-CD28 T-cell activating beads. T cells were transduced with a lentiviral vector encoding 806 KIR CAR and cells were expanded for 10 days. CAR expression was analyzed by flow cytometry using biotinylated goat-anti-mouse F(ab)2 followed by streptavidin-APC.

FIG. 5 illustrates antigen specific cytolytic activity of 806 KIR CAR T cells in EGFR, EGFRvIII, and EGFR^{A289v} expressing GBM cell lines in an overnight luciferase based assay, detecting live tumor cells expressing luciferase. EGFRvIII-specific 2173 and wtEGFR-specific Cetuximab (C225) CARs were used as positive controls and T cells without CARs (untransduced) were used as a negative control.

FIG. 6A illustrates surface staining of wild type EGFR in parental U87 MG GBM cells and EGFR transduced clones. FIG. 6B illustrates surface staining of EGFRvIII variant with an EGFRvIII-specific antibody.

FIG. 7 is a schematic illustrating missense mutations in the extracellular domain of EGFR observed in human GBM tumors. For the EGFRvIII variant, exons 2-7 are deleted from full length EGFR.

FIG. 8 is a schematic of a lentiviral vector that co-expresses CFP and EGFR mutants.

FIG. 9 illustrates co-expression of EGFR missense mutants in U87 MG cell line transduced with wtEGFR (U87 wtEGFR). Indicated EGFR missense mutations specific to the extracellular domain were introduced into the EGFR gene by Geneart gene synthesis and site directed mutagenesis (Thermo fisher). Lentiviral vectors co-expressing

CFP and EGFR mutations (FIG. 8) were transduced into U87 wtEGFR and CFP positive cells were sorted by flourescence activated cell sorting.

FIG. 10 illustrates co-expression of EGFR missense mutants in U87 MG cell line. Indicated EGFR missense mutations specific to the extracellular domain were introduced into the EGFR gene by Geneart gene synthesis and site directed mutagenesis (Thermo fisher). Lentiviral vectors co-expressing CFP and EGFR mutations (FIG. 8) were transduced into U87 MG and CFP positive cells were sorted by fluorescence activated cell sorting.

FIG. 11 illustrates amino acid sequences for the heavy and light chains of Chimeric 806 (SEQ ID NOs: 34 and 33, respectively), Humanized 806 (SEQ ID NOs: 26 and 27), and Affinity Maturated Humanized 806 (SEQ ID NOs: 29 and 30).

FIG. 12 illustrates the DNA sequences for the heavy and light chains of Chimeric 806 (SEQ ID NOs: 4 and 3, respectively), Humanized 806 (SEQ ID NOs: 23 and 24), and Affinity Maturated Humanized 806 (SEQ ID NOs: 62 and 63).

FIG. 13 illustrates the DNA (SEQ ID NO: 64) and amino acid (SEQ ID NO; 65) sequences for the entire Chimeric 806 CAR construct.

FIG. 14 illustrates the DNA (SEQ ID NO: 66) and amino acid sequences (SEQ ID NO: 67) for the entire Humanized 806 CAR construct.

FIG. 15 illustrates the DNA (SEQ ID NO: 68) and amino acid (SEQ ID NO: 69) sequences for the entire Affinity Maturated Humanized 806 CAR construct.

FIGS. 16A-16B illustrate data from experiments wherein subcutaneous tumors were treated with combination therapy of 806-BBz CAR and anti-PD1 antibody. FIG. 16A shows data from subcutaneous U87 wtEGFR/EGFRvIII cell lines treated with combinations of either PBS or anti-PD-1 antibody and untransduced T cells or 806 BBz CAR T cells. Combination therapy demonstrated a larger decrease in relative tumor change, as determined by bioluminescence. FIG. 16B shows % tumor change, relative to PBS+untransduced (UTD) cells, on Day 16 post-CAR T infusion.

FIGS. 17A-17C illustrate in vivo anti-tumor activity of 806 KIR, against (FIG. 17A) U87 wtEGFR and (FIG. 17B) U87 wtEGFR/EGFRvIII flank tumors. Tumor models had overexpression of wildtype EGFR, either alone or in the presence of accompanying EGFR mutations.This pairing is a more physiologic representation than sole expression of the EGFR mutation, in the absence of overexpression of wildtype EGFR. FIG. 17C shows in vivo anti-tumor activity of 806 BBz, against U87 wtEGFR/EGFRvIII flank tumors.

FIGS. 18A-18B illustrate in vitro efficacy of 806 CART cells. FIG. 18A shows antigen specific cytolytic activity of 806 and 2173 CAR T cells in EGFR and its variants EGFRvIII, EGFR^{R108K/G} and EGFR^{A289D/T/V} expressing U87MG and U87 wtEGFR cell lines in 24 hour luciferase assay at indicated effector to target ratios. C225 BBz and C225 KIR CARS, which recognize wtEGFR, EGFRvIII, and its mutant variants, were used as positive control and CD19 BBz CAR as negative control. FIG. 18B shows antigen specific cytolytic activity of 806 and 2173 CAR T cells in EGFR and its variants expressing K562 cells in a 4 hour chromium release assay at indicated effector to target ratio. K562 cells express no basal EGFR, providing a clean background against which to test antigen specificity.

FIGS. 19A-19C illustrate in vitro efficacy of 806 CART cells. FIGS. 19A-19B show results from experiments wherein K562 cells expressing wtEGFR, EGFRvIII, or EGFR-mutants were co-cultured with 806 CAR T cells for 48 hours and IFN-γ, TNF-α and IL2 secretion was measured by ELISA. FIG. 19C shows CD107a degranulation of CAR T cells when co-cultured for 4 hours with K562 cells expressing wtEGFR, EGFRvIII, or an EGFR mutant. Results presented as percentage of CD107a expression on CD3⁺cells.

FIGS. 20A-20C illustrate anti-tumor efficacy of 806 CAR T cells in primary astrocytes and keratinocytes. FIG. 20A shows surface expression of EGFR assessed by flow-cytometry on human primary astrocytes and keratinocytes. FIG. 20B shows results from experiments wherein primary astrocytes and keratinocytes were co-cultured with 806 CAR T cells at indicated ratios in a 4 hour chromium assay. FIG. 20C shows results from experiments wherein primary astrocytes and keratinocytes were co-cultured with 806 CAR T cells at effector to target ratio of 1:5 and IFN-γ was measured from supernatants after 24 hours incubation at 37° C.

FIGS. 21A-21C illustrate patient-derived GBM organoids (GBOs) co-cultured with CAR T cells. FIG. 21A shows immunofluorescence staining of GBO at 24 and 72 hours after co-culture with 806 BBZ, 2173 BBz, and CD19 BBz CAR T cells. FIGS. 21B-21C show quantification of cell immunostaining for (FIG. 21B) CD3 and (FIG. 21C) cleaved-caspase 3. Although there was not a significant difference in CD3 expression, caspase activity was significantly different, suggesting 806 BBz CAR T cells led to increased tumor killing compared to 2173 BBz CAR T cells. 8167 GBO expressed endogenous amplified wtEGFR, EGFRvIII, and EGFR^{A289V}, portraying a more physiologic representation of GBMs than standard glioma stem cell lines.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

“Allogeneic” refers to any material derived from a different animal of the same species. “Xenogeneic” refers to any material derived from an animal of a different species.

The term “chimeric antigen receptor” or “CAR,” as used herein, refers to an artificial cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CARs has specificity to a selected target, for example a B cell surface receptor. CARs may also comprise an intracellular activation domain, a transmembrane domain and an extracellular domain comprising a tumor associated antigen binding region, In some aspects, CARs comprise an extracellular domain comprising an anti-B cell binding domain fused to CD3-zeta transmembrane and intracellular domain

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system. As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. “Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

When “an immunologically effective amount,” “an autoimmune disease-inhibiting effective amount,” or “therapeutic amount” is indicated, the precise amount of the compositions of the present invention to be administered can be determined by a physician or researcher with consideration of individual differences in age, weight, tumor size, extent of infection or metastasis, and condition of the patient (subject).

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

As used herein, the term “isoform” means any of two or more functionally similar proteins that have a similar but not identical amino acid sequence and are either encoded by different genes or by RNA transcripts from the same gene which have had different exons removed.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“KIR” means killer cell immunoglobulin-like receptor. KIRs have been characterized in humans and non-human primates, and are polymorphic type 1 trans-membrane molecules present on certain subsets of lymphocytes, including NK cells and some T cells. KIRs regulate the killing function of NK cells by interacting with determinants in the alpha 1 and 2 domains of the MEW class I molecules. This interaction allows them to detect virus infected cells or tumor cells. Most KIRs are inhibitory, meaning that their recognition of MEW suppresses the cytotoxic activity of the NK cell that expresses them. Only a limited number of KIRs have the ability to activate cells. The KIR gene family has at least 15 gene loci (KIR2DL1, KIR2DL2/L3, KIR2DL4, KIR2DL5A, KIR2DL5B, KIR2DS1, KIR2DS2, KIR2DS3, KIR2DS4, KIR2DS5, KIR3DL1/S1, KIR3DL2, KIR3DL3) and two pseudogenes (KIR2DP1 and KIR3DP1) encoded within a 100-200 Kb region of the Leukocyte Receptor Complex (LRC) located on chromosome 19 (19q13.4). The LRC constitutes a large, 1 Mb, and dense cluster of rapidly evolving immune genes which contains genes encoding other cell surface molecules with distinctive Ig-like extra-cellular domains. In addition, the extended LRC contains genes encoding the transmembrane adaptor molecules DAP10 and DAP12.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like.

Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodirner of an alpha (a) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state. The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

By “local” or “locally” as they are used herein to refer to delivery of a treatment, is meant intrathecal, intratumoral, or other forms of treatment delivery that are not parenteral.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention provides high affinity chimeric antigen receptors (CARs) with cross-reactivity to clinically-relevant EGFR mutated proteins, and methods of use thereof. In certain embodiments, the CAR comprises an antigen binding domain capable of binding multiple isoforms of epidermal growth factor receptor (EGFR), a transmembrane domain, and an intracellular signaling domain. In certain embodiments, the CAR is a humanized CAR. In certain embodiments, the CAR is an affinity maturated, humanized CAR. In certain embodiments, the CAR is a KIR CAR. In certain aspects, the invention includes methods of treating cancer in a subject in need thereof by administering a CAR of the present invention.

### Chimeric Antigen Receptor (CAR)

The present invention provides chimeric antigen receptors (CARs) capable of binding/having affinity for multiple EGFR isoforms. Also provided are nucleic acids encoding said CARs, vectors encoding said nucleic acids, and modified cells (e modified T cells) comprising said CARs, vectors, or nucleic acids.

A subject CAR of the invention comprises an antigen binding domain capable of binding multiple isoforms of EGFR, a transmembrane domain, and an intracellular domain. A subject CAR of the invention may optionally comprise a hinge domain.

Accordingly, a subject CAR of the invention comprises an antigen binding domain capable of binding multiple isoforms of EGFR, a hinge domain, a transmembrane domain, and an intracellular domain. In certain embodiments, each of the domains of a subject CAR is separated by a linker.

The antigen binding domain may be operably linked to another domain of the CAR, such as the transmembrane domain, or the intracellular domain, each described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding a costimulatory signaling domain.

The antigen binding domains described herein can be combined with any of the transmembrane domains, any of the intracellular domains, or any of the other domains described herein that may be included in a CAR of the present invention.

In one aspect, the invention includes an isolated CAR comprising an antigen binding domain capable of binding multiple isoforms of epidermal growth factor receptor

(EGFR), a transmembrane domain, and an intracellular domain. EGFR isoforms that the CAR is capable of binding to include but are not limited to wild-type EGFR (wtEGFR), mutated EGFR, EGFR^{A289V}, EGFR^{A289D}, EGFR^{A289T}, EGFR^{R108K}, EGFR^{R108G}, EGFR^{G598V}, EGFR^{D126Y}, EGFR^{C628F}, EGFR^{R108K/A289V}, EGFR^{R108k/D126Y}, EGFR^{A289V/G598V}, EGFR^{A289V/C628F}, and EGFR variant II.

In one aspect, the invention includes an isolated CAR comprising an antigen binding domain capable of binding multiple isoforms of EGFR, a CD8 hinge domain, a CD8 transmembrane domain, and a 4-1BBZ intracellular domain. In another aspect, the invention includes an isolated nucleic acid encoding a CAR, wherein the CAR comprises an antigen binding domain capable of binding multiple isoforms of EGFR, a CD8 hinge domain, a CD8 transmembrane domain, and a 4-1BBZ intracellular domain. Another aspect of the invention includes an isolated polypeptide comprising a CAR, wherein the CAR comprises an antigen binding domain capable of binding multiple isoforms of

EGFR, a CD8 hinge domain, a CD8 transmembrane domain, and a 4-1BBZ intracellular domain.

In one aspect, the invention includes a killer immunoglobulin-like receptor (KIR)-based CAR. KIR-CARS are based on the based upon the killer immunoglobulin-like receptors (KIRs) normally expressed by natural killer (NK) cells. In certain embodiments, the invention includes a KIR-CAR comprising an antigen binding domain capable of binding multiple isoforms of EGFR, a KIR transmembrane domain, and a KIR intracellular (cytoplasmic) domain. In certain embodiments, the invention includes an isolated nucleic acid encoding a KIR-CAR, wherein the KIR-CAR comprises an antigen binding domain capable of binding multiple isoforms of EGFR, a KIR transmembrane domain, and a KIR intracellular (cytoplasmic) domain. In certain embodiments, the invention includes an isolated polypeptide comprising KIR-CAR, wherein the KIR-CAR comprises an antigen binding domain capable of binding multiple isoforms of EGFR, a KIR transmembrane domain, and a KIR intracellular (cytoplasmic) domain.

In one aspect, the invention includes a humanized EGFR CAR In one aspect, the invention includes an affinity maturated, humanized EGFR CAR.

TABLE 1
Amino acid and nucleotide sequences.

| SEQ | | |
|---|---|---|
| ID | | |
| NO: | | |
| 1 | 806-scFv | GATGTCCAGCTGCAAGAGTCTGGCCCTAGCCTGGTCAAGCCTA |
| | (VH > VL) | GCCAGAGCCTGAGCCTGACATGTACCGTGACCGGCTACAGCA |
| | | TCACCAGCGACTTCGCCTGGAACTGGATCAGACAGTTCCCCGG |
| | | CAACAAGCTGGAATGGATGGGCTACATCAGCTACAGCGGCAA |
| | | CACCCGGTACAACCCCAGCCTGAAGTCCCGGATCTCCATCACC |
| | | AGAGACACCAGCAAGAACCAGTTCTTCCTGCAGCTGAACAGC |
| | | GTGACCATCGAGGACACCGCCACCTACTACTGTGTGACAGCCG |
| | | GCAGAGGCTTCCCTTATTGGGGACAGGGAACCCTGGTCACAGT |
| | | GTCTGCTGGTGGCGGAGGATCTGGCGGAGGCGGATCTTCTGGC |
| | | GGTGGCTCTGATATCCTGATGACACAGAGCCCCAGCAGCATGT |
| | | CTGTGTCCCTGGGCGATACCGTGTCCATCACCTGTCACAGCAG |
| | | CCAGGACATCAACAGCAACATCGGCTGGCTGCAGCAGAGGCC |
| | | TGGCAAGTCTTTTAAGGGCCTGATCTACCACGGCACCAACCTG |
| | | GATGATGAGGTGCCCAGCAGATTTTCCGGCTCTGGAAGCGGA |
| | | GCCGACTACTCCCTGACAATCAGCAGCCTGGAAAGCGAGGAC |
| | | TTCGCCGATTACTACTGCGTGCAGTACGCCCAGTTTCCTTGGA |
| | | CCTTTGGAGGCGGCACAAAGCTGGAAATCAAGCGG |
| 2 | 806-scFv | DVQLQESGPSLNKPSQSLSLTCTVTGYSITSDFAWNWIRQFPGNK |
| | (VH > VL) | LEWMGYISYSGNTRYNPSLKSRISITRDTSKNQFFLQLNSVTIEDT |
| | | ATYYCVTAGRGFPYWGQGTLVTVSAGGGGSGGGGSSGGGSDIL |
| | | MTQSPSSMSVSLGDTVSITCHSSQDINSNIGWLQQRPGKSFKGLIY |
| | | HGTNLDDEVPSRFSGSGSGADYSLTISSLESEDFADYYCVQYAQF |
| | | PWTFGGGTKLEIKR |
| 31 | 806 scFv | GATATTCTGATGACTCAATCTCCGTCTTCTATGAGCGTGAGCTT |
| | (VL > VH) | GGGTGACACCGTCAGCATCACCTGTCATTCCAGCCAGGATATA |
| | | AACTCAAATATCGGCTGGCTCCAGCAACGCCCAGGCAAGTCA |
| | | TTCAAGGGGCTTATTTATCATGGCACCAATCTTGACGATGAAG |
| | | TCCCATCACGCTTCAGCGGATCAGGCTCAGGTGCGGACTATTC |
| | | CTTGACTATAAGTTCCCTCGAATCTGAGGATTTCGCCGACTAT |
| | | TATTGCGTACAATACGCCCAGTTTCCCTGGACCTTCGGAGGCG |
| | | GCACCAAATTGGAGATAAAAAGGGGTGGAGGAGGATCAGGC |
| | | GGGGGTGGAAGCGGCGGAGGAGGCAGCGACGTACAACTGCA |
| | | AGAATCCGGGCCGAGTTTGGTCAAGCCCTCTCAATCTCTTTCT |
| | | CTCACTTGCACGGTCACCGGATACTCCATAACCAGCGATTTTG |
| | | CGTGGAATTGGATTCGACAATTTCCAGGGAATAAATTGGAATG |
| | | GATGGGATATATCAGTTATTCTGGTAATACCAGATACAACCCG |
| | | TCATTGAAAAGTCGCATCTCTATAACACGAGACACTTCAAAGA |
| | | ATCAGTTCTTCCTTCAGCTCAATTCTGTAACCATCGAAGATACT |
| | | GCTACTTATTACTGTGTAACGGCGGGTCGAGGATTCCCCTACT |
| | | GGGGCCAGGGTACACTGGTTACTGTTTCCGCC |
| 32 | 806 scFv | DILMTQSPSSMSVSLGDTVSITCHSSQPINSNIGWLQQRPGKSFKG |
| | (VL > VH) | LIYHGTNLDDEVPSRFSGSGSGADYSLTISSLESEDFADYYCVQY |
| | | AQFPWTFGGGTKLEIKRGGGGSGGGGSGGGGSDVQLQESGPSLV |
| | | KPSQSLSLTCTVTGYSITSDFAWNWIRQFPGNKLEWMGYISYSGN |
| | | TRYNPSLKSRISITRDTSKNQFFLQLNSVTIEDTATYYCVTAGRGF |
| | | PYWGQGTLVTVSA |
| 3 | Chimeric 806 | DILMTQSPSSMSVSLGDTVSITCHSSQDINSNIGWLQQRPGKSFKG |
| | VL | LIYHGTNLDDEVPSRFSGSGSGADYSLTISSLESEDFADYYCVQY |
| | | AQFPWTFGGGTKLEIKR |
| 33 | Chimeric 806 | gatatcctgatgacacagagccccagcagcatgtctgtgtccctgggcgataccgtgtccatcacctgtc |
| | VL | acagcagccaggacatcaacagcaacatcggctggctgcagcagaggcctggcaagtcttttaaggg |
| | | cctgatctaccacggcaccaacctggatgatgaggtgcccagcagattttccggctctggaagcggag |
| | | ccgactactccctgacaatcagcagcctggaaagcgaggacttcgccgattactactgcgtgcagtacg |
| | | cccagtttccttggacctttggaggcggcacaaagctggaaatcaagcgg |
| 4 | Chimeric 806 | DVQLQESGPSLVKPSQSLSLTCTVTGYSITSDFAWNWIRQFPGNK |
| | VH | LEWMGYISYSGNTRYNPSLKSRISITRDTSKNQFFLQLNSVTIEDT |
| | | ATYYCVTAGRGFPYWGQGTLVTVSA |
| 34 | Chimeric 806 | gatgtccagctgcaagagtctggccctagcctggtcaagcctagccagagcctgagcctgacatgtac |
| | VH | cgtgaccggctacagcatcaccagcgacttcgcctggaactggatcagacagttccccggcaacaagc |
| | | tggaatggatgggctacatcagctacagcggcaacacccggtacaaccccagcctgaagtcccggat |
| | | ctccatcaccagagacaccagcaagaaccagttcttcctgcagctgaacagcgtgaccatcgaggaca |
| | | ccgccacctactactgtgtgacagccggcagaggcttcccttattggggacagggaaccctggtcaca |
| | | gtgtctgct |
| 5 | LCDR1 | HSSQDINSNIG |
| 6 | LCDR2 | HGTNLDD |
| 7 | LCDR3 | VQYAQFPWT |
| 8 | HCDR1 | GYSITSDFAWN |
| 9 | HCDR2 | GYISYSGNTRYNPSLK |
| 10 | HCDR3 | VTAGRGFPYW |
| 11 | CD8 hinge | ACCACTACCCCAGCACCGAGGCCACCCACCCCGGCTCCTACCA |
| | | TCGCCTCCCAGCCTCTGTCCCTGCGTCCGGAGGCATGTAGACC |
| | | CGCAGCTGGTGGGGCCGTGCATACCCGGGGTCTTGACTTCGCC |
| | | TGCGAT |
| 71 | CD8 hinge | ACCACGACGCCAGCGCCGCGACCACCAACACCGGCGCCCACC |
| | | ATCGCGTCGCAGCCCCTGTCCCTGCGCCCAGAGGCGTGCCGGC |
| | | CAGCGGCGGGGGGCGCAGTGCACACGAGGGGGCTGGACTTCG |
| | | CCTGTGAT |
| 72 | CD8 hinge | TTTPAPRPPTPAPTIASQPLSLRPEACRPAAGGAVHTRGLDFACD |
| 12 | CD8 | ATCTACATTTGGGCCCCTCTGGCTGGTACTTGCGGGGTCCTGC |
| | transmembrane | TGCTTTCACTCGTGATCACTCTTTACTGT |
| | domain | |
| 73 | CD8 | ATCTACATCTGGGCCCCTCTGGCCGGCACCTGTGGCGTGCTGC |
| | transmembrane | TGCTGTCCCTGGTCATCACCCTGTACTGC |
| | domain | |
| 74 | CD8 | IYIWAPLAGTCGVLLLSLVITLYC |
| | transmembrane | |
| | domain | |
| 13 | 4-1BB | AAGCGCGGTCGGAAGAAGCTGCTGTACATCTTTAAGCAACCCT |
| | intracellular | TCATGAGGCCTGTGCAGACTACTCAAGAGGAGGACGGCTGTT |
| | domain | CATGCCGGTTCCCAGAGGAGGAGGAAGGCGGCTGCGAACTG |
| 75 | 4-1BB | AAGCGGGGCAGAAAGAAGCTGCTGTACATCTTCAAGCAGCCC |
| | intracellular | TTCATGCGGCCTGTGCAGACCACACAGGAAGAGGACGGCTGT |
| | domain | AGCTGTAGATTCCCCGAGGAAGAGGAAGGCGGCTGCGAGCTG |
| 76 | 4-1BB | KRGRKKLLYIFKQPFMRPVQTTQEEDGCSCRFPEEEEGGCEL |
| | intracellular | |
| | domain | |
| 14 | CD3-zeta | CGCGTGAAATTCAGCCGCAGCGCAGATGCTCCAGCCTACAAG |
| | | CAGGGGCAGAACCAGCTCTACAACGAACTCAATCTTGGTCGG |
| | | AGAGAGGAGTACGACGTGCTGGACAAGCGGAGAGGACGGGA |
| | | CCCAGAAATGGGCGGGAAGCCGCGCAGAAAGAATCCCCAAGA |
| | | GGGCCTGTACAACGAGCTCCAAAAGGATAAGATGGCAGAAGC |
| | | CTATAGCGAGATTGGTATGAAAGGGGAACGCAGAAGAGGCAA |
| | | AGGCCACGACGGACTGTACCAGGGACTCAGCACCGCCACCAA |
| | | GGACACCTATGACGCTCTTCACATGCAGGCCCTGCCGCCTCGG |
| 77 | CD3-zeta | AGAGTGAAGTTCAGCAGAAGCGCCGACGCCCCTGCCTATCAG |
| | | CAGGGCCAGAACCAGCTGTACAACGAGCTGAACCTGGGCAGA |
| | | CGGGAGGAATACGACGTGCTGGACAAGAGAAGAGGCCGGGA |
| | | CCCTGAGATGGGCGGCAAGCCCAGACGGAAGAACCCCCAGGA |
| | | AGGCCTGTATAACGAACTGCAGAAAGACAAGATGGCCGAGGC |
| | | CTACAGCGAGATCGGCATGAAGGGCGAGCGGAGAAGAGGCA |
| | | AGGGCCATGACGGCCTGTACCAGGGCCTGAGCACCGCCACCA |
| | | AGGACACCTACGACGCCCTGCACATGCAGGCCCTGCCTCCAA |
| | | GA |
| 78 | CD3-zeta | RVKFSRSADAPAYQQGQNQLYNELNLGRREEYDVLDKRRGRDP |
| | | EMGGKPRRKNPQEGLYNELQKDKMAEAYSEIGMKGERRRGKG |
| | | HDGLYQGLSTATKDTYDALHMQALPPR |
| 15 | CD8 signal | ATGGCCTTACCAGTGACCGCCTTGCTCCTGCCGCTGGCCTTGC |
| | recognition | TGCTCCACGCCGCCAGGCCG |
| | peptide | |
| 70 | CD8 signal | MALPVTALLLPLALLLHAARP |
| | recognition | |
| | peptide | |
| 16 | DAP12 | ATGGGGGGACTTGAACCCTGCAGCAGGTTCCTGCTCCTGCCTC |
| | | TCCTGCTGGCTGTAAGTGGTCTCCGTCCTGTCCAGGTCCAGGC |
| | | CCAGAGCGATTGCAGTTGCTCTACGGTGAGCCCGGGCGTGCTG |
| | | GCAGGGATCGTGATGGGAGACCTGGTGCTGACAGTGCTCATT |
| | | GCCCTGGCCGTGTACTTCCTGGGCCGGCTGGTCCCTCGGGGGC |
| | | GAGGGGCTGCGGAGGCAGCGACCCGGAAACAGCGTATCACTG |
| | | AGACCGAGTCGCCTTATCAGGAGCTCCAGGGTCAGAGGTCGG |
| | | ATGTCTACAGCGACCTCAACACACAGAGGCCGTATTACAAA |
| 17 | T2A | GTCGAGGGCGGCGGAGAGGGCAGAGGAAGTCTTCTAACATGC |
| | | GGTGACGTGGAGGAGAATCCCGGCCCTAGG |
| 18 | Linker + | GGTGGCGGAGGTTCTGGAGGTGGGGGTTCCTCACCCACTGAA |
| | KIRS2 | CCAAGCTCCAAAACCGGTAACCCCAGACACCTGCATGTTCTGA |
| | | TTGGGACCTCAGTGGTCAAAATCCCTTTCACCATCCTCCTCTTC |
| | | TTTCTCCTTCATCGCTGGTGCTCCAACAAAAAAAATGCTGCTG |
| | | TAATGGACCAAGAGCCTGCAGGGAACAGAACAGTGAACAGCG |
| | | AGGATTCTGATGAACAAGACCATCAGGAGGTGTCATACGCAT |
| | | AA |
| 19 | 806-BBZ- | ATGGCCTTACCAGTGACCGCCTTGCTCCTGCCGCTGGCCTTGC |
| | CAR | TGCTCCACGCCGCCAGGCCGGGATCCGATGTCCAGCTGCAAG |
| | | AGTCTGGCCCTAGCCTGGTCAAGCCTAGCCAGAGCCTGAGCCT |
| | | GACATGTACCGTGACCGGCTACAGCATCACCAGCGACTTCGCC |
| | | TGGAACTGGATCAGACAGTTCCCCGGCAACAAGCTGGAATGG |
| | | ATGGGCTACATCAGCTACAGCGGCAACACCCGGTACAACCCC |
| | | AGCCTGAAGTCCCGGATCTCCATCACCAGAGACACCAGCAAG |
| | | AACCAGTTCTTCCTGCAGCTGAACAGCGTGACCATCGAGGACA |
| | | CCGCCACCTACTACTGTGTGACAGCCGGCAGAGGCTTCCCTTA |
| | | TTGGGGACAGGGAACCCTGGTCACAGTGTCTGCTGGTGGCGG |
| | | AGGATCTGGCGGAGGCGGATCTTCTGGCGGTGGCTCTGATATC |
| | | CTGATGACACAGAGCCCCAGCAGCATGTCTGTGTCCCTGGGCG |
| | | ATACCGTGTCCATCACCTGTCACAGCAGCCAGGACATCAACAG |
| | | CAACATCGGCTGGCTGCAGCAGAGGCCTGGCAAGTCTTTTAAG |
| | | GGCCTGATCTACCACGGCACCAACCTGGATGATGAGGTGCCC |
| | | AGCAGATTTTCCGGCTCTGGAAGCGGAGCCGACTACTCCCTGA |
| | | CAATCAGCAGCCTGGAAAGCGAGGACTTCGCCGATTACTACT |
| | | GCGTGCAGTACGCCCAGTTTCGTTGGACcrTTGGAGGCGGCAC |
| | | AAAGCTGGAAATCAAGCGGGCTAGCACCACTACCCCAGCACC |
| | | GAGGCCACCCACCCCGGCTCCTACCATCGCCTCCCAGCCTCTG |
| | | TCCCTGCGTCCGGAGGCATGTAGACCCGCAGCTGGTGGGGCC |
| | | GTGCATACCCGGGGTCTTGACTTCGCCTGCGATATCTACATTT |
| | | GGGCCCCTCTGGCTGGTACTTGCGGGGTCCTGCTGCTTTCACT |
| | | CGTGATCACTCTTTACTGTAAGCGCGGTCGGAAGAAGCTGCTG |
| | | TACATCTTTAAGCAACCCTTCATGAGGCCTGTGCAGACTACTC |
| | | AAGAGGAGGACGGCTGTTCATGCCGGTTCCCAGAGGAGGAGG |
| | | AAGGCGGCTGCGAACTGCGCGTGAAATTCAGCCGCAGCGCAG |
| | | ATGCTCCAGCCTACAAGCAGGGGCAGAACCAGCTCTACAACG |
| | | AACTCAATCTTGGTCGGAGAGAGGAGTACGACGTGCTGGACA |
| | | AGCGGAGAGGACGGGACCCAGAAATGGGCGGGAAGCCGCGC |
| | | AGAAAGAATCCCCAAGAGGGCCTGTACAACGAGCTCCAAAAG |
| | | GATAAGATGGCAGAAGCCTATAGCGAGATTGGTATGAAAGGG |
| | | GAACGCAGAAGAGGCAAAGGCCACGACGGACTGTACCAGGG |
| | | ACTCAGCACCGCCACCAAGGACACCTATGACGCTCTTCACATG |
| | | CAGGCCCTGCCGCCTCGGTGA |
| 20 | 806-BBZ- | MALPVTALLLPLALLLHAARPGSDVQLQESGPSLVKPSQSLSLTC |
| | CAR | TVTGYSITSDFAWNWIRQFPGNKLEWMGYISYSGNTRYNPSLKS |
| | | RISITRDTSKNQFFLQLNSVTIEDTATYYCVTAGRGFPYWGQGTL |
| | | VTVSAGGGGSGGGGSSGGGSDILMTQSPSSMSVSLGDTVSITCHS |
| | | SQDINSNIGWLQQRPGKSFKGLIYHGTNLDDEVPSRFSGSGSGAD |
| | | YSLTISSLESEDFADYYCVQYAQFPWTFGGGTKLEIKRASTTTPAP |
| | | RPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGC |
| | | SCRFPEEEEGGCELRVKFSRSADAPAYKQGQNQLYNELNLGRRE |
| | | EYDVLDKRRGRDPEMGGKPRRKNPQEGLYNELQKDKMAEAYSE |
| | | IGMKGERRRGKGHDGLYQGLSTATKDTYDALHMQALPPR |
| 21 | 806-KIR- | ATGGGGGGACTTGAACCCTGCAGCAGGTTCCTGCTCCTGCCTC |
| | CAR | TCCTGCTGGCTGTAAGTGGTCTCCGTCCTGTCCAGGTCCAGGC |
| | | CCAGAGCGATTGCAGTTGCTCTACGGTGAGCCCGGGCGTGCTG |
| | | GCAGGGATCGTGATGGGAGACCTGGTGCTGACAGTGCTCATT |
| | | GCCCTGGCCGTGTACTTCCTGGGCCGGCTGGTCCCTCGGGGGC |
| | | GAGGGGCTGCGGAGGCAGCGACCCGGAAACAGCGTATCACTG |
| | | AGACCGAGTCGCCTTATCAGGAGCTCCAGGGTCAGAGGTCGG |
| | | ATGTCTACAGCGACCTCAACACACAGAGGCCGTATFACAAAG |
| | | TCGAGGGCGGCGGAGAGGGCAGAGGAAGTCTTCTAACATGCG |
| | | GTGACGTGGAGGAGAATCCCGGCCCTAGGATGGCCTTACCAG |
| | | TGACCGCCTTGCTCCTGCCGCTGGCCTTGCTGCTCCACGCCGC |
| | | CAGGCCGGGATCCGATGTCCAGCTGCAAGAGTCTGGCCCTAG |
| | | CCTGGTCAAGCCTAGCCAGAGCCTGAGCCTGACATGTACCGTG |
| | | ACCGGCTACAGCATCACCAGCGACTTCGCCTGGAACTGGATCA |
| | | GACAGTTCCCCGGCAACAAGCTGGAATGGATGGGCTACATCA |
| | | GCTACAGCGGCAACACCCGGTACAACCCCAGCCTGAAGTCCC |
| | | GGATCTCCATCACCAGAGACACCAGCAAGAACCAGTTCTTCCT |
| | | GCAGCTGAACAGCGTGACCATCGAGGACACCGCCACCTACTA |
| | | CTGTGTGACAGCCGGCAGAGGCTTCCCTTATTGGGGACAGGG |
| | | AACCCTGGTCACAGTGTCTGCTGGTGGCGGAGGATCTGGCGG |
| | | AGGCGGATCTTCTGGCGGTGGCTCTGATATCCTGATGACACAG |
| | | AGCCCCAGCAGCATGTCTGTGTCCCTGGGCGATACCGTGTCCA |
| | | TCACCTGTCACAGCAGCCAGGACATCAACAGCAACATCGGCT |
| | | GGCTGCAGCAGAGGCCTGGCAAGTCTTTTAAGGGCCTGATCTA |
| | | CCACGGCACCAACCTGGATGATGAGGTGCCCAGCAGATTTTCC |
| | | GGCTCTGGAAGCGGAGCCGACTACTCCCTGACAATCAGCAGC |
| | | CTGGAAAGCGAGGACTTCGCCGATTACTACTGCGTGCAGTACG |
| | | CCCAGTTTCCTTGGACCTTTGGAGGCGGCACAAAGCTGGAAAT |
| | | CAAGCGGGCTAGCGGTGGCGGAGGTTCTGGAGGTGGGGGTTC |
| | | CTCACCCACTGAACCAAGCTCCAAAACCGGTAACCCCAGACA |
| | | CCTGCATGTTCTGATTGGGACCTCAGTGGTCAAAATCCCTTTC |
| | | ACCATCCTCCTCTTCTTTCTCCTTCATCGCTGGTGCTCCAACAA |
| | | AAAAAATGCTGCTGTAATGGACCAAGAGCCTGCAGGGAACAG |
| | | AACAGTGAACAGCGAGGATTCTGATGAACAAGACCATCAGGA |
| | | GGTGTCATACGCATAA |
| 22 | 806-KIR- | MGGLEPCSRFLLLPLLLAVSGLRPVQVQAQSDCSCSTVSPGVLAG |
| | CAR | IVMGDLVLTVLIALAVYFLGRLVPRGRGAAEAATRKQRITETESP |
| | | YQELQGQRSDVYSDLNTQRPYYKVEGGGEGRGSLLTCGDVEEN |
| | | PGPRMALPVTALLLPLALLLHAARPGSDVQLQESGPSLVKPSQSL |
| | | SLTCTVTGYSITSDFAWNWIRQFPGNKLEWMGYISYSGNTRYNPS |
| | | LKSRISITRDTSKNQFFLQLNSVTIEDTATYYCVTAGRGFPYWGQ |
| | | GTLVTVSAGGGGSGGGGSSGGGSDILMTQSPSSMSVSLGDTVSIT |
| | | CHSSQDINSNIGWLQQRPGKSFKGLIYHGTNLDDEVPSRFSGSGS |
| | | GADYSLTISSLESEDFADYYCVQYAQFPWTFGGGTKLEIKRASGG |
| | | GGSGGGGSSPTEPSSKTGNPRHLHVLIGTSVVKIPFTILLFFLLHR |
| | | WCSNKKNAAVMDQEPAGNRTVNSEDSDEQDHQEVSYA |
| 23 | ABT-806 | CAGGTTCAGCTGCAAGAGTCTGGCCCTGGCCTGGTCAAGCCTA |
| | (humanized | GCCAAACACTGAGCCTGACCTGTACCGTGTCCGGCTACAGCAT |
| | 806) VH | CAGCAGCGACTTCGCCTGGAACTGGATCAGACAGCCTCCTGGC |
| | | AAAGGACTGGAATGGATGGGCTACATCAGCTACAGCGGCAAC |
| | | ACCAGATACCAGCCTAGCCTGAAGTCCCGGATCACCATCAGC |
| | | AGAGACACCAGCAAGAACCAGTTCTTCCTGAAGCTGAACAGC |
| | | GTGACAGCCGCCGATACCGCCACCTACTATTGTGTGACAGCTG |
| | | GCAGAGGCTTCCCCTATTGGGGACAGGGAACACTGGTCACCG |
| | | TTAGCTCT |
| 24 | ABT-806 | GATATCCAGATGACACAGAGCCCCAGCAGCATGTCCGTGTCC |
| | (humanized | GTGGGAGACAGAGTGACCATCACCTGTCACAGCAGCCAGGAC |
| | 806) VL | ATCAACAGCAACATCGGCTGGCTGCAGCAGAAGCCCGGCAAG |
| | | TCTTTTAAGGGCCTGATCTACCACGGCACCAACCTGGATGATG |
| | | GCGTGCCCAGCAGATTTTCTGGCAGCGGCTCTGGCACCGACTA |
| | | CACCCTGACCATATCTAGCCTGCAGCCTGAGGACTTCGCCACC |
| | | TATTACTGCGTGCAGTACGCCCAGTTTCCTTGGACCTTTGGAG |
| | | GCGGCACAAAGCTGGAAATCAAGCGG |
| 25 | ABT-806 | CAGGTTCAGCTGCAAGAGTCTGGCCCTGGCCTGGTCAAGCCTA |
| | (humanized | GCCAAACACTGAGCCTGACCTGTACCGTGTCCGGCTACAGCAT |
| | 806) scFv | CAGCAGCGACTTCGCCTGGAACTGGATCAGACAGCCTCCTGGC |
| | | AAAGGACTGGAATGGATGGGCTACATCAGCTACAGCGGCAAC |
| | | ACCAGATACCAGCCTAGCCTGAAGTCCCGGATCACCATCAGC |
| | | AGAGACACCAGCAAGAACCAGTTCTTCCTGAAGCTGAACAGC |
| | | GTGACAGCCGCCGATACCGCCACCTACTATTGTGTGACAGCTG |
| | | GCAGAGGCTTCCCCTATTGGGGACAGGGAACACTGGTCACCG |
| | | TTAGCTCTGATATCCAGATGACACAGAGCCCCAGCAGCATGTC |
| | | CGTGTCCGTGGGAGACAGAGTGACCATCACCTGTCACAGCAG |
| | | CCAGGACATCAACAGCAACATCGGCTGGCTGCAGCAGAAGCC |
| | | CGGCAAGTCTTTTAAGGGCCTGATCTACCACGGCACCAACCTG |
| | | GATGATGGCGTGCCCAGCAGATTTTCTGGCAGCGGCTCTGGCA |
| | | CCGACTACACCCTGACCATATCTAGCCTGCAGCCTGAGGACTT |
| | | CGCCACCTATTACTGCGTGCAGTACGCCCAGTTTCCTTGGACC |
| | | TTTGGAGGCGGCACAAAGCTGGAAATCAAGCGG |
| 79 | ABT-806 | CAGGTTCAGCTGCAAGAGTCTGGCCCTGGCCTGGTCAAGCCTA |
| | (humanized | GCCAAACACTGAGCCTGACCTGTACCGTGTCCGGCTACAGCAT |
| | 806) scFv | CAGCAGCGACTTCGCCTGGAACTGGATCAGACAGCCTCCTGGC |
| | VH > VL | AAAGGACTGGAATGGATGGGCTACATCAGCTACAGCGGCAAC |
| | | ACCAGATACCAGCCTAGCCTGAAGTCCCGGATCACCATCAGC |
| | | AGAGACACCAGCAAGAACCAGTTCTTCCTGAAGCTGAACAGC |
| | | GTGACAGCCGCCGATACCGCCACCTACTATTGTGTGACAGCTG |
| | | GCAGAGGCTTCCCCTATTGGGGACAGGGAACACTGGTCACCG |
| | | TTAGCTCTGGTGGCGGAGGATCTGGCGGAGGCGGATCTTCTGG |
| | | CGGTGGCTCTGATATCCAGATGACACAGAGCCCCAGCAGCAT |
| | | GTCCGTGTCCGTGGGAGACAGAGTGACCATCACCTGTCACAGC |
| | | AGCCAGGACATCAACAGCAACATCGGCTGGCTGCAGCAGAAG |
| | | CCCGGCAAGTCTTTTAAGGGCCTGATCTACCACGGCACCAACC |
| | | TGGATGATGGCGTGCCCAGCAGATTTTCTGGCAGCGGCTCTGG |
| | | CACCGACTACACCCTGACCATATCTAGCCTGCAGCCTGAGGAC |
| | | TTCGCCACCTATTACTGCGTGCAGTACGCCCAGTTTCCTTGGA |
| | | CCTTTGGAGGCGGCACAAAGCTGGAAATCAAGCGG |
| 81 | ABT-806 | GGCGAACTAAAGGTCGAAACACGGCGGAGGTTTCCAGGTTCC |
| | (humanized | TTTGACCCGCATGACGTGCGTCATTATCCACCGCTTCAGGAGT |
| | 806) scFv | CCGACGTCCGATCTATACCAGTCCCACATCAGCCACGGTCTCG |
| | VL > VH | GCGACGGTCTTTTAGACGACCCGTGCGGTAGTAGGTCCAACCA |
| | | CGGCACCATCTAGTCCGGGAATTTTCTGAACGGCCCGAAGACG |
| | | ACGTCGGTCGGCTACAACGACAACTACAGGACCGACGACACT |
| | | GTCCACTACCAGTGAGACAGAGGGTGCCTGTGCCTGTACGAC |
| | | GACCCCGAGACACAGTAGACCTATAGTCTCGGTGGCGGTCTTC |
| | | TAGGCGGAGGCGGTCTAGGAGGCGGTGGTCTCGATTGCCACT |
| | | GGTCACAAGGGACAGGGGTTATCCCCTTCGGAGACGGTCGAC |
| | | AGTGTGTTATCATCCACCGCCATAGCCGCCGACAGTGCGACAA |
| | | GTCGAAGTCCTTCTTGACCAAGAACGACCACAGAGACGACTA |
| | | CCACTAGGCCCTGAAGTCCGATCCGACCATAGACCACAACGG |
| | | CGACATCGACTACATCGGGTAGGTAAGGTCAGGAAACGGTCC |
| | | TCCGACAGACTAGGTCAAGGTCCGCTTCAGCGACGACTACGA |
| | | CATCGGCCTGTGCCATGTCCAGTCCGAGTCACAAACCGATCCG |
| | | AACTGGTCCGGTCCCGGTCTGAGAACGTCGACTTGGAC |
| 26 | ABT-806 | QVQ LQE SGP GLV KPS QTL SLT CTV SGY SIS SDF AWN WIR |
| | (humanized | QPP GKG LEW MGY ISY SGN TRY QPS LKS RIT ISR DTS KNQ |
| | 806) VH | FFL KLN SVT AAD TAT YYC VTA GRG FPY WGQ GTL VTV SS |
| | amino acid | |
| | sequence | |
| 27 | ABT-806 | DIQ MTQ SPSS MSVS VGDR VTIT CHSS QDIN SNIG WLQQ |
| | (humanized | KPGK SFKGLIYHG TNLD DGVP SRFS GSGS GTDY TLTI SSLQ |
| | 806) VL | PEDF ATYY CVQY AQFP WTFG GGTK LEIKR |
| 28 | ABT-806 | QVQ LQE SGP GLV KPS QTL SLT CTV SGY SIS SDF AWN WIR |
| | (humanized | QPP GKG LEW MGY ISY SGN TRY QPS LKS RIT ISR DTS KNQ |
| | 806) scFv | FFL KLN SVT AAD TAT YYC VTA GRG FPY WGQ GTL VTV |
| | VH > VL | SSDIQ MTQ SPSS MSVS VGDR VTIT CHSS QDIN SNIG WLQQ |
| | | KPGK SFKGLIYHG TNLD DGVP SRFS GSGS GTDY TLTI SSLQ |
| | | PEDF ATYY CVQY AQFP WTFG GGTK LEIKR |
| 80 | ABT-806 | QVQLQESGPGLVKPSQTLSLTCTVSGYSISSDFAWNWIRQPPGKG |
| | (humanized | LEWMGYISYSGNTRYQPSLKSRITISRDTSKNQFFLKLNSVTAAD |
| | 806) scFv | TATYYCVTAGRGFPYWGQGTLVTVSSGGGGSGGGGSSGGGSDI |
| | VH > VL | QMTQSPSSMSVSVGDRVTITCHSSQDINSNIGWLQQKPGKSFKGL |
| | | IYHGTNLDDGVPSRFSGSGSGTDYTLTISSLQPEDFATYYCVQYA |
| | | QFPWTFGGGTKLEIKR |
| 82 | ABT-806 | RKIELKTGGGFTWPFQAYQVCYYTAFDEPQLSSITLTYDTGSGSG |
| | (humanized | SFRSPVGDDLNTGHYILGKFSKGPKQQLWGINSNIDQSSHCTITVR |
| | 806) scFv | DGVSVSMSSPSQTMQIDSGGGSSGGGGSGGGGSSVTVLTGQGW |
| | VL > VH | YPFGRGATVCYYTATDAATVSNLKLFFQNKSTDRSITIRSKLSPQ |
| | | YRTNGSYSIYGMWELGKGPPQRIWNWAFDSSISYGSVTCTLSLTQ |
| | | SPKVLGPGSEQLQVQ |
| 29 | Affinity | EVQLQESGPGLVKPSQTLSLTCTVSGYSISRDFAWNWIRQPPGKG |
| | maturated | LEWMGYISYNGNTRYQPSLKSRITISRDTSKNQFFLKLNSVTAAD |
| | humanized | TATYYCVTASRGFPYWGQGTLVTVSS |
| | 806 VH | |
| 30 | Affinity | DIQMTQSPSSMSVSVGDRVTITCHSSQDINSNIGWLQQKPGKSFK |
| | maturated | GLIYHGTNLDDGVPSRFSGSGSGTDYTLTISSLQPEDFATYYCVQ |
| | humanized | YAQFPWTTFGGGTKLEIK |
| | 806 VL | |
| 62 | Affinity | GAGGTTCAGCTGCAAGAGTCTGGCCCTGGCCTGGTCAAGCCTA |
| | maturated | GCCAAACACTGAGCCTGACCTGTACCGTGTCCGGCTACAGCAT |
| | humanized | CAGCAGAGACTTCGCCTGGAACTGGATCAGACAGCCTCCTGG |
| | 806 VH | CAAAGGACTGGAATGGATGGGCTACATCAGCTACAACGGCAA |
| | | CACCAGATACCAGCCTAGCCTGAAGTCCCGGATCACCATCTCC |
| | | AGAGACACCAGCAAGAACCAGTTCTTCCTGAAGCTGAACAGC |
| | | GTGACAGCCGCCGATACCGCCACCTACTATTGTGTGACAGCCA |
| | | GCAGAGGCTTCCCCTATTGGGGACAGGGAACCCFGGTCACAG |
| | | TTAGCTCT |
| 63 | Affinity | GATATCCAGATGACACAGAGCCCCAGCAGCATGTCCGTGTCC |
| | maturated | GTGGGAGACAGAGTGACCATCACCTGTCACAGCAGCCAGGAC |
| | humanized | ATCAACAGCAACATCGGCTGGCTGCAGCAGAAGCCCGGCAAG |
| | 806 VL | TCTTTTAAGGGCCTGATCTACCACGGCACCAACCTGGATGATG |
| | | GCGTGCCCAGCAGATTTTCTGGCAGCGGCTCTGGCACCGACTA |
| | | CACCCTGACCATATCTAGCCTGCAGCCTGAGGACTTCGCCACC |
| | | TATTACTGCGTGCAGTACGCCCAGTTTCCTTGGACCTTTGGAG |
| | | GCGGCACAAAGCTGGAAATCAAG |
| 83 | Affinity | GAGGTTCAGCTGCAAGAGTCTGGCCCTGGCCTGGTCAAGCCTA |
| | maturated | GCCAAACACTGAGCCTGACCTGTACCGTGTCCGGCTACAGCAT |
| | humanized | CAGCAGAGACTTCGCCTGGAACTGGATCAGACAGCCTCCTGG |
| | 806 scFv | CAAAGGACTGGAATGGATGGGCTACATCAGCTACAACGGCAA |
| | VH > VL | CACCAGATACCAGCCTAGCCTGAAGTCCCGGATCACCATCTCC |
| | | AGAGACACCAGCAAGAACCAGTTCTTCCTGAAGCTGAACAGC |
| | | GTGACAGCCGCCGATACCGCCACCTACTATTGTGTGACAGCCA |
| | | GCAGAGGCTTCCCCTATTGGGGACAGGGAACCCTGGTCACAG |
| | | TTAGCTCTGGTGGCGGAGGATCTGGCGGAGGCGGATCTTCTGG |
| | | CGGTGGCTCTGATATCCAGATGACACAGAGCCCCAGCAGCAT |
| | | GTCCGTGTCCGTGGGAGACAGAGTGACCATCACCTGTCACAGC |
| | | AGCCAGGACATCAACAGCAACATCGGCTGGCTGCAGCAGAAG |
| | | CCCGGCAAGTCTTTTAAGGGCCTGATCTACCACGGCACCAACC |
| | | TGGATGATGGCGTGCCCAGCAGATTTTCTGGCAGCGGCTCTGG |
| | | CACCGACTACACCCTGACCATATCTAGCCTGCAGCCTGAGGAC |
| | | TTCGCCACCTATTACTGCGTGCAGTACGCCCAGTTTCCTTGGA |
| | | CCTTTGGAGGCGGCACAAAGCTGGAAATCAAG |
| 84 | Affinity | EVQLQESGPGLVKPSQTLSLTCTVSGYSISRDFAWNWIRQPPGKG |
| | maturated | LEWMGYISYNGNTRYQPSLKSRITISRDTSKNQFFLKLNSVTAAD |
| | humanized | TATYYCVTASRGFPYWGQGTLVTVSSGGGGSGGGGSSGGGSDIQ |
| | 806 scFv | MTQSPSSMSVSVGDRVTITCHSSQDINSNIGWLQQKPGKSFKGLI |
| | VH > VL | YHGTNLDDGVPSRFSGSGSGTDYTLTISSLQPEDFATYYCVQYAQ |
| | | FPWTFGGGTKLEIK |
| 85 | Affinity | GAACTAAAGGTCGAAACACGGCGGAGGTTTCCAGGTTCCTTTG |
| | maturated | ACCCGCATGACGTGCGTCATTATCCACCGCTTCAGGAGTCCGA |
| | humanized | CGTCCGATCTATACCAGTCCCACATCAGCCACGGTCTCGGCGA |
| | 806 scFv | CGGTCTTTTAGACGACCCGTGCGGTAGTAGGTCCAACCACGGC |
| | VL > VH | ACCATCTAGTCCGGGAATTTTCTGAACGGCCCGAAGACGACGT |
| | | CGGTCGGCTACAACGACAACTACAGGACCGACGACACTGTCC |
| | | ACTACCAGTGAGACAGAGGGTGCCTGTGCCTGTACGACGACC |
| | | CCGAGACACAGTAGACCTATAGTCTCGGTGGCGGTCTTCTAGG |
| | | CGGAGGCGGTCTAGGAGGCGGTGGTCTCGATTGACACTGGTC |
| | | CCAAGGGACAGGGGTTATCCCCTTCGGAGACGACCGACAGTG |
| | | TGTTATCATCCACCGCCATAGCCGCCGACAGTGCGACAAGTCG |
| | | AAGTCCTTCTTGACCAAGAACGACCACAGAGACCTCTACCACT |
| | | AGGCCCTGAAGTCCGATCCGACCATAGACCACAACGGCAACA |
| | | TCGACTACATCGGGTAGGTAAGGTCAGGAAACGGTCCTCCGA |
| | | CAGACTAGGTCAAGGTCCGCTTCAGAGACGACTACGACATCG |
| | | GCCTGTGCCATGTCCAGTCCGAGTCACAAACCGATCCGAACTG |
| | | GTCCGGTCCCGGTCTGAGAACGTCGACTTGGAG |
| 86 | Affinity | KIELKTGGGFTWPFQAYQVCYYTAFDEPQLSSITLTYDTGSGSGS |
| | maturated | FRSPVGDDLNTGHYILGKFSKGPKQQLWGINSNIDQSSHCTITVR |
| | humanized | DGVSVSMSSPSQTMQIDSGGGSSGGGGSGGGGSSVTVLTGQGW |
| | 806 scFV | YPFGRSATVCYYTATDAATVSNLKLFFQNKSTDRSITIRSKLSPQY |
| | VL > VH | RTNGNYSIYGMWELGKGPPQRIWNWAFDRSISYGSVTCTLSLTQ |
| | | SPKVLGPGSEQLQVE |
| 48 | Linker | GGGGSGGGGSSGGGS |
| 49 | Linker | GGTGGCGGAGGATCTGGCGGAGGCGGATCTTCTGGCGGTGGC |
| | | TCT |
| 64 | Chimeric 806 | ATGGCCTTACCAGTGACCGCCTTGCTCCTGCCGCTGGCCTTGC |
| | CAR | TGCTCCACGCCGCCAGGCCGGATGTCCAGCTGCAAGAGTCTGG |
| | | CCCTAGCCTGGTCAAGCCTAGCCAGAGCCTGAGCCTGACATGT |
| | | ACCGTGACCGGCTACAGCATCACCAGCGACTTCGCCTGGAACT |
| | | GGATCAGACAGTTCCCCGGCAACAAGCTGGAATGGATGGGCT |
| | | ACATCAGCTACAGCGGCAACACCCGGTACAACCCCAGCCTGA |
| | | AGTCCCGGATCTCCATCACCAGAGACACCAGCAAGAACCAGT |
| | | TCTTCCTGCAGCTGAACAGCGTGACCATCGAGGACACCGCCAC |
| | | CTACTACTGTGTGACAGCCGGCAGAGGCTTCCCTTATTGGGGA |
| | | CAGGGAACCCTGGTCACAGTGTCTGCTGGTGGCGGAGGATCT |
| | | GGCGGAGGCGGATCTTCTGGCGGTGGCTCTGATATCCTGATGA |
| | | CACAGAGCCCCAGCAGCATGTCTGTGTCCCTGGGCGATACCGT |
| | | GTCCATCACCTGTCACAGCAGCCAGGACATCAACAGCAACAT |
| | | CGGCTGGCTGCAGCAGAGGCCTGGCAAGTCTTTTAAGGGCCTG |
| | | ATCTACCACGGCACCAACCTGGATGATGAGGTGCCCAGCAGA |
| | | TTTTCCGGCTCTGGAAGCGGAGCCGACTACTCCCTGACAATCA |
| | | GCAGCCTGGAAAGCGAGGACTTCGCCGATTACTACTGCGTGC |
| | | AGTACGCCCAGTTTCCTTGGACCTTTGGAGGCGGCACAAAGCT |
| | | GGAAATCAAGCGGACCACGACGCCAGCGCCGCGACCACCAAC |
| | | ACCGGCGCCCACCATCGCGTCGCAGCCCCTGTCCCTGCGCCCA |
| | | GAGGCGTGCCGGCCAGCGGCGGGGGGCGCAGTGCACACGAGG |
| | | GGGCTGGACTTCGCCTGTGATATCTACATCTGGGCCCCTCTGG |
| | | CCGGCACCTGTGGCGTGCTGCTGCTGTCCCTGGTCATCACCCT |
| | | GTACTGCAAGCGGGGCAGAAAGAAGCTGCTGTACATCTTCAA |
| | | GCAGCCCTTCATGCGGCCTGTGCAGACCACACAGGAAGAGGA |
| | | CGGCTGTAGCTGTAGATTCCCCGAGGAAGAGGAAGGCGGCTG |
| | | CGAGCTGAGAGTGAAGTTCAGCAGAAGCGCCGACGCCCCTGC |
| | | CTATCAGCAGGGCCAGAACCAGCTGTACAACGAGCTGAACCT |
| | | GGGCAGACGGGAGGAATACGACGTGCTGGACAAGAGAAGAG |
| | | GCCGGGACCCTGAGATGGGCGGCAAGCCCAGACGGAAGAACC |
| | | CCCAGGAAGGCCTGTATAACGAACTGCAGAAAGACAAGATGG |
| | | CCGAGGCCTACAGCGAGATCGGCATGAAGGGCGAGCGGAGAA |
| | | GAGGCAAGGGCCATGACGGCCTGTACCAGGGCCTGAGCACCG |
| | | CCACCAAGGACACCTACGACGCCCTGCACATGCAGGCCCTGC |
| | | CTCCAAGA |
| 65 | Chimeric 806 | MALPVTALLLPLALLLHAARPDVQLQESGPSLVKPSQSLSLTCTV |
| | CAR | TGYSITSDFAWNWIRQFPGNKLEWMGYISYSGNTRYNPSLKSRISI |
| | | TRDTSKNQFFLQLNSVTIEDTATYYCVTAGRGFPYWGQGTLVTV |
| | | SAGGGGSGGGGSSGGGSDILMTQSPSSMSVSLGDTVSITCHSSQDI |
| | | NSNIGWLQQRPGKSFKGLIYHGTNLDDEVPSRFSGSGSGADYSLT |
| | | ISSLESEDFADYYCVQYAQFPWTFGGGTKLEIRRTTTPAPRPPTPA |
| | | PTIASQYLSLRPEACRPAAGGAVHTRGLDFACDIYIWAPLAGTCG |
| | | VLLLSVITLYCKRGRKKLLYIFKQPFMRPVQTTQEEDGCSCRFPE |
| | | EEEGGCELRVKFSRSADAPAYQQGQNQLYNELNLGRREEYDVL |
| | | DKRRGRDPEMGGKPRRKNPQEGLYNELQKDKMAEAYSEIGMKG |
| | | ERRRGKGHDGLYQGLSTATKDTYDALHMQALPPR |
| 66 | Humanized | ATGGCCTTACCAGTGACCGCCTTGCTCCTGCCGCTGGCCTTGC |
| | 806 CAR | TGCTCCACGCCGCCAGGCCGCAGGTTCAGCTGCAAGAGTCTGG |
| | | CCCTGGCCTGGTCAAGCCTAGCCAAACACTGAGCCTGACCTGT |
| | | ACCGTGTCCGGCTACAGCATCAGCAGCGACTTCGCCTGGAACT |
| | | GGATCAGACAGCCTCCTGGCAAAGGACTGGAATGGATGGGCT |
| | | ACATCAGCTACAGCGGCAACACCAGATACCAGCCTAGCCTGA |
| | | AGTCCCGGATCACCATCAGCAGAGACACCAGCAAGAACCAGT |
| | | TCTTCCTGAAGCTGAACAGCGTGACAGCCGCCGATACCGCCAC |
| | | CTACTATTGTGTGACAGCTGGCAGAGGCTTCCCCTATTGGGGA |
| | | CAGGGAACACTGGTCACCGTTAGCTCTGGTGGCGGAGGATCT |
| | | GGCGGAGGCGGATCTTCTGGCGGTGGCTCTGATATCCAGATGA |
| | | CACAGAGCCCCAGCAGCATGTCCGTGTCCGTGGGAGACAGAG |
| | | TGACCATCACCTGTCACAGCAGCCAGGACATCAACAGCAACA |
| | | TCGGCTGGCTGCAGCAGAAGCCCGGCAAGTCTTTTAAGGGCCT |
| | | GATCTACCACGGCACCAACCTGGATGATGGCGTGCCCAGCAG |
| | | ATTTTCTGGCAGCGGCTCTGGCACCGACTACACCCTGACCATA |
| | | TCTAGCCTGCAGCCTGAGGACTTCGCCACCTATTACTGCGTGC |
| | | AGTACGCCCAGTTTCCTTGGACCTTTGGAGGCGGCACAAAGCT |
| | | GGAAATCAAGCGGACCACGACGCCAGCGCCGCGACCACCAAC |
| | | ACCGGCGCCCACCATCGCGTCGCAGCCCCTGTCCCTGCGCCCA |
| | | GAGGCGTGCCGGCCAGCGGCGGGGGGCGCAGTGCACACGAGG |
| | | GGGCTGGACTTCGCCTGTGATATCTACATCTGGGCCCCTCTGG |
| | | CCGGCACCTGTGGCGTGCTGCTGCTGTCCCTGGTCATCACCCT |
| | | GTACTGCAAGCGGGGCAGAAAGAAGCTGCTGTACATCTTCAA |
| | | GCAGCCCTTCATGCGGCCTGTGCAGACCACACAGGAAGAGGA |
| | | CGGCTGTAGCTGTAGATTCCCCGAGGAAGAGGAAGGCGGCTG |
| | | CGAGCTGAGAGTGAAGTTCAGCAGAAGCGCCGACGCCCCTGC |
| | | CTATCAGCAGGGCCAGAACCAGCTGTACAACGAGCTGAACCT |
| | | GGGCAGACGGGAGGAATACGACGTGCTGGACAAGAGAAGAG |
| | | GCCGGGACCCTGAGATGGGCGGCAAGCCCAGACGGAAGAACC |
| | | CCCAGGAAGGCCTGTATAACGAACTGCAGAAAGACAAGATGG |
| | | CCGAGGCCTACAGCGAGATCGGCATGAAGGGCGAGCGGAGAA |
| | | GAGGCAAGGGCCATGACGGCCTGTACCAGGGCCTGAGCACCG |
| | | CCACCAAGGACACCTACGACGCCCTGCACATGCAGGCCCTGC |
| | | CTCCAAGA |
| 67 | Humanized | MALPVTALLLPLALLLHAARPQVQLQESGPGLVKPSQTLSLTCTV |
| | 806 CAR | SGYSISSDFAWNWIRQPPGKGLEWMGYISYSGNTRYQPSLKSRITI |
| | | SRDTSKNQFFLKLNSVTAADTATYYCVTAGRGFPYWGQGTLVT |
| | | VSSGGGGSGGGGSSGGGSDIQMTQSPSSMSVSVGDRVTITCHSSQ |
| | | DINSNIGWLQQKPGKSFKGLIYHGTNLDDGVPSRFSGSGSGTDYT |
| | | LTISSLQPEDFATYYCVQYAQFPWTFGGGTKLEIKRTTTPAPRPPT |
| | | PAPTIASQPLSLRPEACRPAAGGAVHTRGLDFACDIYIWAPLAGT |
| | | CGVLLLSLVITLYCKRGRKKLLYIFKQPFMRPVQTTQEEDGCSCR |
| | | FPEEEEGGCELRVKFSRSADAPAYQQGQNQLYNELNLGRREEYD |
| | | VLDKRRGRDPEMGGKPRRKNPQEGLYNELQKDKMAEAYSEIGM |
| | | KGERRRGKGHDGLYQGLSTATKDTYDALHMQALPPR |
| 68 | Affinity | ATGGCCTTACCAGTGACCGCCTTGCTCCTGCCGCTGGCCTTGC |
| | maturated | TGCTCCACGCCGCCAGGCCGGAGGTTCAGCTGCAAGAGTCTG |
| | humanized | GCCCTGGCCTGGTCAAGCCTAGCCAAACACTGAGCCTGACCTG |
| | 806 CAR | TACCGTGTCCGGCTACAGCATCAGCAGAGACTTCGCCTGGAAC |
| | | TGGATCAGACAGCCTCCTGGCAAAGGACTGGAATGGATGGGC |
| | | TACATCAGCTACAACGGCAACACCAGATACCAGCCTAGCCTG |
| | | AAGTCCCGGATCACCATCTCCAGAGACACCAGCAAGAACCAG |
| | | TTCTTCCTGAAGCTGAACAGCGTGACAGCCGCCGATACCGCCA |
| | | CCTACTATTGTGTGACAGCCAGCAGAGGCTTCCCCTATTGGGG |
| | | ACAGGGAACCCTGGTCACAGTTAGCTCTGGTGGCGGAGGATC |
| | | TGGCGGAGGCGGATCTTCTGGCGGTGGCTCTGATATCCAGATG |
| | | ACACAGAGCCCCAGCAGCATGTCCGTGTCCGTGGGAGACAGA |
| | | GTGACCATCACCTGTCACAGCAGCCAGGACATCAACAGCAAC |
| | | ATCGGCTGGCTGCAGCAGAAGCCCGGCAAGTCTTTTAAGGGC |
| | | CTGATCTACCACGGCACCAACCTGGATGATGGCGTGCCCAGCA |
| | | GATTTTCTGGCAGCGGCTCTGGCACCGACTACACCCTGACCAT |
| | | ATCTAGCCTGCAGCCTGAGGACTTCGCCACCTATTACTGCGTG |
| | | CAGTACGCCCAGTTTCCTTGGACCTTTGGAGGCGGCACAAAGC |
| | | TGGAAATCAAGACCACGACGCCAGCGCCGCGACCACCAACAC |
| | | CGGCGCCCACCATCGCGTCGCAGCCCCTGTCCCTGCGCCCAGA |
| | | GGCGTGCCGGCCAGCGGCGGGGGGCGCAGTGCACACGAGGGG |
| | | GCTGGACTTCGCCTGTGATATCTACATCTGGGCCCCTCTGGCC |
| | | GGCACCTGTGGCGTGCTGCTGCTGTCCCTGGTCATCACCCTGT |
| | | ACTGCAAGCGGGGCAGAAAGAAGCTGCTGTACATCTTCAAGC |
| | | AGCCCTTCATGCGGCCTGTGCAGACCACACAGGAAGAGGACG |
| | | GCTGTAGCTGTAGATTCCCCGAGGAAGAGGAAGGCGGCTGCG |
| | | AGCTGAGAGTGAAGTTCAGCAGAAGCGCCGACGCCCCTGCCT |
| | | ATCAGCAGGGCCAGAACCAGCTGTACAACGAGCTGAACCTGG |
| | | GCAGACGGGAGGAATACGACGTGCTGGACAAGAGAAGAGGC |
| | | CGGGACCCTGAGATGGGCGGCAAGCCCAGACGGAAGAACCCC |
| | | CAGGAAGGCCTGTATAACGAACTGCAGAAAGACAAGATGGCC |
| | | GAGGCCTACAGCGAGATCGGCATGAAGGGCGAGCGGAGAAG |
| | | AGGCAAGGGCCATGACGGCCTGTACCAGGGCCTGAGCACCGC |
| | | CACCAAGGACACCTACGACGCCCTGCACATGCAGGCCCTGCCT |
| | | CCAAGA |
| 69 | Affinity | MALPVTALLLPLALLLHAARPEVQLQESGPGLVKPSQTLSLTCTV |
| | maturated | SGYSISRDFAWNWIRQPPGKGLEWMGYISYNGNTRYQPSLKSRIT |
| | humanized | ISRDTSKNQFFLKLNSVTAADTATYYCVTASRGFPYWGQGTLVT |
| | 806 CAR | VSSGGGGSGGGGSSGGGSDIQMTQSPSSMSVSVGDRVTITCHSSQ |
| | | DINSNIGWLQQKPGKSFKGLIYHGTNLDDGVPSRFSGSGSGTDYT |
| | | LTISSLQPEDFATYYCVQYAQFPWTFGGGTKLEIKTTTPAPRPPTP |
| | | APTIASQPLSLRPEACRPAAGGAVHTRGLDFACDIYIWAPLAGTC |
| | | GVLLLSLVITLYCKRGRKKLLYIFKQPFMRPVQTTQEEDGCSCRF |
| | | PEEEEGGCELRVKFSRSADAPAYQQGQNQLYNELNLGRREEYDV |
| | | LDKRRGRDPEMGGKPRRKNPQEGLYNELQKDKMAEAYSEIGMK |
| | | GERRRGKGHDGLYQGLSTATKDTYDALHMQALPPR |

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g. a tumor associated antigen) on a target cell (e.g. a cancer cell). The target antigen may include any type of protein, or epitope thereof, associated with the target cell. For example, the CAR may comprise affinity to a target antigen on a target cell that indicates a particular status of the target cell.

In certain embodiments, the CAR of the invention comprises an antigen binding domain that is capable of binding multiple isoforms of epidermal growth factor receptor (EGFR). In certain embodiments, the antigen binding domain is cross-reactive with multiple isoforms of EGFR. In certain embodiments, the antigen binding domain is specifically binds multiple isoforms of EGFR. In certain embodiments, the antigen binding domain of the invention comprises an antibody or fragment thereof, that is capable of binding to multiple EGFR molecules. Preferably, the antigen binding domain is an scFv antibody that is capable of binding to multiple EGFR isoforms. EGFR isoforms include, but are not limited to, wild-type EGFR (wtEGFR), mutated EGFR, EGFR^{A289V}, and EGFR^{R108K}. In certain embodiments, the subject CAR is capable of binding one or more of the EGFR isoforms selected from the group consisting of wild-type EGFR (wtEGFR), mutated EGFR, EGFR^{A289V}, EGFR^{A289D}, EGFR^{289T}, EGFR^{R108K}, EGFR^{R108G}, EGFR^{G598V}, EGFR^{R108K/A289V}, EGFR^{R108k/D126Y}, EGFR^{A289V/G598V}, EGFR^{A289V/C628F}, and EGFR variant II.

In certain embodiments, the antigen binding domain of the CAR binds a dimerized EGFR. For example, the CAR is capable of binding an EGFR homodimer comprising two of the same EGFR isoforms. The CAR is also capable of binding an EGFR heterodimer comprising two different EGFR isoforms, or an oligomer comprising multiple copies of the same or different EGFR isoforms. In certain embodiments, the CAR is capable of binding an EGFR/ErbB heterodimer or oligomer.

As described herein, a CAR of the present disclosure having affinity for a specific target antigen (e.g. EGFR) on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin. In an exemplary embodiment, a CAR of the present disclosure having affinity for EGFR on a target cell may comprise an EGFR binding domain. In some embodiments, the EGFR binding domain is a murine EGFR binding domain, e.g., the EGFR binding domain is of murine origin. In some embodiments, the EGFR binding domain is a humanized EGFR binding domain. In some embodiments, the EGFR binding domain is a human EGFR binding domain, e.g., the EGFR binding domain is of human origin.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof In another embodiment, the antigen binding domain of the CAR is selected from the group consisting of an anti-EGFR antibody or a fragment thereof In some embodiments, the antigen binding domain is selected from the group consisting of an antibody, an antigen binding fragment (Fab), and a single-chain variable fragment (scFv). In some embodiments, an EGFR binding domain of the present invention is selected from the group consisting of an EGFR—specific antibody, an EGFR-specific Fab, and an EGFR-specific scFv. In one embodiment, an EGFR binding domain is an EGFR-specific antibody. In one embodiment, an EGFR binding domain is an EGFR-specific Fab. In one embodiment, an EGFR binding domain is an EGFR-specific scFv.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., Tn-MUC1 binding domain) comprises an scFv having the configuration from N-terminus to C-terminus, VH—linker—VL. In some embodiments, the antigen binding domain (e.g., Tn-MUC1 binding domain) comprises an scFv having the configuration from N-terminus to C-terminus, VL—linker—VH. Those of skill in the art would be able to select the appropriate configuration for use in the present invention.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 36), (GGGS)ₙ (SEQ ID NO: 37), and (GGGGS)ₙ(SEQ ID NO: 38), where n represents an integer of at least 1. Exemplary linker sequences can comprise amino acid sequences including, without limitation, GGSG (SEQ ID NO: 39), GGSGG (SEQ ID NO: 40), GSGSG (SEQ ID NO: 41), GSGGG (SEQ ID NO: 42), GGGSG (SEQ ID NO: 43), GSSSG (SEQ ID NO: 44), GGGGS (SEQ ID NO: 45), GGGGSGGGGSGGGGS (SEQ ID NO: 46) and the like. Those of skill in the art would be able to select the appropriate linker sequence for use in the present invention. In one embodiment, an antigen binding domain (e.g., EGFR binding domain) of the present invention comprises a heavy chain variable region (VH) and a light chain variable region (VL), wherein the VH and VL is separated by the linker sequence having the amino acid sequence GGGGSGGGGSGGGGS (SEQ ID NO: 46), which is encoded by the nucleic acid sequence ggtggcggtggctcgggcggtggtgggtcgggt ggcggcggatct (SEQ ID NO: 47). In certain embodiments, the linker comprises the amino acid sequence of SEQ ID NO: 48. In certain embodiments, the linker is encoded by the nucleic acid sequence of SEQ ID NO: 49.

As used herein, “Fab′” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some instances, the antigen binding domain may be derived from the same species in which the CAR will ultimately be used. For example, for use in humans, the antigen binding domain of the CAR may comprise a human antibody as described elsewhere herein, or a fragment thereof. In some embodiments, a non-human antibody is humanized, where specific sequences or regions of the antibody are modified to increase similarity to an antibody naturally produced in a human. In one embodiment, the antigen binding domain portion is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized chimeric antibodies, substantially less than an intact human variable domain has been substituted by the corresponding sequence from a nonhuman species. In practice, humanized antibodies are typically human antibodies in which some CDR residues and possibly some framework (FR) residues are substituted by residues from analogous sites in rodent antibodies.

In certain embodiments, a CAR of the present invention comprises an EGFR binding domain that is capable of binding multiple EGFR isoforms e.g., an EGFR-specific scFv. In certain embodiments, the antigen binding domain comprises an antibody or fragment thereof derived from the monoclonal antibody mAb 806 (Binder et al. (2018) Cancer cell, 34(1), pp.163-1′7′7). In certain embodiments, the EGFR binding domain comprises the amino acid sequence set forth in SEQ ID NO: 2. In certain embodiments, the EGFR binding domain comprises the amino acid sequence set forth in SEQ ID NO: 32. In certain embodiments, the EGFR binding domain is encoded by the nucleotide sequence set forth in SEQ ID NO: 1. In certain embodiments, the EGFR binding domain is encoded by the nucleotide sequence set forth in SEQ ID NO: 31.

In certain embodiments, the CAR of the present invention comprises an EGFR binding domain that is capable of binding multiple EGFR that is humanized. In certain embodiments, the antigen binding domain comprises an antibody or fragment thereof derived from the monoclonal antibody mAb 806 that has been humanized. In certain embodiments, the EGFR binding domain generally comprises the amino acid sequence set forth in SEQ ID NO: 28. In certain embodiments, the EGFR binding domain comprises the amino acid sequence set forth in SEQ ID NO: 80. In certain embodiments, the EGFR binding domain comprises the amino acid sequence set forth in SEQ ID NO: 82. In certain embodiments, the EGFR binding domain is generally encoded by the nucleotide sequence set forth in SEQ ID NO: 25. In certain embodiments, the EGFR binding domain is encoded by the nucleotide sequence set forth in SEQ ID NO: 79. In certain embodiments, the EGFR binding domain is encoded by the nucleotide sequence set forth in SEQ ID NO: 81.

In certain embodiments, the CAR of the present invention comprises an EGFR binding domain that is capable of binding multiple EGFR that is affinity maturated and humanized. In certain embodiments, the antigen binding domain comprises an antibody or fragment thereof derived from the monoclonal antibody mAb 806 that has been affinity maturated and humanized. In certain embodiments, the EGFR binding domain comprises the amino acid sequence set forth in SEQ ID NO: 84. In certain embodiments, the EGFR binding domain comprises the amino acid sequence set forth in SEQ ID NO: 86. In certain embodiments, the EGFR binding domain is encoded by the nucleotide sequence set forth in SEQ ID NO: 83. In certain embodiments, the EGFR binding domain is encoded by the nucleotide sequence set forth in SEQ ID NO: 85.

In certain embodiments, the antigen binding domain comprises a light chain variable region (VL) comprising the amino acid sequence set forth in SEQ ID NO: 3, which is encoded by SEQ ID NO: 33. The light chain variable region of the antigen binding domain comprises three light chain complementarity-determining regions (CDRs). As used herein, a “complementarity-determining region” or “CDR” refers to a region of the variable chain of an antigen binding molecule that binds to a specific antigen. Accordingly, an EGFR binding domain may comprise a light chain variable region that comprises a CDR1 comprising an amino acid sequence set forth in SEQ ID NO: 5; a CDR2 comprising an amino acid sequence set forth in SEQ ID NO: 6; and a CDR3 comprising an amino acid sequence set forth in SEQ ID NO: 7.

In certain embodiments, the antigen binding domain comprises a light chain variable region comprising the amino acid sequence set forth in SEQ ID NO: 27, which is encoded by the nucleic acid sequence set forth in SEQ ID NO: 24. In certain embodiments, the antigen binding domain comprises a light chain variable region comprising the amino acid sequence set forth in SEQ ID NO: 30.

In certain embodiments, the antigen binding domain comprises a heavy chain variable region (VH) comprising an amino acid sequence set forth in SEQ ID NO: 4, which is encoded by SEQ ID NO: 34. An EGFR binding domain may comprise a heavy chain variable region that comprises a CDR1 comprising an amino acid sequence set forth in SEQ ID NO: 8; a CDR2 comprising an amino acid sequence set forth in SEQ ID NO: 9; and a CDR3 comprising an amino acid sequence set forth in SEQ ID NO: 10.

In certain embodiments, the antigen binding domain comprises a heavy chain variable region comprising the amino acid sequence set forth in SEQ ID NO: 26 which is encoded by the nucleic acid sequence set forth in SEQ ID NO: 23. In certain embodiments, the antigen binding domain comprises a heavy chain variable region comprising the amino acid sequence set forth in SEQ ID NO: 29. In certain embodiments, the antigen binding domain comprises a VH comprising the amino acid sequence set forth in SEQ ID NO: 62 and/or a VL comprising amino acid sequence set forth in SEQ ID NO: 63.

In certain embodiments, the antigen binding domain comprises a heavy chain variable region (VH) comprising an amino acid sequence selected from the group consisting of SEQ ID NOs: 4, 26, and 29 and/or a light chain variable region (VL) comprising an amino acid sequence selected from the group consisting of SEQ ID NOs: 3, 27, and 30. In certain embodiments, the antigen binding domain comprises a heavy chain variable region (VH) encoded by a nucleotide sequence selected from the group consisting of SEQ ID NOs: 43, 23, and 62 and/or a light chain variable region (VL) encoded by a nucleotide sequence selected from the group consisting of SEQ ID NOs: 33, 24, and 63.

Tolerable variations of the EGFR binding domain will be known to those of skill in the art, while maintaining specific binding to EGFR. For example, in some embodiments the EGFR binding domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 2-10, 26-30, 32, 80, 82, 84, or 86 . In some embodiments the EGFR binding domain is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the nucleic acid sequence set forth in any one of SEQ ID NOs: 1, 23-25, 31, 33, 34, 62-63, 79, 81, 83, or 85.

The antigen binding domain may be operably linked to another domain of the CAR, such as the transmembrane domain or the intracellular domain, both described elsewhere herein. In one embodiment, a nucleic acid encoding the antigen binding domain is operably linked to a nucleic acid encoding a transmembrane domain and a nucleic acid encoding an intracellular domain.

The antigen binding domains described herein, such as the antibody or fragment thereof that binds to EGFR can be combined with any of the transmembrane domains described herein, any of the intracellular domains described herein, or any of the other domains described herein that may be included in the CAR.

### Transmembrane Domain

With respect to the transmembrane domain, the CAR of the present invention (e.g., cross-reactive EFGR CAR) can be designed to comprise a transmembrane domain that connects the antigen binding domain to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane regions of particular use in this invention include, without limitation, transmembrane domains derived from (i.e. comprise at least the transmembrane region(s) of) the alpha, beta or zeta chain of the T-cell receptor, CD2, CD27, CD28, CD3 epsilon, CD45, CD4, CDS, CD7, CD8, CD9, CD16, CD22, CD33, CD37, CD64, CD80, CD86, CD134 (OX-40), CD137 (4-1BB), CD154 (CD4OL), CD278 (ICOS), CD357 (GITR), KIR, Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular signaling domains described herein, or any of the other domains described herein that may be included in a subject CAR.

In some embodiments, the transmembrane domain further comprises a hinge region. A subject CAR of the present invention may also include an hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

For example, hinge regions include glycine polymers (G)ₙ, glycine-serine polymers (including, for example, (GS)ₙ, (GSGGS)ₙ (SEQ ID NO: 36) and (GGGS)ₙ (SEQ ID NO: 37), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO: 39), GGSGG (SEQ ID NO: 40), GSGSG (SEQ ID NO: 41), GSGGG (SEQ ID NO: 42), GGGSG (SEQ ID NO: 43), GSSSG (SEQ ID NO: 44), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO: 50); CPPC (SEQ ID NO: 51); CPEPKSCDTPPPCPR (SEQ ID NO: 52) (see, e.g., Glaser et al., J. Biol. Chem. (2005) 280:41494-41503); ELKTPLGDTTHT (SEQ ID NO: 53); KSCDKTHTCP (SEQ ID NO: 54); KCCVDCP (SEQ ID NO: 55); KYGPPCP (SEQ ID NO: 56); EPKSCDKTHTCPPCP (SEQ ID NO: 57) (human IgG1 hinge); ERKCCVECPPCP (SEQ ID NO: 58) (human IgG2 hinge); ELKTPLGDTTHTCPRCP (SEQ ID NO: 59) (human IgG3 hinge); SPNMVPHAHHAQ (SEQ ID NO: 60) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 61); see, e.g., Yan et al., J. Biol. Chem. (2012) 287: 5891-5897. In one embodiment, the hinge region can comprise an amino acid sequence derived from human CD8, or a variant thereof.

In certain embodiments, the transmembrane domain comprises a CD8α transmembrane domain. In certain embodiments, the transmembrane domain comprises a CD8α hinge domain and a CD8a transmembrane domain. In certain embodiments, a subject CAR comprises a CD8α transmembrane domain encoded by the nucleic acid sequence set forth in SEQ ID NO: 12. In certain embodiments, a subject CAR comprises a hinge domain encoded by the nucleic acid sequence of SEQ ID NO: 11 and a transmembrane domain encoded by the nucleic acid sequence set forth in SEQ ID NO: 12.

In certain embodiments, the transmembrane domain comprises a KIR domain. In certain embodiments, the transmembrane domain comprises KIRK domain.

Tolerable variations of the transmembrane and/or hinge domain will be known to those of skill in the art, while maintaining its intended function. For example, in some embodiments the transmembrane domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 11. For example, in some embodiments the hinge transmembrane domain is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleic acid sequences set forth in SEQ ID NO: 12.

The transmembrane domain may be combined with any hinge domain and/or may comprise one or more transmembrane domains described herein. The transmembrane domains described herein, can be combined with any of the antigen binding domains described herein, any of the costimulatory signaling domains or intracellular domains or cytoplasmic domains described herein, or any of the other domains described herein that may be included in the CAR.

In one embodiment, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

Between the extracellular domain and the transmembrane domain of the CAR, or between the intracellular domain and the transmembrane domain of the CAR, there may be incorporated a spacer domain. As used herein, the term “spacer domain” generally means any oligo- or polypeptide that functions to link the transmembrane domain to, either the extracellular domain or, the intracellular domain in the polypeptide chain. A spacer domain may comprise up to 300 amino acids, e.g., 10 to 100 amino acids, or 25 to 50 amino acids. In some embodiments, the spacer domain may be a short oligo- or polypeptide linker, e.g., between 2 and 10 amino acids in length. For example, glycine-serine doublet provides a particularly suitable linker between the transmembrane domain and the intracellular signaling domain of the subject CAR.

### Intracellular Domain

A subject CAR of the present invention also includes an intracellular domain. The intracellular domain of the CAR is responsible for activation of at least one of the effector functions of the cell in which the CAR is expressed (e.g., immune cell). The intracellular domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

In certain embodiments, the intracellular domain comprises a costimulatory signaling domain. In certain embodiments, the intracellular domain comprises an intracellular signaling domain. In certain embodiments, the intracellular domain comprises a costimulatory signaling domain and an intracellular signaling domain. In certain embodiments, the intracellular domain comprises 4-1BB and CD3 zeta. In certain embodiments, the intracellular domain comprises 4-1BB. In certain embodiments, the intracellular domain comprises CD3 zeta. In certain embodiments, the intracellular domain comprises KIRS2.

In one embodiment, the intracellular domain of the CAR comprises a costimulatory signaling domain which includes any portion of one or more co-stimulatory molecules, such as at least one signaling domain from CD3, CD8, CD27, CD28, ICOS, 4-IBB, PD-1, any derivative or variant thereof, any synthetic sequence thereof that has the same functional capability, and any combination thereof.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ϵ), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma Rlla, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11 a, LFA-1, ITGAM, CD lib, ITGAX, CD11 c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, any KIR, e.g., KIR2, KIRS2, KIR2DS2, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, the intracellular signaling domain includes the signaling domains of human immunoglobulin receptors that contain immunoreceptor tyrosine based activation motifs (ITAMs) such as, but not limited to, FcgammaRl, FcgammaRIIA, FcgammaRllC, FcgammaRIIIA, FcRL5 (see, e.g., Gillis et al., Front. (2014) Immunol. 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in a subject CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CDS, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular domains described herein can be combined with any of the antigen binding domains described herein, any of the transmembrane domains described herein, or any of the other domains described herein that may be included in the CAR.

In certain embodiments, the intracellular domain of a subject CAR comprises a 4-1BB costimulatory domain. In certain embodiments, the intracellular domain is encoded by the nucleic acid sequence set forth in SEQ ID NO: 13. In certain embodiments, the intracellular domain of a subject CAR comprises a CD3 zeta intracellular signaling domain. In certain embodiments, the intracellular domain is encoded by the nucleic acid sequence set forth in SEQ ID NO: 14. In certain embodiments, the intracellular domain of a subject CAR comprises a 4-1BB domain and a CD3 zeta domain. In certain embodiments, the intracellular domains is encoded by a nucleic acid sequence comprising SEQ ID NO: 13 and SEQ ID NO: 14.

In certain embodiments, the intracellular domain comprises a DAP12 domain. In certain embodiments, the intracellular domain comprises a MR domain. The intracellular domain may comprise one or more intracellular domains described herein described herein. In certain embodiments, the CAR comprises a KIRS2 transmembrane and intracellular domain. In certain embodiments, the intracellular domain is encoded by the nucleotide sequence comprising SEQ ID NO: 18.

Tolerable variations of the intracellular domain will be known to those of skill in the art, while maintaining specific activity. For example, in some embodiments the intracellular domain is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleic acid sequences set forth in SEQ ID NOs: 13, 14 and/or 18.

In another embodiment, a spacer domain may be incorporated between the antigen binding domain and the transmembrane domain of the CAR, or between the intracellular domain and the transmembrane domain of the CAR. As used herein, the term “spacer domain” generally means any oligo- or polypeptide that functions to link the transmembrane domain to, either the antigen binding domain or, the intracellular domain in the polypeptide chain. In one embodiment, the spacer domain may comprise up to 300 amino acids, preferably 10 to 100 amino acids and most preferably 25 to 50 amino acids. In another embodiment, a short oligo- or polypeptide linker, preferably between 2 and 10 amino acids in length may form the linkage between the transmembrane domain and the intracellular domain of the CAR. An example of a linker includes a glycine-serine doublet.

KIR-CAR

In certain aspects, the present invention relates to a “KIR-CAR”, which is a CAR design comprising a component of a receptor found on natural killer (NK) cells. The KIR-CAR provided herein comprises an antigen binding domain capable of binding multiple isoforms of epidermal growth factor receptor (EGFR) and a KIR transmembrane domain and/or a KIR intracellular (cytoplasmic) domain.

Accordingly, the invention provides a composition comprising a KIR-CAR, an isolated nucleic acid comprising a KIR-CAR, an isolated polypeptide comprising a KIR-CAR, and recombinant T cells comprising the KIR-CAR; wherein the KIR-CAR comprises an antigen binding domain capable of binding multiple isoforms of EGFR.

NK cells are mononuclear cells that develop in the bone marrow from lymphoid progenitors. Morphological features typically include the expression of the cluster determinants (CDs) CD16, CD56, and/or CD57 and the absence of the alpha/beta or gamma/delta TCR complex on the cell surface. Biological properties typically include the ability to bind to and kill target cells that fail to express “self” major histocompatibility complex (MHC)/human leukocyte antigen (HLA) proteins, and the ability to kill tumor cells or other diseased cells that express ligands for activating NK receptors. NK cells are characterized by their ability to bind and kill several types of tumor cell lines without the need for prior immunization or activation. NK cells can also release soluble proteins and cytokines that exert a regulatory effect on the immune system; and can undergo multiple rounds of cell division and produce daughter cells with similar biologic properties as the parent cell. Upon activation by interferons and/or cytokines, NK cells mediate the lysis of tumor cells and of cells infected with intracellular pathogens by mechanisms that require direct, physical contacts between the NK cell and the target cell. Lysis of target cells involves the release of cytotoxic granules from the NK cell onto the surface of the bound target, and effector proteins such as perforin and granzyme B that penetrate the target plasma membrane and induce apoptosis or programmed cell death. Normal, healthy cells are protected from lysis by NK cells. NK cell activity is regulated by a complex mechanism that involves both stimulating and inhibitory signals.

Briefly, the lytic activity of NK cells is regulated by various cell surface receptors that transduce either positive or negative intracellular signals upon interaction with ligands on the target cell. The balance between positive and negative signals transmitted via these receptors determines whether or not a target cell is lysed (killed) by a NK cell. NK cell stimulatory signals can be mediated by Natural Cytotoxicity Receptors (NCR) such as NKp30, NKp44, and NKp46; as well as NKG2C receptors, NKG2D receptors, certain activating killer cell immunoglobulin-like receptors (KIRs), and other activating NK receptors (Lanier, Annual Review of Immunology 2005; 23:225-74). NK cell inhibitory signals can be mediated by receptors like Ly49, CD94/NKG2A, as well as certain inhibitory KIRs, which recognize major histocompatibility complex (MHC) class I molecules (Kane et al., Nature 1986; 319:675-8; Ohlen et al, Science 1989; 246:666-8). These inhibitory receptors bind to polymorphic determinants of MHC class I molecules (including HLA class I) present on other cells and inhibit NK cell-mediated lysis.

KIRs, referred to as killer cell immunoglobulin-like receptors, have been characterized in humans and non-human primates, and are polymorphic type 1 trans-membrane molecules present on certain subsets of lymphocytes, including NK cells and some T cells. KIRs interact with determinants in the alpha 1 and 2 domains of the MHC class I molecules and, as described elsewhere herein, distinct KIRs are either stimulatory or inhibitory for NK cells.

The nomenclature for KIRs is based upon the number of extracellular domains (KIR2D and KIR3D having two and three extracellular Ig-domains, respectively) and whether the cytoplasmic tail is long (KIR2DL or KIR3DL) or short (KIR2DS or KIR3DS). The presence or absence of a given KIR is variable from one NK cell to another within the NK population present in a single individual. Among humans, there is also a relatively high level of polymorphism of KIR genes, with certain KIR genes being present in some, but not all individuals. The expression of KIR alleles on NK cells is stochastically regulated, meaning that, in a given individual, a given lymphocyte may express one, two, or more different KIRs, depending on the genoptype of the individual. The NK cells of a single individual typically express different combinations of KIRs, providing a repertoire of NK cells with different specificities for MHC class I molecules.

Certain KIR gene products cause stimulation of lymphocyte activity when bound to an appropriate ligand. The activating KIRs all have a short cytoplasmic tail with a charged trans-membrane residue that associates with an adapter molecule having an

Immunoreceptor Tyrosine-based Activation Motifs (ITAMs) which transduce stimulatory signals to the NK cell. By contrast, inhibitory KIRs have a long cytoplasmic tail containing Immunoreceptor Tyrosine-based Inhibitory Motif (ITIM), which transduce inhibitory signals to the NK cell upon engagement of their MEW class I ligands. The known inhibitory KIRs include members of the KIR2DL and KIR3DL subfamilies. Inhibitory KIRs having two Ig domains (KIR2DL) recognize HLA-C allotypes: KIR2DL2 (formerly designated p58.2) and the closely related, allelic gene product KIR2DL3 both recognize “group 1” HLA-C allotypes (including HLA-Cw1, −3, −7, and −8), whereas KIR2DL1 (p58.1) recognizes “group 2” HLA-C allotypes (such as HLA-Cw2, −4, −5, and −6). The recognition by KIR2DL1 is dictated by the presence of a Lys residue at position 80 of HLA-C alleles. KIR2DL2 and KIR2DL3 recognition is dictated by the presence of an Asn residue at position 80 in HLA-C. Importantly, the great majority of HLA-C alleles have either an Asn or a Lys residue at position 80. Therefore, KIR2DL1, -2, and -3 collectively recognize essentially all HLA-C allotypes found in humans. One KIR with three Ig domains, KIR3DL1 (p70), recognizes an epitope shared by HLA-Bw4 alleles. Finally, KIR3DL2 (p140), a homodimer of molecules with three Ig domains, recognizes HLA-A3 and -A11.

However, the invention should not be limited to inhibitory KIRs comprising a cytoplasmic tail containing ITIM. Rather, any inhibitory protein having a cytoplasmic domain that is associated with an inhibitory signal can be used in the construction of the CARs of the invention. Non-limiting examples of an inhibitory protein include but are not limited CTLA-4, PD-1, and the like. These proteins are known to inhibit T cell activation.

Accordingly, the invention provides a KIR-CAR comprising an extracellular domain that comprises a target-specific binding element otherwise referred to as an antigen binding domain fused to a KIR or fragment thereof. In one embodiment, the KIR is an activating KIR that comprises a short cytoplasmic tail that associates with an adapter molecule having an Immunoreceptor Tyrosine-based Activation Motifs (ITAMs) which transduce stimulatory signals to the NK cell.

In some instances, it is desirable to remove/ not include a hinge region when constructing a KIR-CAR. Without wishing to be bound by specific theory, removing the hinge region of the KIR-CAR may result in increased cytolytic activity.

In certain embodiments, the invention provides an isolated nucleic acid comprising an EF1alpha sequence, a DAP12 sequence, a T2A sequence, an 806-scFv sequence, a KIR transmembrane domain sequence, and a KIR cytoplasmic (intracellular) domain sequence.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

In certain embodiments, the invention provides KIR-CAR comprising an antigen binding domain comprising an 806-scFv, a KIR transmembrane domain, and/or a KIR intracellular domain. The KIR-CAR may optionally comprise a DAP12 domain, or the KIR-CAR may be co-expressed with DAP12.

In certain embodiments, the KIR is selected from the group consisting of KIRS2, KIR2DS2 and KIR2. In certain embodiments, the linker is a short glycine-serine linker.

Also included in the invention is a genetically modified cell comprising a KIR-CAR that is capable of binding multiple isoforms of EGFR. In certain embodiments, the KIR-CAR comprises an 806-scFv, a KIR transmembrane domain, and/or a KIR intracellular domain.

### CAR Sequences

A subject CAR of the present invention is a CAR having affinity for multiple isoforms of EFGR (e.g. wtEGFR, mutated EGFR, EGFR^{A289V}, EGFR^{A289D}, EGFR^{A289T}, EGFR^{R108K}, EGFR^{R108G}, and EGFR^{G598V}). In one embodiment, the EGFR CAR of the present invention comprises the amino acid sequence set forth in SEQ ID NO: 20, which may be encoded by the nucleic acid sequence set forth in SEQ ID NO: 19.

In another embodiment, the CAR comprises the amino acid sequence set forth in SEQ ID NO: 22, which may be encoded by the nucleic acid sequence set forth in SEQ ID NO: 21. In another embodiment, the CAR comprises the amino acid sequence set forth in SEQ ID NO: 65, which may be encoded by the nucleic acid sequence set forth in SEQ ID NO: 64. In another embodiment, the CAR comprises the amino acid sequence set forth in SEQ ID NO: 67, which may be encoded by the nucleic acid sequence set forth in SEQ ID NO: 66. In another embodiment, the CAR comprises the amino acid sequence set forth in SEQ ID NO: 69, which may be encoded by the nucleic acid sequence set forth in SEQ ID NO: 68.

Tolerable variations of the CAR will be known to those of skill in the art, while maintaining specific activity. For example, in some embodiments the CAR comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 22, SEQ ID NO: 20, SEQ ID NO: 65, SEQ ID NO: 67, or SEQ ID NO: 69. For example, in some embodiments the CAR is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the nucleic acid sequence set forth in SEQ ID NO: 19, SEQ ID NO: 21, SEQ ID NO: 64, SEQ ID NO: 66, or SEQ ID NO: 68.

Accordingly, a subject CAR of the present invention comprises an antigen binding domain capable of binding multiple isoforms of EGFR and a transmembrane domain. In one embodiment, the CAR comprises an antigen binding domain capable of binding multiple isoforms of EGFR and a transmembrane domain, wherein the transmembrane domain comprises a CD8 hinge region. In one embodiment, the CAR comprises an antigen binding domain capable of binding multiple isoforms of EGFR and a transmembrane domain, wherein the transmembrane domain comprises a CD8 transmembrane domain. In one embodiment, the CAR comprises an antigen binding domain capable of binding multiple isoforms of EGFR and a transmembrane domain, wherein the transmembrane domain comprises a CD8 hinge region and a CD8 transmembrane domain. In one embodiment, the CAR comprises an antigen binding domain capable of binding multiple isoforms of EGFR and a transmembrane domain, wherein the transmembrane domain comprises a KIR transmembrane domain.

Accordingly, a subject CAR of the present invention comprises an antigen binding domain capable of binding multiple isoforms of EGFR, a transmembrane domain, and an intracellular domain. In one embodiment, the CAR comprises an antigen binding domain capable of binding multiple isoforms of EGFR, a transmembrane domain, and an intracellular domain, wherein the intracellular domain comprises a 4-1BB domain. In one embodiment, the CAR comprises an antigen binding domain capable of binding multiple isoforms of EGFR, a transmembrane domain, and an intracellular domain, wherein the intracellular domain comprises a CD3 zeta domain. In one embodiment, the CAR comprises an antigen binding domain capable of binding multiple isoforms of EGFR, a transmembrane domain, and an intracellular domain, wherein the intracellular domain comprises a 4-1BB domain and a CD3 zeta domain. In one embodiment, the CAR comprises an antigen binding domain capable of binding multiple isoforms of EGFR, a transmembrane domain, and an intracellular domain, wherein the intracellular domain comprises a KIR domain.

Accordingly, the present invention provides a modified immune cell or precursor cell thereof, e.g., a modified T cell, comprising a chimeric antigen receptor (CAR) having affinity for multiple isoforms of EGFR as described herein.

### Methods of Treatment

The modified cells (e.g., T cells comprising CARs capable of binding multiple isoforms of EGFR) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the invention includes a method for treating cancer in a subject in need thereof comprising administering to the subject a composition comprising any of the modified T cells of the present invention.

In another aspect, the invention includes a method of treating cancer in a subject in need thereof comprising: a.) culturing a plurality of CAR T cells with a GBM organoid (GBO), b.) selecting from the plurality of CART cells, a CART cell having the highest efficacy, and c.) administering the CAR T cell with the highest efficacy to the subject, thus treating the cancer in the subject. In certain embodiments, the plurality of CAR T cells comprises a plurality of modified T cells comprising a plurality of CARs, wherein each CAR comprises an antigen binding domain, a transmembrane domain, and an intracellular domain. In certain embodiments, the antigen binding domain is capable of binding an antigen selected from the group consisting of CD19, EGFR, multiple isoforms of EGFR (e.g. wild-type EGFR (wtEGFR), mutated EGFR, EGFRA289V, EGFRA289D, EGFRA289T, EGFRR108K, EGFRR108G, EGFRG598V, EGFRD126Y, EGFRC628F, EGFRR108K/A289V, EGFRR108K/D126Y, EGFRA289V/G598V, EGFRA289V/C628F, and EGFR variant II), PSMA, PSCA, and any tumor associated antigen (TAA). In certain embodiments, GBO is generated from a biopsy from the subject. In such an embodiment, the GBO is specific to the subject and thus the cancer treatment (e.g. choice of CAR) is personalized to that subject. CART treatment can be combined with a secondary treatment (e.g. immune checkpoint blockade (ICB)). A secondary treatment can be administered prior to, during, or after CART treatment.

In certain embodiments, the cancer to be treated is glioblastoma (GBM). In certain embodiments, treating GBM by the methods described herein serves to overcome the intra-tumoral antigenic heterogeneity and/or adaptive resistance of GBM. The modified cells of the invention (e.g., CAR T cells) are capable of binding cells (e.g. cancer/tumor cells) expressing one or more isoforms of EGFR, thus treating a disease or disorder associated with expression of EGFR (e.g. cancer). EGFR isoforms on the cells, which the CAR T cells are capable of binding, include but are not limited to wild-type EGFR (wtEGFR), mutated EGFR, EGFR^{A289V}, EGFR^{A289D}, EGFR^{A289T}, EGFR^{R108K}, EGFR^{R108G}, EGFR^{G598V}, EGFR^{D126V}, EGFR^{C628F}, EGFR^{R108K/A289V}, EGFR^{R108K/D126Y}, EGFR^{A289V/G598V}, EGFR^{A289V/C628F}, and EGFR variant II.

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

The modified immune cells of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited to breast cancer, neck cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a solid tumor or a hematological tumor. In one embodiment, the cancer is a carcinoma. In one embodiment, the cancer is a sarcoma. In one embodiment, the cancer is a leukemia. In one embodiment the cancer is a solid tumor. In one embodiment the cancer is breast cancer. In one embodiment, the cancer is GBM.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art. The cells of the invention to be administered may be autologous, with respect to the subject undergoing therapy.

The administration of the cells and compositions of the invention may be carried out in any convenient manner known to those of skill in the art. The cells and compositions of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, intrathecally, by intravenous (i.v.) injection, or intraperitoneally. In other instances, the cells of the invention are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In certain embodiments, the subject is provided a secondary treatment. Secondary treatments include but are not limited to chemotherapy, radiation, surgery, and medications.

In certain embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cell comprising a CAR may be administered to a subject in combination with an immune checkpoint inhibitor (e.g., an inhibitor of PD-1, CTLA-4, PD-L1, or TIM-3). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). In certain embodiments, the modified cell may be administered in combination with an anti-T-cell inhibitory receptor Tim-3 (T-cell immunoglobulin and mucin-domain containing-3) antibody. Other types of immune checkpoint inhibitors may also be used including, but not limited to, small molecules, siRNA, miRNA, and CRISPR systems. Immune checkpoint inhibitors may be administered before, after, or concurrently with the modified cell comprising the CAR.

In certain embodiments, the CART cells of the present invention can serve as localized delivery vehicles across the blood brain barrier (BBB). “Minibodies”, or blocking proteins, are scFv sequences targeting checkpoint molecules (e.g. PD-1, CTLA-4, TIM-3) combined with a human IgG CH3 region, that can be encoded in the CAR lentivirus. CART cells can be redirected to neoantigens in GBM and locally secrete blocking proteins, thus overcoming the potential limitation of checkpoint blockade molecule uptake in the central nervous system. As T cells have the capacity to efficiently cross the BBB, endowing T cells with the ability to produce a protein therapeutic is a particularly attractive strategy for overcoming otherwise limited drug permeability into the central nervous system. This targeted delivery strategy also reduces the risk of systemic off-target effects of ICB.

### Introduction of Nucleic Acids

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

RNA

In one embodiment, the nucleic acids introduced into the T cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a chimeric membrane protein. By way of example, the template encodes an antibody, a fragment of an antibody or a portion of an antibody. By way of another example, the template comprises an extracellular domain comprising a single chain variable domain of an antibody, such as anti-CD3, and an intracellular domain of a co-stimulatory molecule. In one embodiment, the template for the RNA chimeric membrane protein encodes a chimeric membrane protein comprising an extracellular domain comprising an antigen binding domain derived from an antibody to a co-stimulatory molecule, and an intracellular domain derived from a portion of an intracellular domain of CD28 and 4-1BB.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′

UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA is electroporated into the cells, such as in vitro transcribed RNA.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Sources of T Cells

In certain embodiments, a source of T cells is obtained from a subject. Non-limiting examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, umbilical cord, and tumors. In certain embodiments, any number of T cell lines available in the art, may be used. In certain embodiments, T cells can be obtained from a unit of blood collected from a subject using any number of techniques known to the skilled artisan, such as Ficoll separation. In one embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

### Expansion of T Cells

In certain embodiments, the modified cells disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the modified cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-a. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more by culturing the electroporated population.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### Pharmaceutical Compositions

Pharmaceutical compositions of the present invention may comprise the modified T cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The cells of the invention to be administered may be autologous, allogeneic or xenogeneic with respect to the subject undergoing therapy.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It can generally be stated that a pharmaceutical composition comprising the modified T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the modified T cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Expression of EGFR Missense Mutants in U87 Cells

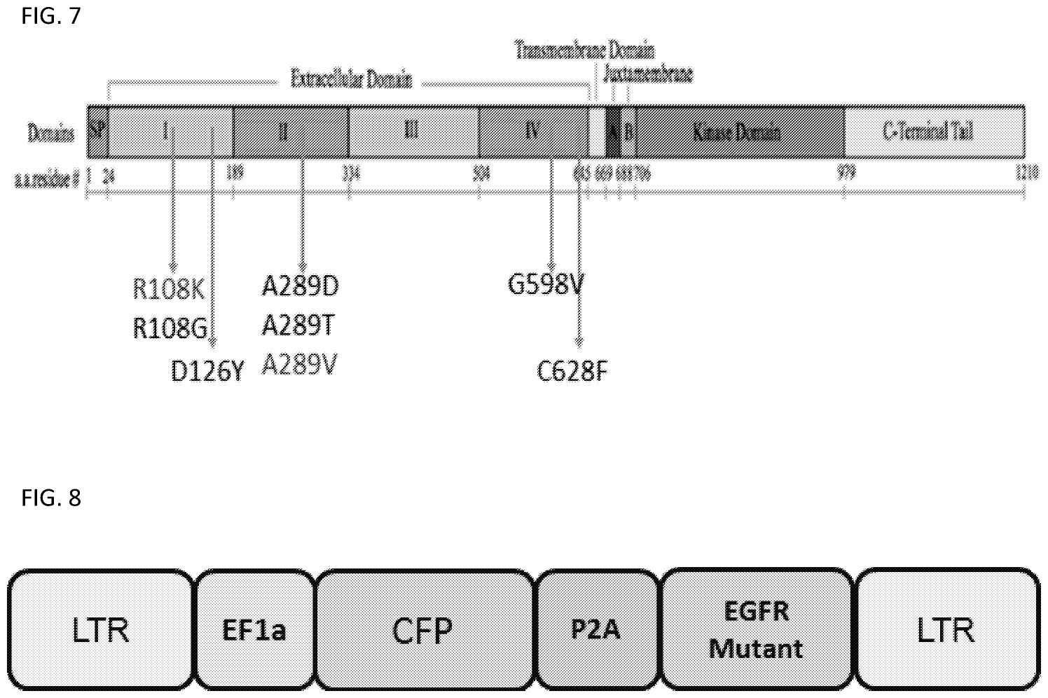

Alterations within the epidermal growth factor receptor (EGFR) (ErbB1) locus represent the most frequent genetic alterations in GBM. EGFR overexpression, such as that mediated through focal amplification of the EGFR locus as double minute chromosomes, has long been recognized in GBM, and is found in 60% of cases. EGFR mutations are also frequent. The oncogenic EGFR variant lacking exons 2-7 (EGFRvIII) is found in approximately 30% of GBM. Using next-generation sequencing data of GBM cases as well as TCGA data, missense mutations with oncogenic activity were identified at positions 108, 289, and 598 of the EGFR extracellular domain (ECD) (FIG. 7). Retrospective analysis showed that patients bearing these mutations demonstrate poor overall survival. Over 60% of EGFR amplified GBMs demonstrated mutations in the ECD that could be targeted by cross-reactive CART cells.

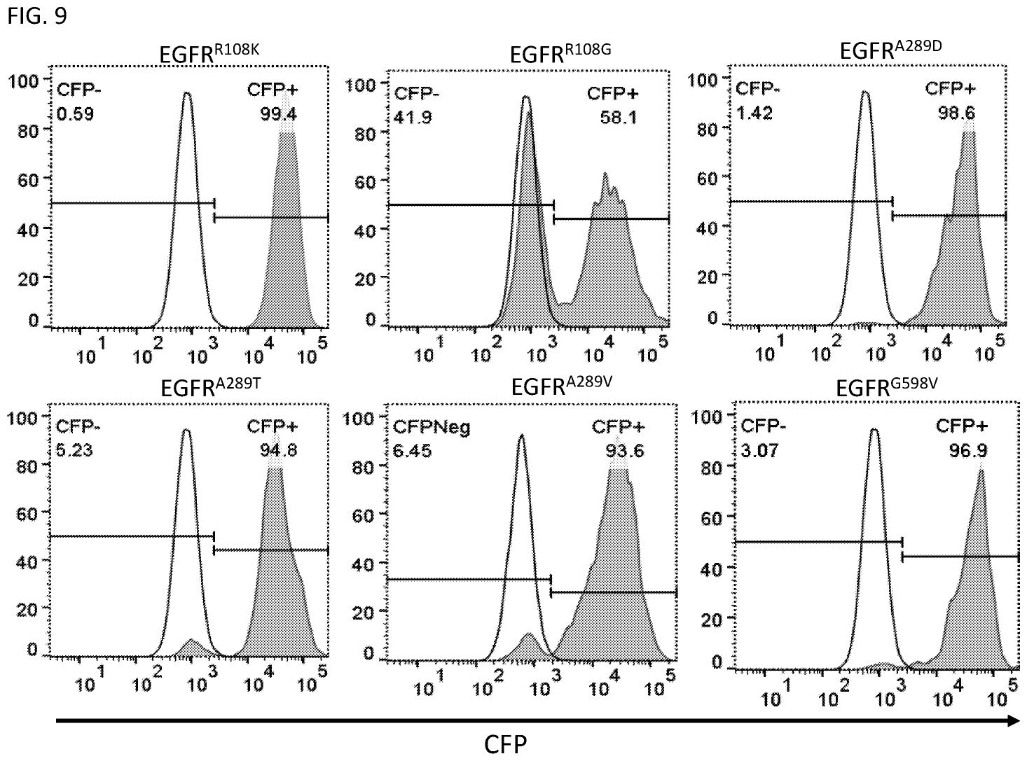

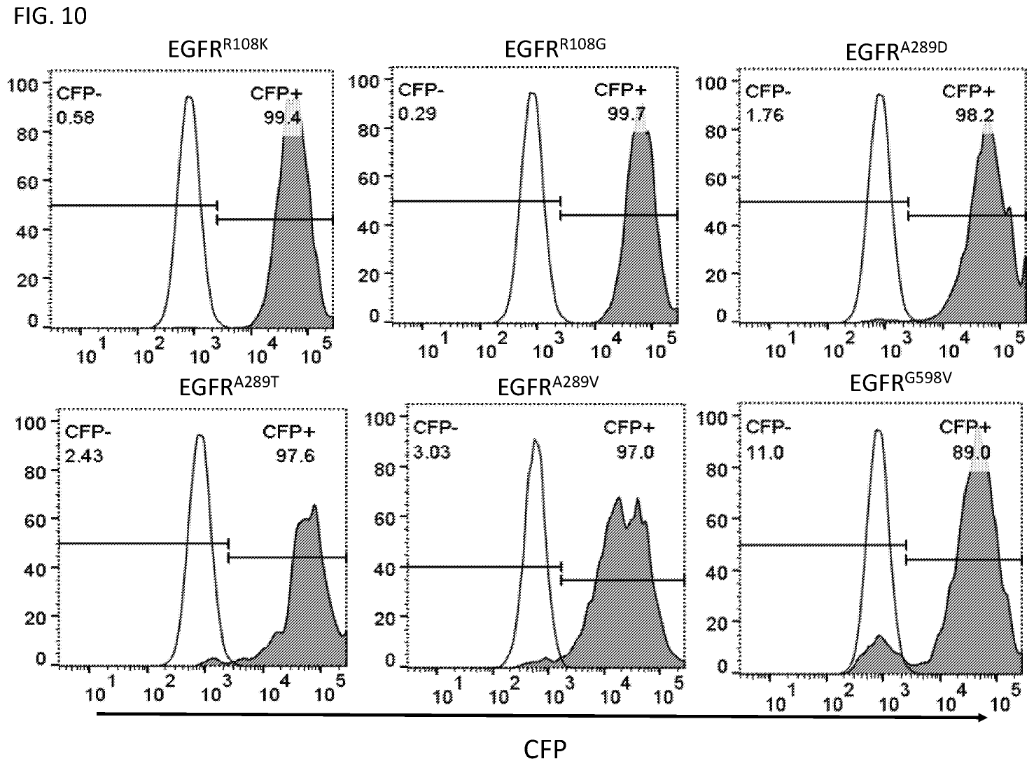

In order to test the function of EGFR-specific CAR T cells, a lentiviral expression system that encodes mutants of EGFR implicated in GBM was generated (FIG. 8). EGFR missense mutations were introduced into the EGFR gene by Geneart gene synthesis and site directed mutagenesis (Thermo fisher). Lentiviral vectors co-expressing CFP and EGFR mutations were transduced into U87 wtEGFR and U87 MG cell lines and CFP positive cells were sorted by fluorescence activated cell sorting. Co-expression of EGFR-mutants and CFP in U87 MG cell line transduced with wtEGFR (U87 wtEGFR) is shown in FIG. 9. Co-expression of EGFR-mutants and CFP in U87 MG GBM cells is shown in FIG. 10.

### Example 2

### Targeted Cell Lysis of GBM Cells by Cross-Reactive EGFR-Specific CAR T cells

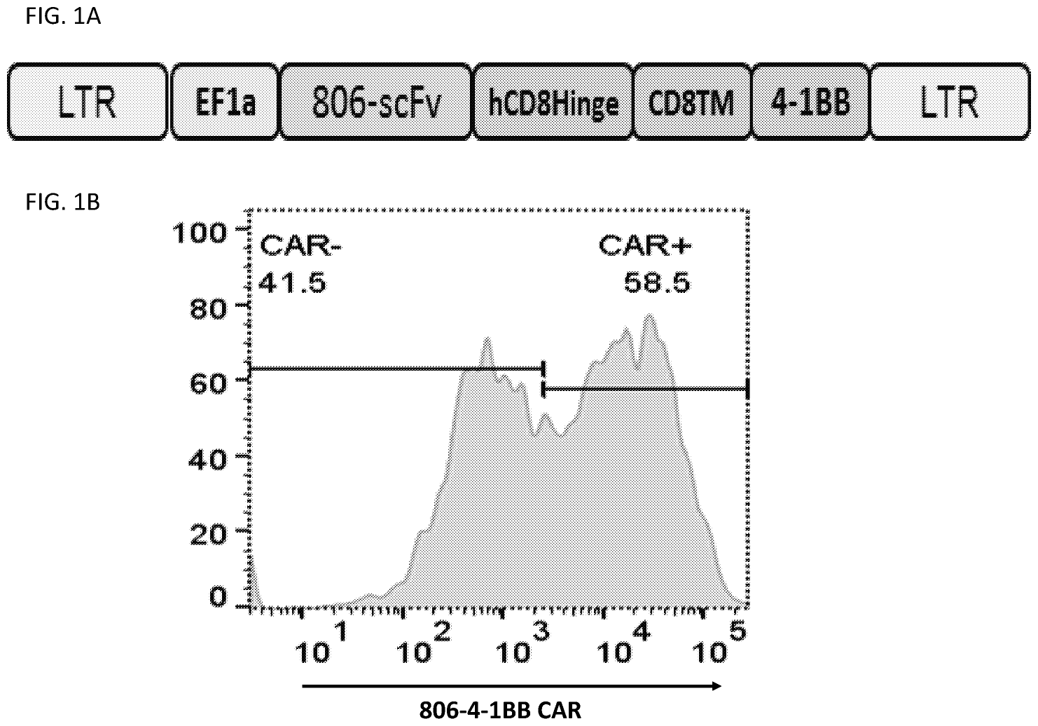

Chimeric antigen receptor (CAR) T cells that elicit broad specificity to multiple EGFR isoforms were generated herein. A lentiviral expression vector was constructed that encodes the scFv from the monoclonal antibody mAb806, a CD8 hinge domain, a CD8 transmembrane domain, and a 4-1BB intracellular signaling domain (FIG. 1A). Primary human CD4+ and CD8+ T cells were transduced with the CAR-encoding lentiviral vector. After 6 days incubation, approximately 60% of T cells expressed the EGFR-specific 806-4-1BB CAR on the cell surface (FIG. 1B).

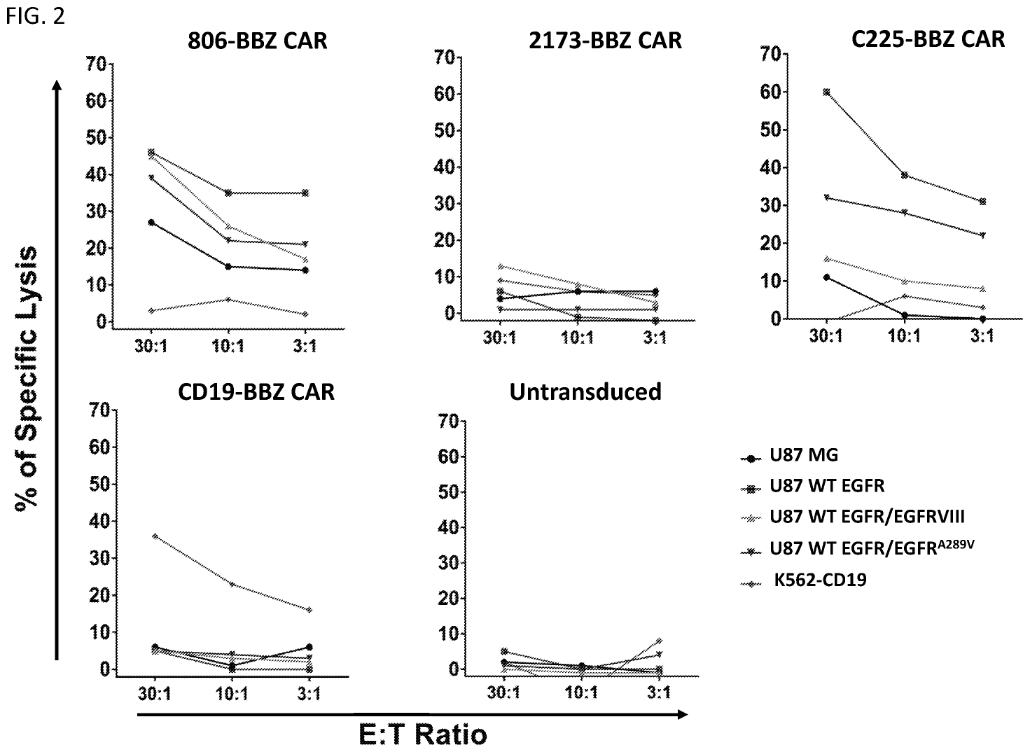

The monoclonal antibody mAb806 detects a structural feature shared by multiple EGFR missense mutations (Binder et al. (2018) Cancer Cell, 34, 1; 163-177). Thus, CAR T cells that incorporate the 806scFv, should be capable of broad specificity and targeting of a heterogeneous EGFR tumor cell population. The antigen specific cytolytic activity of 806-41BB CAR T cells was tested against human GBM cell lines (FIG. 2). GBM cell lines were based on the U87 MG parental GBM cell line that has a basal level of EGFR and were transduced with either wild-type EGFR (U87 wtEGFR) or its variants EGFRvIII (U87 wtEGFR/EGFRvIII) or EGFR^{A289V} (U87 wtEGFR/EGFR^{A289V}). Antigen specific cytolytic activity was measured in a 4 hour chromium release assay using different CAR T cell to tumor cell ratios. The 2173 CAR, which is specific for EGFRvIII, and the Cetuximab (C225) CAR, which is specific for wild type EGFR, were used as positive controls. CD19 CART cells were used as a negative control. 806-41BB CAR T cells demonostrated antigen specific cytolytic activity against multiple EGFR isoforms (FIG. 2).

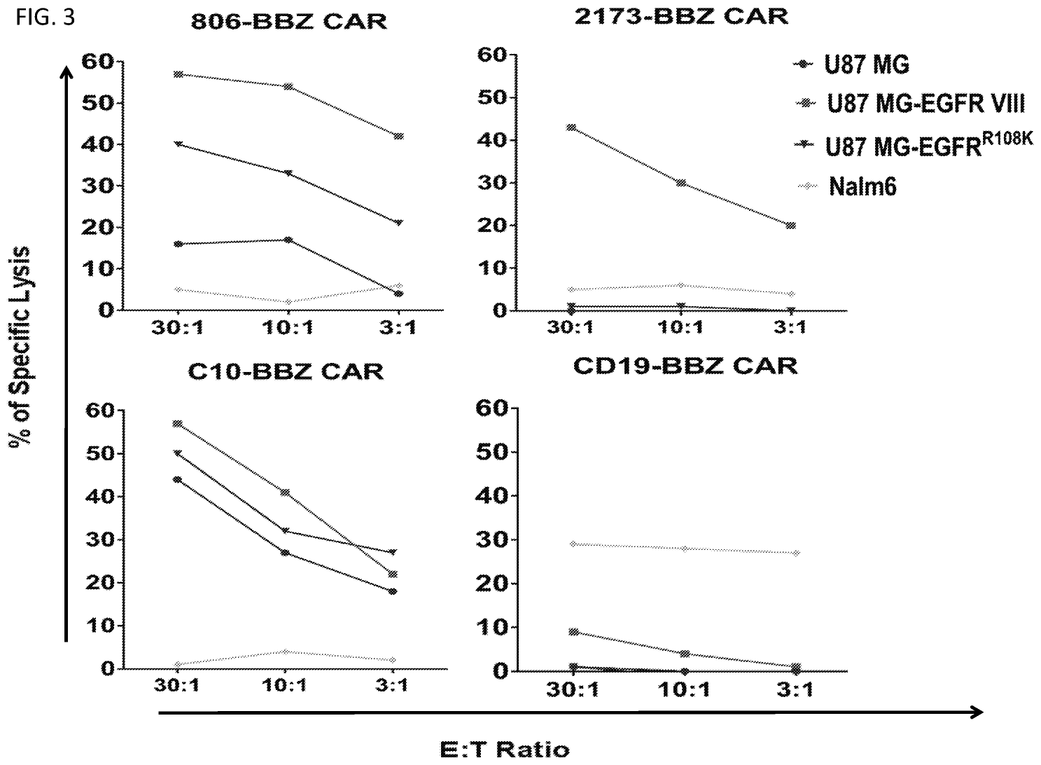

In vitro cytolysis by 806 4-1BB CAR T cells was demonstrated in U87 parental cell lines transduced with EGFR missense mutations R108K and A289V and EGFR variant VIII in 4hr chromium release assay. EGFR wild type specific C10 4-1BB, and VIII-specific 2173, 4-1BB CARs were used as positive controls. CD19 4-1BB CAR was used as negative control. 806 CAR T cells were able to specificaly lyse wild type and mutant EGFR-expressing U87 cells while control Nalm6 cells, a precursor B cell line that does not express EGFR, were not targeted (FIG. 3). Taken together, these data demonstrated that the 806-41BB CAR T cell is able to specifically target and kill a variety of EGFR-expressing cells.

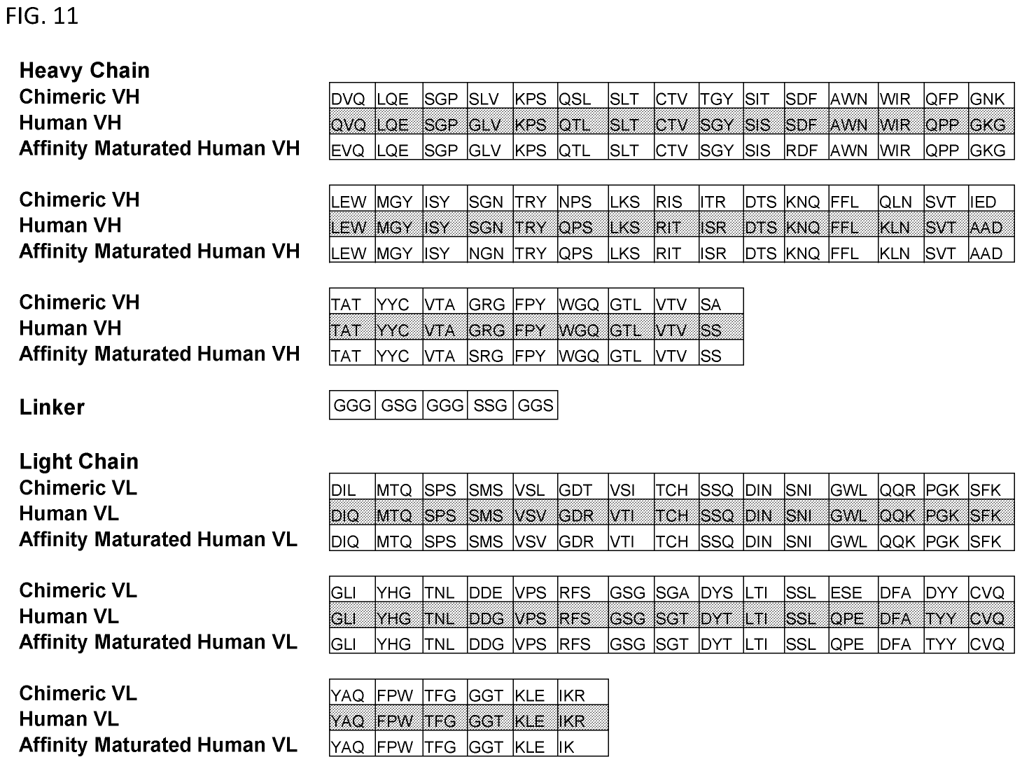

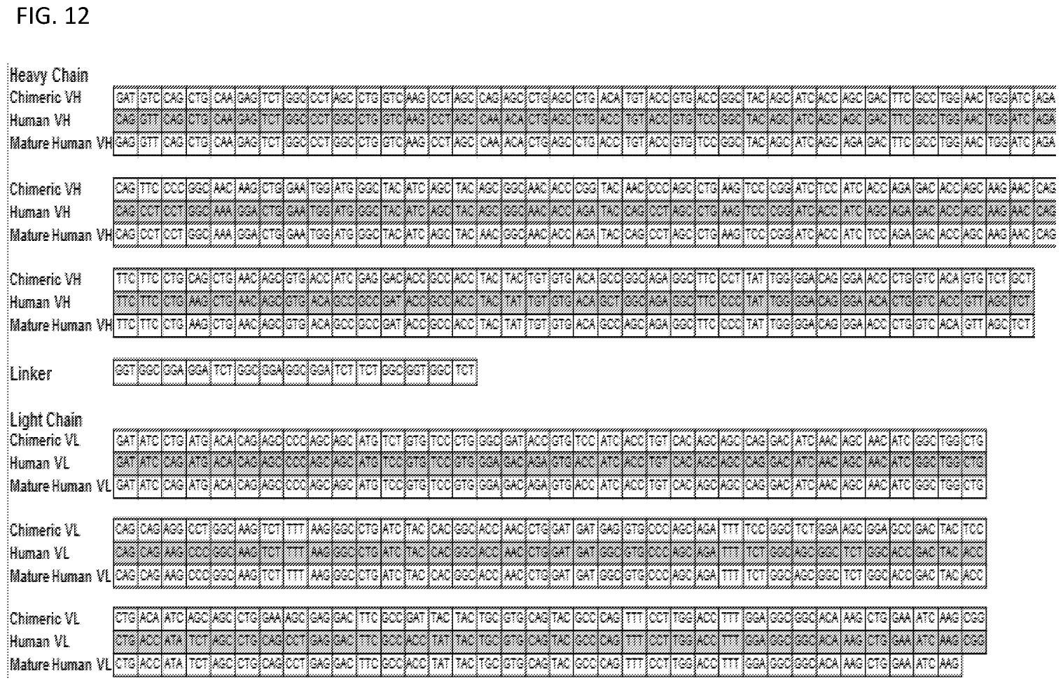

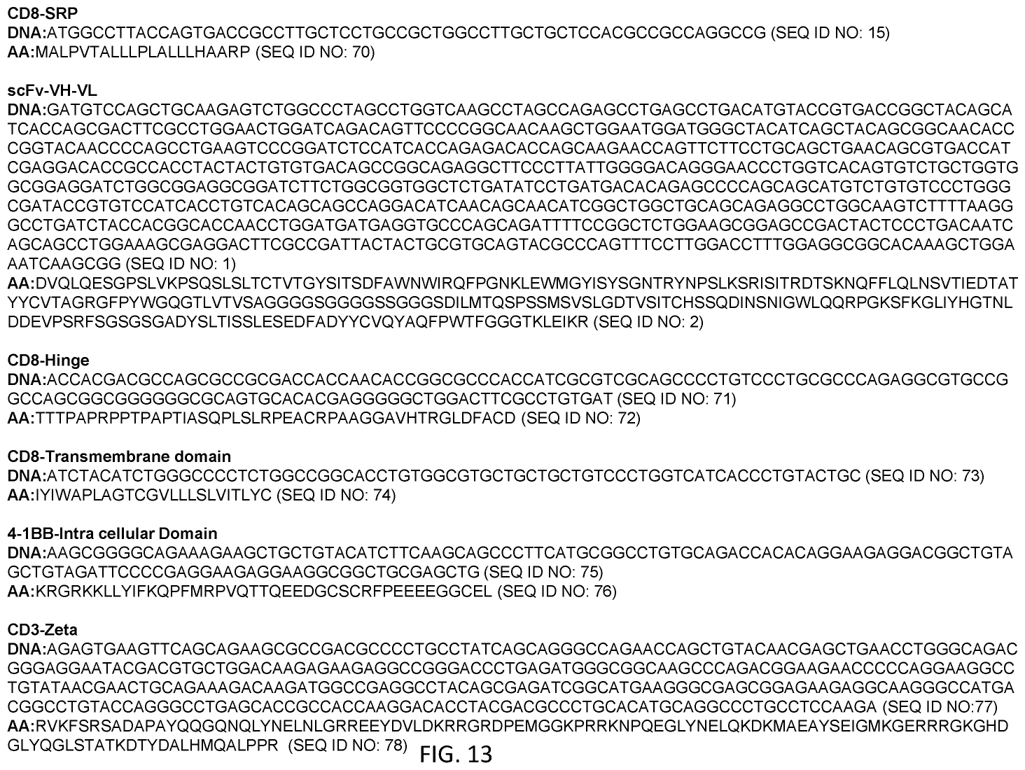

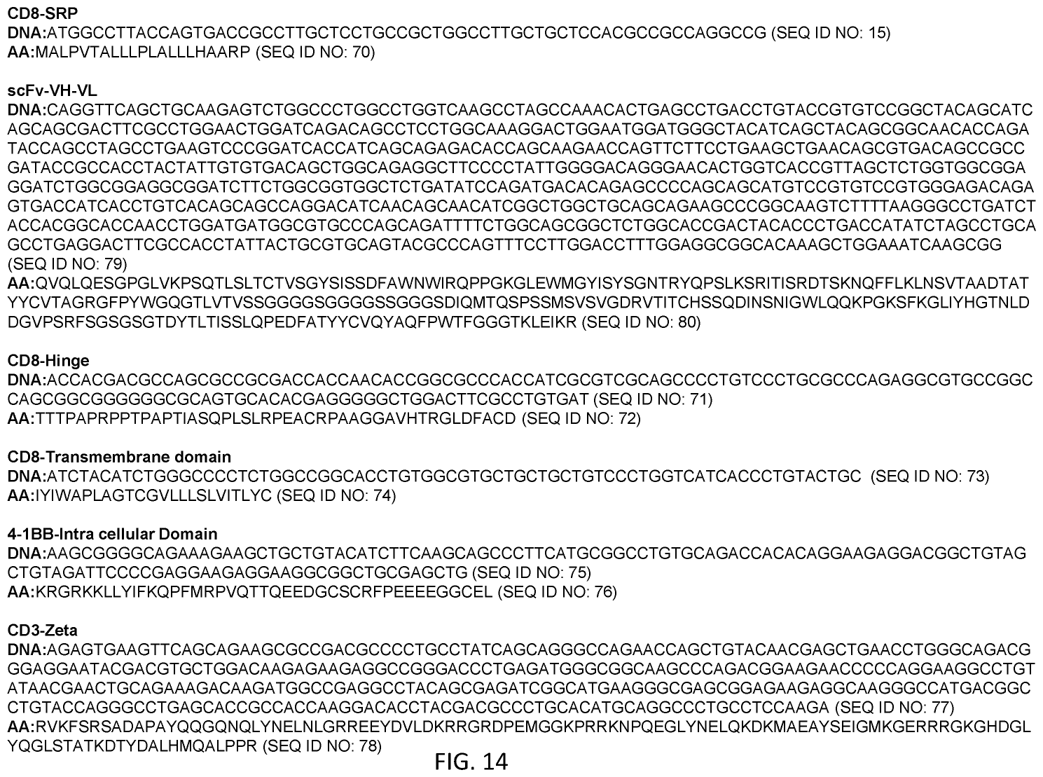

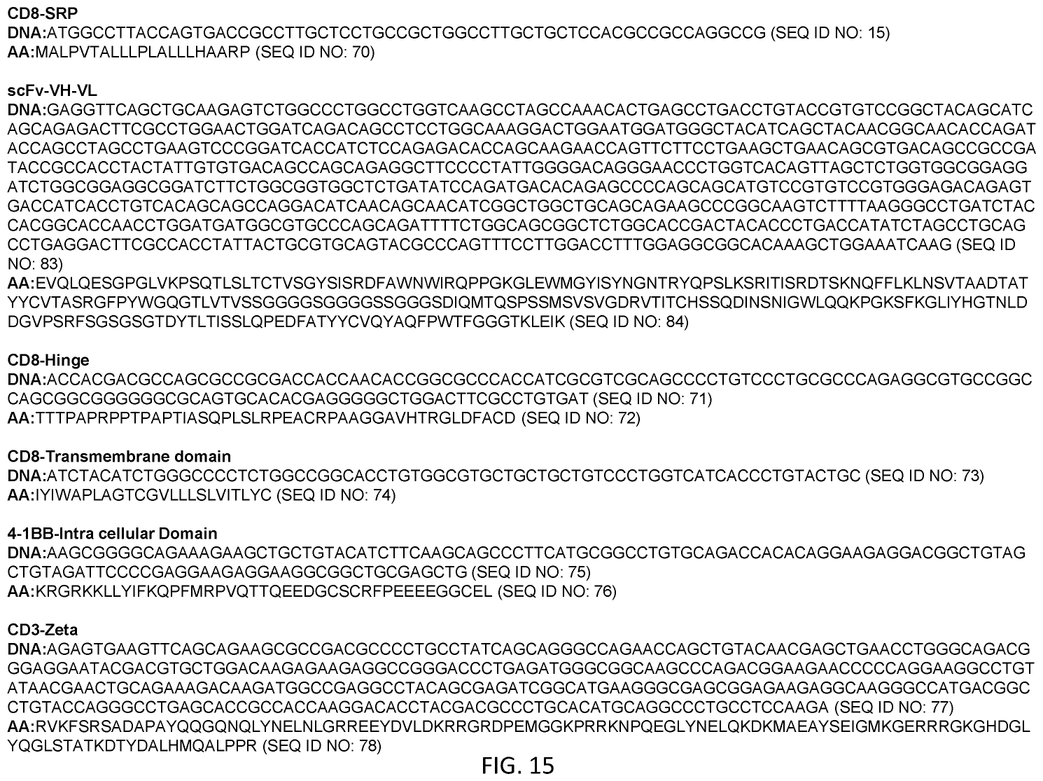

The sequences of the humanized form of mAb806, ABT-806, are shown in FIG. 11 (DNA) and FIG. 12 (amino acid).

### Example 3

### Targeting of EGFR-Specific Cells by 806 KIR CART Cells

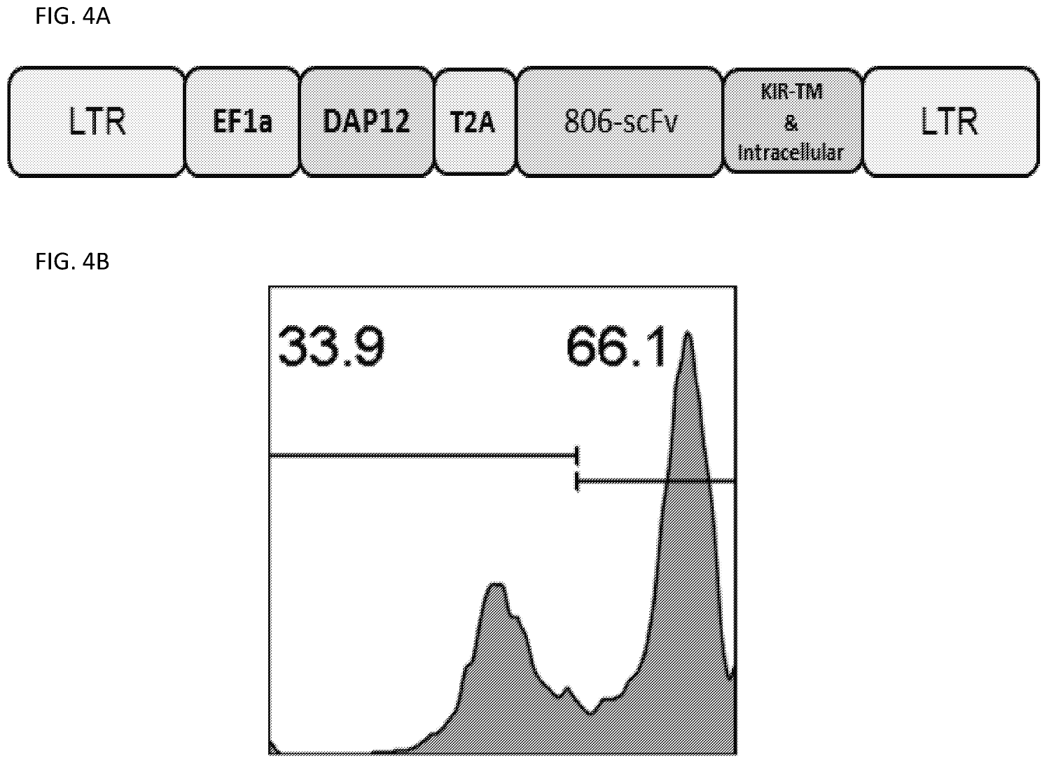

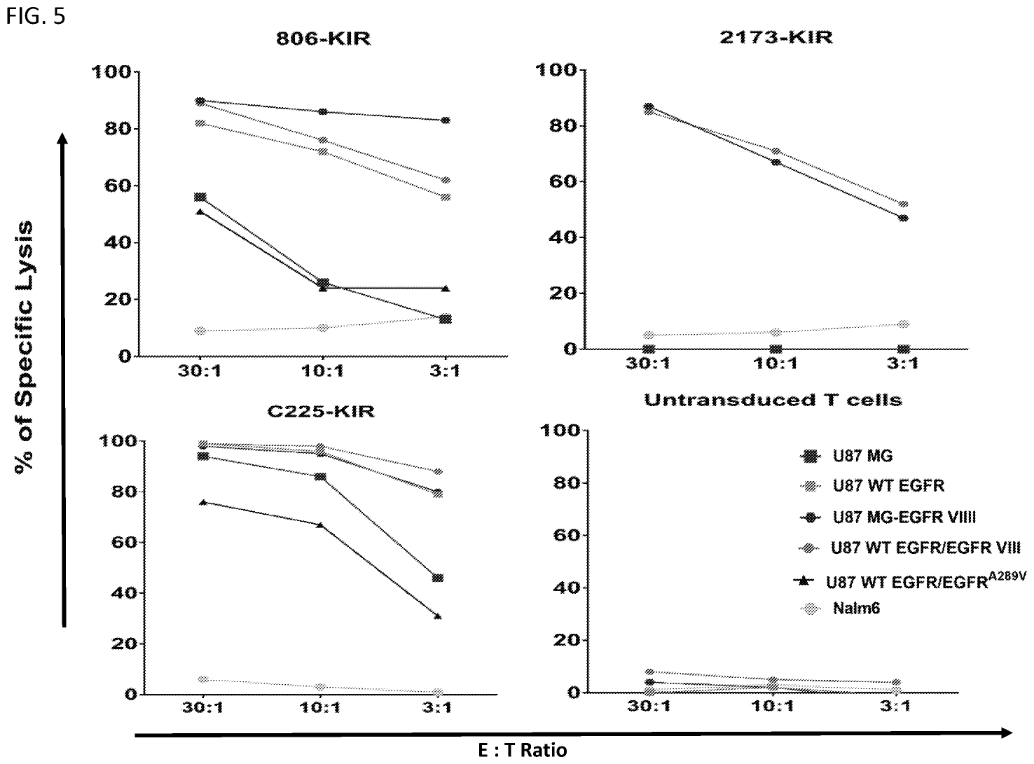

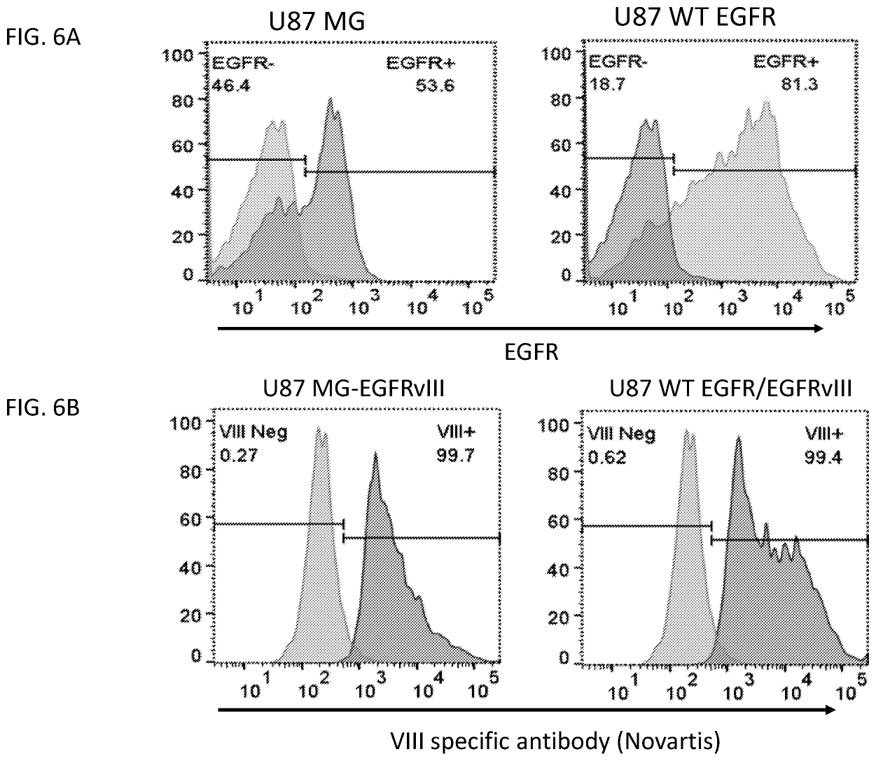

A KIR CAR that elicits broad specificity to multiple EGFR isoforms was also generated herein. A lentiviral vector was constructed that contained the 806-scFv, KIR transmembrane and intracellular domains, and a DAP12 sequence (FIG. 4A). Primary human T cells were simulated for 24 hours with anti-CD3/anti-CD28 T-cell activating beads. T cells were then transduced with the 806-KIR lentiviral vector and were expanded for 10 days in vitro. Approximately 44% of T cells expressed the 806 KIR CAR as analyzed by flow cytometry using biotinylated goat-anti-mouse F(ab)2 followed by streptavidin-APC (FIG. 4B). Antigen specific cytolytic activity of 806-KIR CAR T against EGFR- and its variants vIII- and A289V-expressing GBM cell lines was measured by using luciferase as a reporter gene for live cells (FIG. 5). 806 KIR CAR T cells were able to lyse U87 MG, U87 MG EGFRvIII, U87 wtEGFR, U87 wtEGFR/EGFRvIII, and U87 wtEGFR/EGFR^{A289V} cell lines. EGFRvIII specific 2173 and Cetuximab (C225) CARs, which recognize EGFRvIII and EGFR wild type, were used as positive controls. Data demonstrated antigen specific cytolytic activity of the 806 KIR CAR T cells.

### Example 4

### Targeted Cell Lysis of GBM Cross-Reactive EGFR-Specific Humanized CAR T Cells

A lentiviral expression vector encoding the humanized ABT806 scFv in a 4-1BBz CAR and a separate vector encoding the humanized ABT806 scFv in a KIR-CAR were created. The humanized sequences correspond to SEQ ID NOs. 23-28. Primary human CD4+ and CD8+ T cells were transduced with the vectors, demonstrating significant positivity after 6 days incubation. The humanized monoclonal ABT806 4-1BBz CAR and the humanized monoclonal ABT806 KIR CAR both demonstrated cytolytic activity against U87 MG cell lines modified with EGFR mutants or with U87 wtEGFR cell lines modified with EGFR mutants.

### Example 5

### Combination Treatments with Humanized 806-41BB CARs

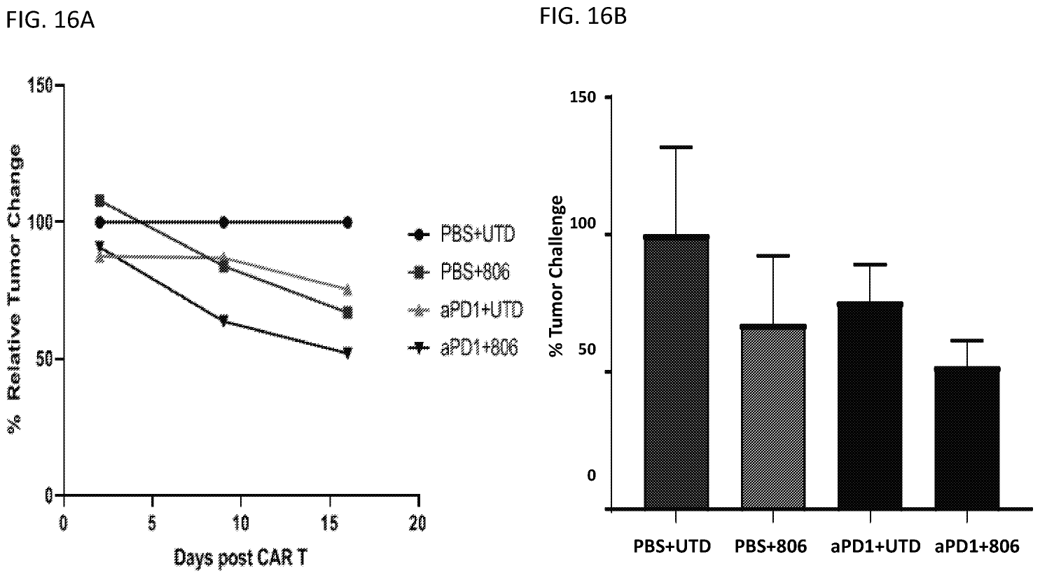

Subcutaneous animal combination experiments were performed using humanized 806-41BBz CARs and anti-PD-1 inhibitors tested against U87 wtEGFR/EGFRvIII (FIGS. 16A-16B). The dosing regimen of both anti-PD-1 inhibition and the CAR T cells is compared using multiple infusions to achieve the best clinical efficacy. Orthotopic tumor models are used for additional tumor growth inhibition studies of 806-41BB CAR T cells. Routes of delivery are compared using intravenous and intrathecal administration of 806 41BBz and 806 KIR CARs.

The scFv of Pembrolizumab, Nivolumab, and Atezolizumab are used to generate two versions of PD-1/PD-L1 blocker, resulting in 6 constructs, a minibody of each and a scFv in cis with the signaling domain of IFNγ. Plasmids encoding “PD-1/PD-L1 blocker secreted 806BB” are transfected into 293T cells. Supernatant is collected 72 hours after transfection and used in PD-1/PD-L1 binding assay by direct ELISA.

Supernatant from PD-1/PD-L1 blocker secreted 806 BBz CAR T cells is collected and used in PD-1/PD-L1 binding assay by direct ELISA. CAR expression of the 806 BBz CAR is detected on the T cells transduced with the PD-1/PD-L1 blocker secreted 806 BBz CAR T cells.

PD-1/PD-L1 blocker secreted 806 BBz CAR T cells are co-cultured with U87 wtEGFR/EGFRvIII positive target cells. Cytokine secretion is assessed 16 hours after co-culture, by flow based intracellular cytokine staining.

After subcutaneous tumor implantation, UTD T cells, 806 BBz CAR T cells, blocker secreted 806 BBz CAR T cells or blocker secreted T cells are infused through the tail veil. Tumor size and BLI signaling demonstrates the increased tumor growth inhibiting activity of the blocker secreted 806 BBz CAR T cells.

### Example 6

### In Vivo and In Vitro Administration of CAR T Cell Combination Therapies Against Human GBM

Subcutaneous tumors were treated with combination therapy of 806 BBz CAR and anti-PD1 antibody (FIGS. 16A-16B). Subcutaneous U87 wtEGFR/EGFRvIII cell lines were treated with combinations of either PBS or anti-PD-1 antibody and untransduced T cells or 806 BBz CAR T cells. Combination therapy demonstrated a larger decrease in relative tumor change, as determined by bioluminescence (FIG. 16A). Percent tumor change, relative to PBS+ untransduced (UTD) cells, on Day 16 post-CAR T infusion is shown in FIG. 16B.

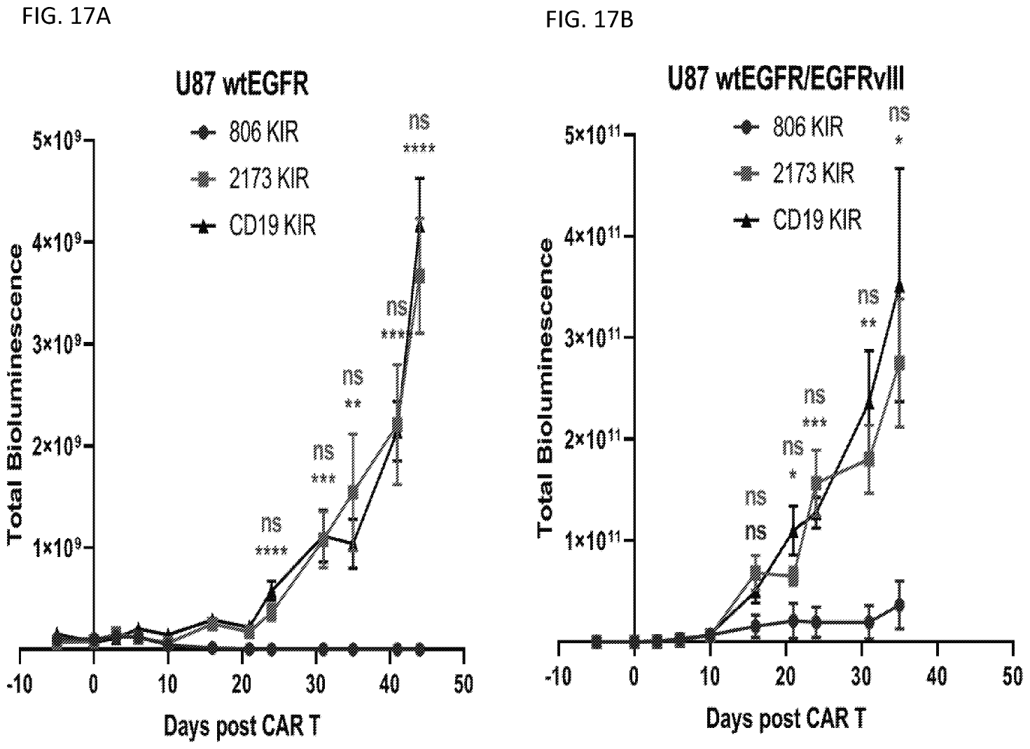

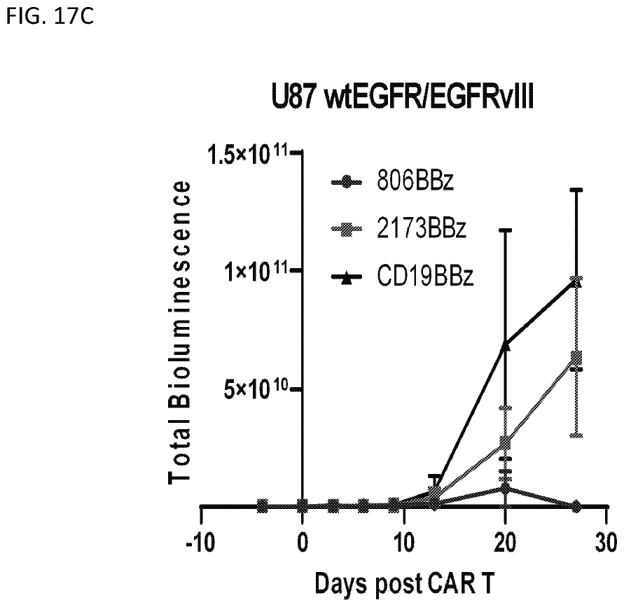

In vivo anti-tumor activity of the 806 KIR CAR was demonstrated against U87 wtEGFR (FIG. 17A) and U87 wtEGFR/EGFRvIII (FIG. 17B) flank tumors. Tumor models had overexpression of wildtype EGFR, either alone or in the presence of accompanying EGFR mutations. This pairing is a more physiologic representation than sole expression of the EGFR mutation, in the absence of overexpression of wildtype EGFR. In vivo anti-tumor activity of 806 BBz was also demonstrated against U87 wtEGFR/EGFRvIII flank tumors (FIG. 17C).

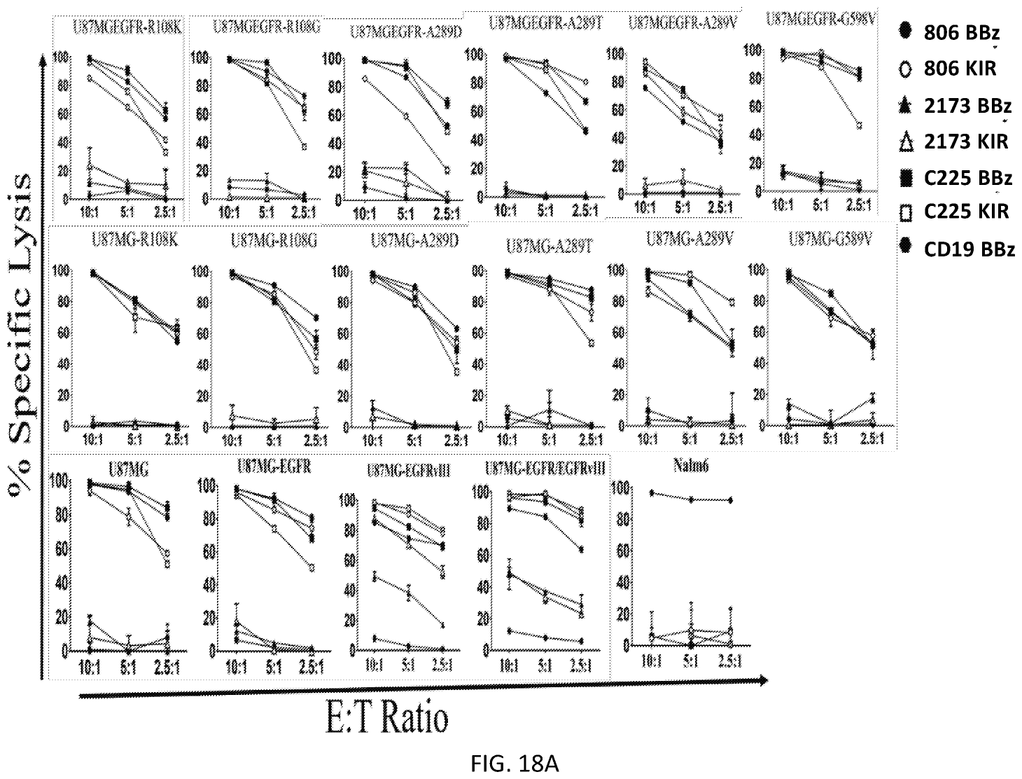

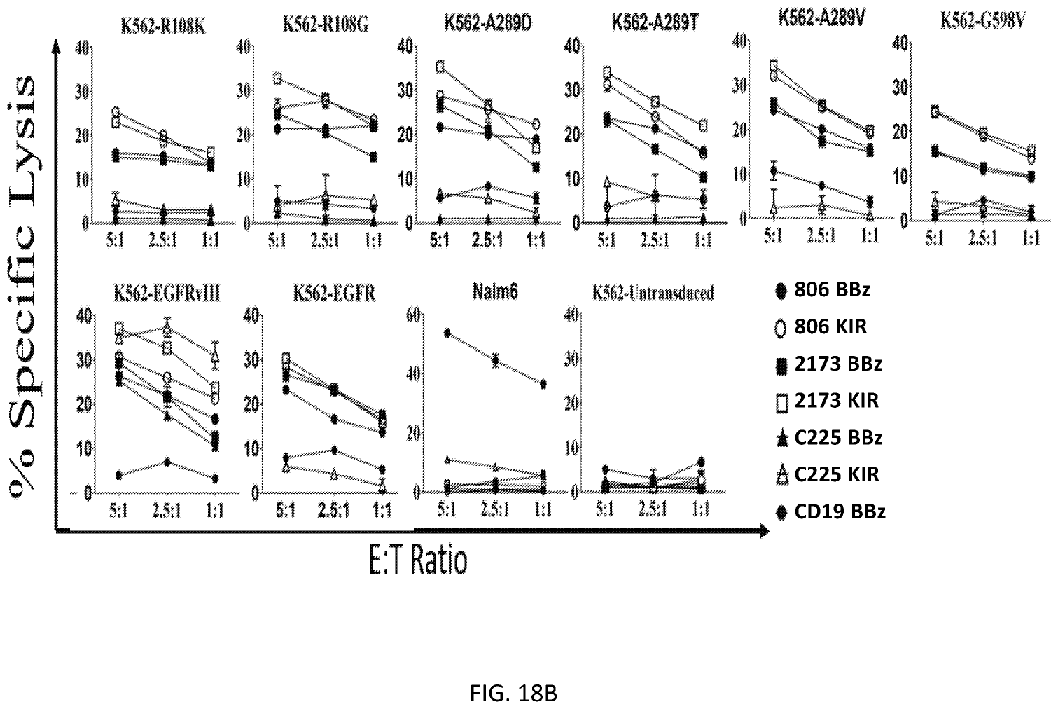

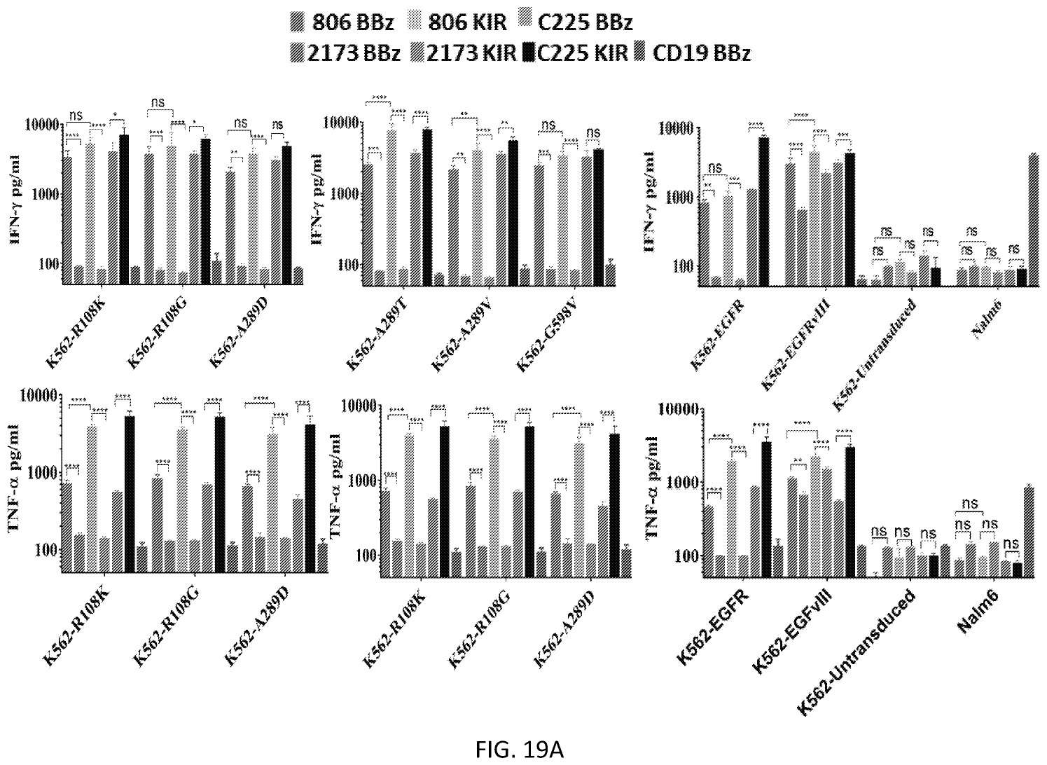

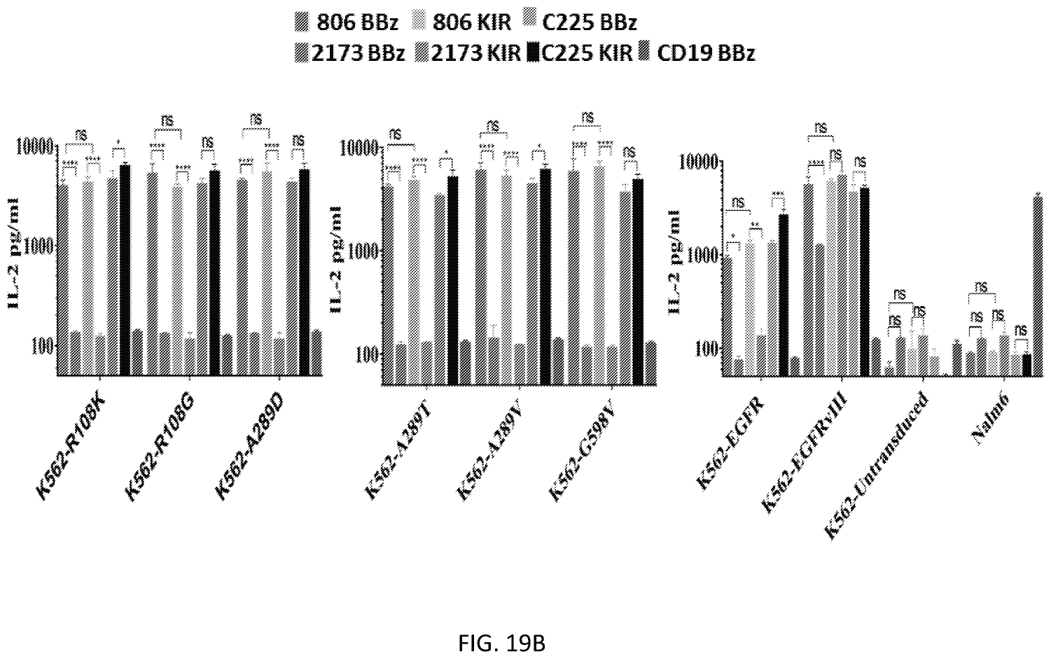

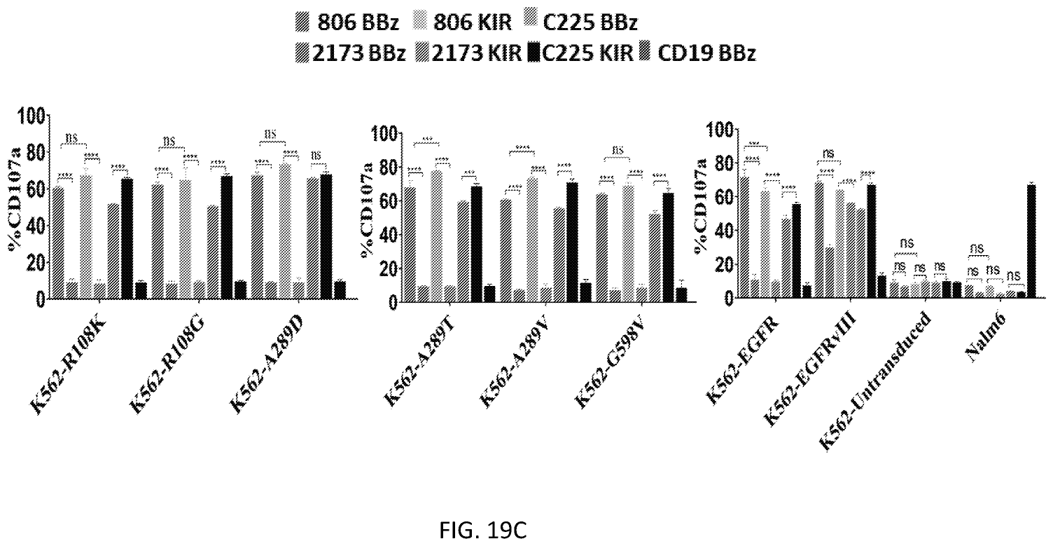

In vitro efficacy of 806 CART cells was also demonstrated (FIGS. 18A-18B and 19A-19C). Antigen specific cytolytic activity of 806 and 2173 CART cells in EGFR and its variants EGFRvIII, EGFR^{R108K/G} and EGFR^{A289D/T/V} expressing U87MG and U87 wtEGFR was shown in cell lines in 24 hour luciferase assay at indicated effector to target ratios (FIG. 18A). C225 BBz and C225 KIR CARS, which recognize wtEGFR, EGFRvIII, and its mutant variants, were used as positive controls and CD19 BBz CAR was used as a negative control. Antigen specific cytolytic activity of 806 and 2173 CAR T cells in EGFR and its variants expressing K562 cells was demonstrated in a 4 hour chromium release assay at indicated effector to target ratio (FIG. 18B). K562 cells express no basal EGFR, providing a clean background against which to test antigen specificity.

K562 cells expressing wtEGFR, EGFRvIII, or EGFR-mutants were co-cultured with 806 CAR T cells for 48 hours and IFN-γ, TNF-α and IL2 secretion was measured by ELISA (19A-19B). CD107a degranulation of CART cells when co-cultured for 4 hours with K562 cells expressing wtEGFR, EGFRvIII, or an EGFR mutant was measured. Results are presented as percentage of CD107a expression on CD3⁺cells (FIG. 19C).

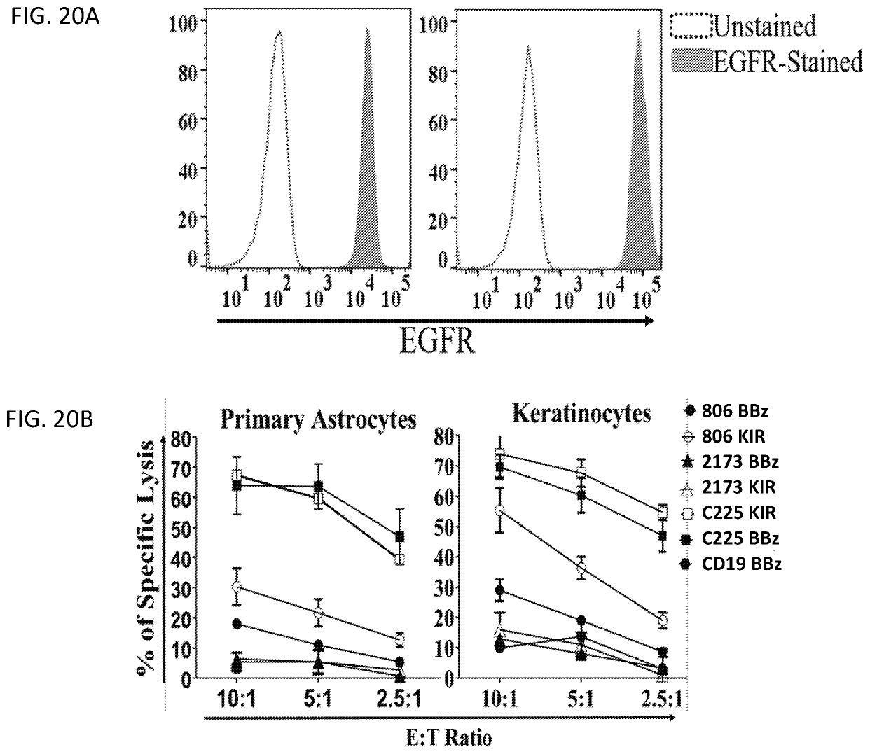

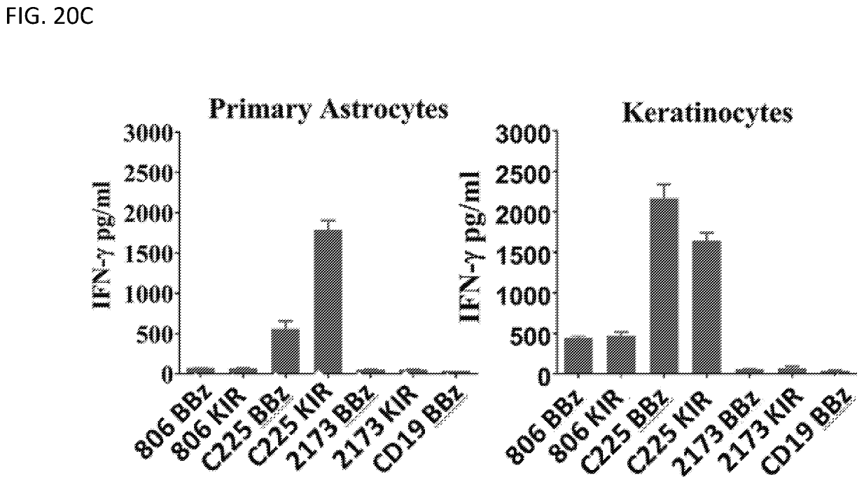

Anti-tumor efficacy of 806 CAR T cells was demonstrated in primary astrocytes and keratinocytes (FIGS. 20A-20C). Surface expression of EGFR was assessed by flow-cytometry on human primary astrocytes and keratinocytes (FIG. 20A). Primary astrocytes and keratinocytes were co-cultured with 806 CAR T cells at indicated ratios in a 4 hour chromium assay (FIG. 20B). Primary astrocytes and keratinocytes were co-cultured with 806 CAR T cells at effector to target ratio of 1:5 and IFN-γ was measured from supernatants after 24 hours incubation at 37° C. (FIG. 20C).

### Example 7

### Development of Advanced Biomarker Platform to Predict Clinical Efficacy of CAR T Treatment in Real Time

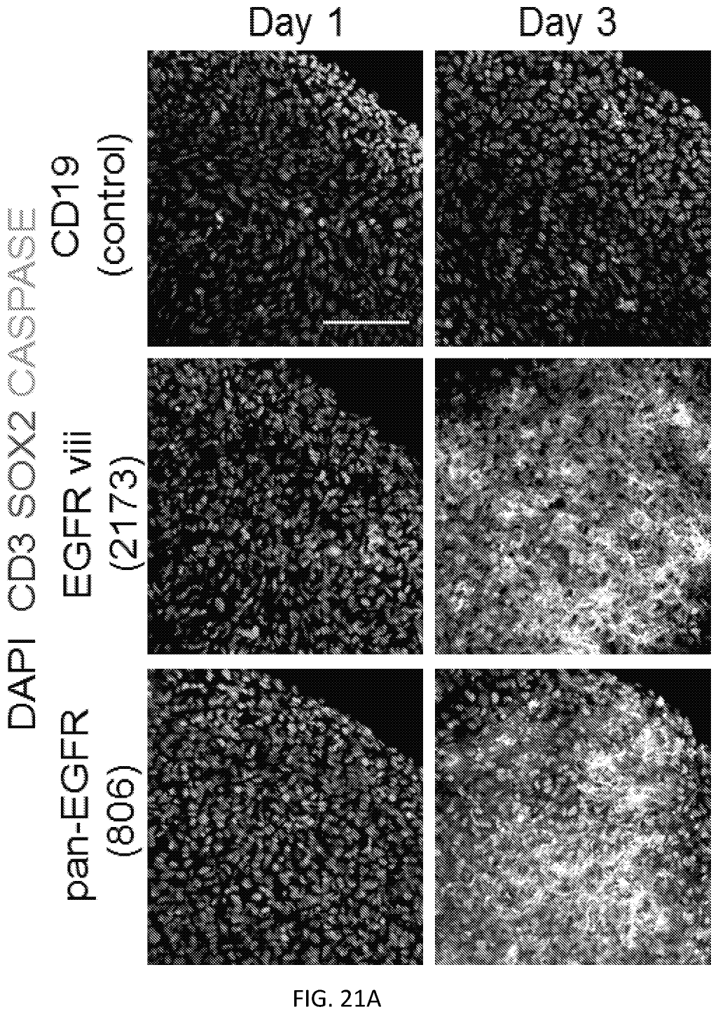

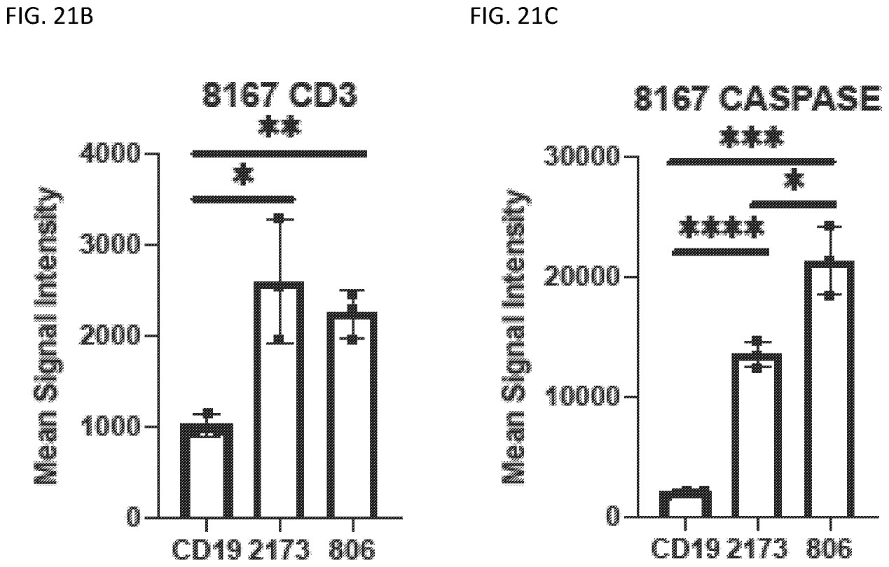

GBM organoid (GBO) was co-cultured with CAR T cells (FIGS. 21A-21C). GBOs are described in detail in Jacob et al. (2019) Cell 180:1;188-204.e22, contents of which are incorporated by reference in their entirety herein. Briefly, GBOs were generated by the following methods:

Glioblastoma tissue and peripheral blood samples were collected from patients with glioblastoma. Fresh surgically resected glioblastoma tissue was placed in sterile phosphate buffered saline and taken immediately to confirm a preliminary diagnosis of high-grade glioma by an attending neuropathologist . In cases where a large amount of en bloc tissue was available, the tissue was sub-divided into anatomically distinct subregions for analysis of intra-tumoral heterogeneity. After preliminary diagnosis of glioblastoma was confirmed, the tissue was distributed and placed in Hibernate A medium (BrainBits) kept at 4° C. For reliable organoid generation it was imperative that the tissue was processed immediately as a prolonged time between surgical removal and tissue processing reduced the reliability of GBO generation. The tissue was transferred to a sterile glass dish with H+GPSA medium containing Hibernate A, 1X GlutaMax (Thermo Fisher Scientific), 1X PenStrep (Thermo Fisher Scientific), and 1X Amphotericin B (Thermo Fisher Scientific) for dissection under a stereomicroscope (Zeiss) within a laminar flow biosafety cabinet. The amount of glioblastoma tissue received ranged from 0.5 to 2 mL in volume. The resected tumors were minced into approximately 0.5 to 1 mm diameter pieces using fine dissection scissors (Fine Science Tools) and washed with H+GPSA medium to remove cellular debris. Pieces containing substantial amounts of necrosis or surrounding brain tissue were removed. Tumor pieces were incubated in 1X RBC lysis buffer (Thermo Fisher Scientific) under gentle rotation for 10 minutes at room temperature to lyse the majority of contaminating red blood cells. RBC lysis buffer was aspirated, and tumor pieces were washed with H+GPSA medium. Several tumor pieces were snap frozen for bulk RNA sequencing and whole exome sequencing. For histological studies, several tumor pieces were placed directly in 4% methanol-free formaldehyde (Polysciences) diluted in DPBS (Thermo Fisher Scientific) for 1 hour at room temperature under gentle rotation. After fixation, the tumor pieces were placed in a plastic cryomold (Electron Microscopy Sciences) and snap frozen in tissue freezing medium (General Data) on dry ice. Frozen tissue was stored at −80° C. until processing.

The remaining tumor pieces not set aside for RNA sequencing, whole exome sequencing, or histology were distributed in ultra-low attachment 6-well culture plates (Corning) with 4 mL of GBO medium containing 50% DMEM:F12 (Thermo Fisher Scientific), 50% Neurobasal (Thermo Fisher Scientific), 1X GlutaMax (Thermo Fisher Scientific), 1X NEAAs (Thermo Fisher Scientific), 1X PenStrep (Thermo Fisher Scientific), 1X N2 supplement (Thermo Fisher Scientific), 1X B27 w/o vitamin A supplement (Thermo Fisher Scientific), 1X 2-mercaptoethanol (Thermo Fisher Scientific), and 2.5 mg/ml human insulin (Sigma) per well and placed on an orbital shaker rotating at 120 rpm within a 37° C., 5% CO2, and 90% humidity sterile incubator. Roughly 75% of the medium was changed every 48 hours by tilting the plates at a 45° angle and aspirating the medium above the sunken GBOs. Within the first week of culture, the tumor pieces often shed cellular and blood debris making the medium slightly cloudy. The shedding soon ceased, and the tumor pieces generally formed rounded organoids within 1-2 weeks, depending on tissue quality and patient-specific tumor growth characteristics. The criteria for successful establishment of GBOs from a given patient's tumor was that the micro-dissected tumor pieces survived for 2 weeks, developed a spherical morphology, and continuously grew in culture. GBOs cultured for prolonged periods of time (>1 month) were routinely cut to ˜200-500 mm diameter pieces using fine dissection scissors to prevent substantial necrosis within the center due to limited nutrient and oxygen diffusion. GBOs were sampled for RNA sequencing, whole exome sequencing, and histology.

Immunofluorescence staining of GBO was performed at 24 and 72 hours after co-culture with 806 BBZ, 2173 BBz, and CD19 BBz CAR T cells (FIG. 21A). Quantification of cell staining for CD3 and cleaved caspase 3 is shown in FIG. 21B and FIG. 21C, respectively. Although there was not a significant difference in CD3 expression, caspase activity was significantly different, demonstrating that 806 BBz CAR T cells led to increased tumor killing when compared to 2173 BBz CAR T cells. 8167 GBO expressed amplified endogenous wtEGFR, EGFRvIII, and EGFR^{A289V}, portraying a more physiologic representation of GBMs than standard glioma stem cell lines.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated nucleic acid molecule encoding a chimeric antigen receptor (CAR), wherein the CAR comprises an antigen binding domain capable of binding multiple isoforms of epidermal growth factor receptor (EGFR), a transmembrane domain, and an intracellular domain.

2. The isolated nucleic acid of claim 1, wherein the EGFR isoforms are selected from the group consisting of wild-type EGFR (wtEGFR), mutated EGFR, EGFR^{A289V}, EGFR^{A289D}, EGFR^{A289T}, EGFR^{R108K}, EGFR^{R108G}, EGFR^{G598V}, EGFR^{D126Y}, EGFR^{C628F}, EGFR^{R108K/A289V}, EGFR^{R108K/D126Y}, EGFRA^{A289V/G598V}, EGFR^{A289V/C628F}, and EGFR variant II.

3. The isolated nucleic acid of claim 1, wherein:
(a) the antigen binding domain is selected from the group consisting of an antibody, an scFv, a Fab, or any fragment thereof and/or
(b) the antigen binding domain is encoded by a nucleotide sequence selected from the group consisting of SEQ ID NO: 1, SEQ ID NO: 31, SEQ ID NO: 79, SEQ ID NO: 81, SEQ ID NO:83, and SEQ ID NO: 85; and/or
(c) the antigen binding domain comprises an amino acid sequence selected from the group consisting of SEQ ID NO: 2, SEQ ID NO: 32, SEQ ID NO: 80, SEQ ID NO: 82, SEQ ID NO: 84, and SEQ ID NO: 86; and/or
(d) the antigen binding domain comprises a light chain variable region comprising an amino acid sequence selected from the group consisting of SEQ ID NO: 3, SEQ ID NO: 27, and SEQ ID NO: 30; and/or
(e) the antigen binding domain comprises a heavy chain variable region comprising an amino acid sequence selected from the group consisting of SEQ ID NO: 4, SEQ ID NO: 26 and SEQ ID NO: 29; and/or
(f) the antigen binding domain comprises a light chain complementarity determining region (LCDR) comprising an amino acid sequence selected from the group consisting of SEQ ID NOs: 5, 6, and 7; and/or
(g) the antigen binding domain comprises a heavy chain complementarity determining region (HCDR) comprising an amino acid sequence selected from the group consisting of SEQ ID NOs: 8, 9, and 10.

4.-9. (canceled)

10. The isolated nucleic acid of claim 1, wherein:
(a) the CAR further comprises a hinge region; or
(b) the CAR further comprises a hinge region and the hinge region is encoded by the nucleotide sequence of SEQ ID NO: 11 or SEQ ID NO: 71.

11. (canceled)

12. The isolated nucleic acid of claim 1, wherein:
(a) the transmembrane domain is encoded by the nucleotide sequence of SEQ ID NO: 12 or SEQ ID NO: 73; and/or
(b) the intracellular domain is encoded by the nucleotide sequence of SEQ ID NO: 13 or SEQ ID NO: 75; and/or
(b) the intracellular domain is encoded by the nucleotide sequence comprising SEQ ID NO: 14 or SEQ ID NO: 77; and/or
(c) the intracellular domain is encoded by a nucleotide sequence comprising SEQ ID NO: 13 and SEQ ID NO: 14 or a nucleotide sequence comprising SEQ ID NO: 75 and SEQ ID NO: 77.

13.-15. (canceled)

16. The isolated nucleic acid of claim 1, wherein:
(a) the CAR is encoded by a nucleotide sequence selected from the group consisting of SEQ ID NOs: 21, 64, 66, or 68; or
(b) the CAR comprises an amino acid sequence selected from the group consisting of SEQ ID NOs: 22, 65, 67, and 69.

17. The isolated nucleic acid of claim 1, wherein:
(a) the transmembrane domain and/or the intracellular domain comprise a killer cell immunoglobulin-like receptor (KIR); and/or
(b) the transmembrane domain and/or the intracellular domain comprise a killer cell immunoglobulin-like receptor (KIR), and further comprising a nucleic acid encoding DAP12.

18.-19. (canceled)

20. The isolated nucleic acid of claim 1, wherein the CAR is capable of binding an EGFR homodimer, an EGFR heterodimer, an EGFR oligomer, and/or an EGFR/ErbB oligomer.

21. A vector comprising the isolated nucleic acid of claim 1.

22. A modified cell comprising a cross-reactive chimeric antigen receptor (CAR), wherein the CAR comprises an antigen binding domain capable of binding multiple isoforms of EGFR, a transmembrane domain, and an intracellular domain.

23. The modified cell of claim 22, wherein the EGFR isoforms are selected from the group consisting of wild-type EGFR (wtEGFR), mutated EGFR, EGFR^{A289V}, EGFR^{A289D}, EGFR^{A289T}, EGFR^{R108K}, EGFR^{R108G}, EGFR^{G598V}, EGFRD^{D126Y}, EGFR^{C628F}, EGFR^{R108K/A289V}, EGFR^{R108K/D126Y}. EGFR^{A289V/G598V}, EGFR^{A289V/C628F}, and EGFR variant II.

24. The modified cell of claim 22, wherein:
(a) the antigen binding domain is selected from the group consisting of an antibody, an scFv, a Fab, or any fragment thereof;
(b) the antigen binding domain is encoded by a nucleotide sequence selected from the group consisting of SEQ ID NO: 1, SEQ ID NO: 31, SEQ ID NO: 79, SEQ ID NO: 81, SEQ ID NO:83, and SEQ ID NO: 85; and/or
(c) the antigen binding domain comprises an amino acid sequence selected from the group consisting of SEQ ID NO: 2, SEQ ID NO: 32, SEQ ID NO: 80, SEQ ID NO: 82, SEQ ID NO: 84, and SEQ ID NO: 86; and/or
(d) the antigen binding domain comprises a light chain variable region comprising an amino acid sequence selected from the group consisting of SEQ ID NO: 3, SEQ ID NO: 27, and SEQ ID NO: 30; and/or
(e) the antigen binding domain comprises a heavy variable region comprising an amino acid sequence selected from the group consisting of SEQ ID NO: 4, SEQ ID NO: 26, and SEQ ID NO: 29; and/or
(f) the antigen binding domain comprises a light chain complementarity determining region (LCDR) comprising an amino acid sequence selected from the group consisting of SEQ ID NOs: 5, 6, and 7; and/or
(g) the antigen binding domain comprises a heavy chain complementarity determining region (HCDR) comprising an amino acid sequence selected from the group consisting of SEQ ID NOs: 8, 9, and 10.

25.-30. (canceled)

31. The modified cell of claim 22, wherein:
(a) the CAR further comprises a hinge region; and/or
(b) the CAR further comprises a hinge region and wherein the hinge region comprises the amino acid sequence of SEQ ID NO: 72.

32. (canceled)

33. The modified cell of claim 22, wherein:
(a) the transmembrane domain comprises the amino acid sequence of SEQ ID NO: 74; and/or
(b) the intracellular domain comprises the amino acid sequence of SEQ ID NO: 76; and/or
(c) the intracellular domain comprises the amino acid sequence of SEQ ID NO: 78; and/or
(d) the intracellular domain comprises the amino acid sequence of SEQ ID NO: 76 and SEQ ID NO: 78.

34.-36. (canceled)

37. The modified cell of claim 22, wherein:
(a) the CAR is encoded by a nucleotide sequence selected from the group consisting of SEQ ID NOs: 21, 64, 66, or 68; and/or
(b) the CAR comprises an amino acid sequence selected from the group consisting of SEQ ID NOs: 22, 65, 67, and 69.

38. The modified cell of claim 22, wherein:
(a) the transmembrane domain and/or the intracellular domain comprise a killer cell immunoglobulin-like receptor (KIR); and/or
(b) the transmembrane domain and/or the intracellular domain comprise a killer cell immunoglobulin-like receptor (KIR), and further comprising a nucleic acid encoding DAP12.

39.-40. (canceled)

41. The modified cell of claim 22, wherein the CAR is capable of binding an EGFR homodimer, an EGFR heterodimer, an EGFR oligomer, and/or an EGFR/ErbB oligomer.

42. The modified cell of claim 22, wherein:
(a) the cell is a T cell; and/or
(b) the cell is an autologous cell; and/or
(c) the cell is a human cell.

43.-44. (canceled)

45. A method for treating cancer in a subject in need thereof, the method comprising administering to the subject the modified cell of claim 22.

46. A method for treating cancer in a subject in need thereof, the method comprising administering to the subject a modified cell comprising a CAR, wherein the CAR comprises an antigen binding domain capable of binding multiple isoforms of EGFR, a transmembrane domain, and an intracellular domain.

47. The method of claim 46, wherein the EGFR isoforms are selected from the group consisting of wild-type EGFR (wtEGFR), mutated EGFR, EGFR^{A289V}, EGFR^{A289D}, EGFR^{A289T}, EGFR^{R108K}, EGFR^{R108G}, EGFR^{G598V}, EGFR^{D126Y}, EGFR^{C628F}, EGFR^{R108K/A289V}, EGFR^{R108K/D126Y}, EGFR^{A289V/G598V}, EGFR^{A289V/C628F}, and EGFR variant II.

48. The method of claim 46, wherein:
(a) the antigen binding domain is encoded by a nucleotide sequence selected from the group consisting of SEQ ID NO: 1, SEQ ID NO: 31, SEQ ID NO: 79, SEQ ID NO: 81, SEQ ID NO: 83, and SEQ ID NO: 85; and/or
(b) the antigen binding domain comprises an amino acid sequence selected from the group consisting of SEQ ID NO: 2, SEQ ID NO: 32, SEQ ID NO: 80, SEQ ID NO: 82, SEQ ID NO: 84, and SEQ ID NO: 86; and/or
(c) the antigen binding domain comprises a light chain variable region comprising an amino acid sequence selected from the group consisting of SEQ ID NO: 3, SEQ ID NO: 27, and SEQ ID NO: 30; and/or
(d) the antigen binding domain comprises a heavy variable region comprising an amino acid sequence selected from the group consisting of SEQ ID NO: 4, SEQ ID NO: 26, and SEQ ID NO: 29; and/or
(e) the antigen binding domain comprises a light chain complementarity determining region (LCDR) comprising an amino acid sequence selected from the group consisting of SEQ ID NOs: 5, 6, and 7; and/or
(f) the antigen binding domain comprises a heavy chain complementarity determining region (HCDR) comprising an amino acid sequence selected from the group consisting of SEQ ID NOs: 8, 9, and 10.

49.-53. (canceled)

54. The method of claim 46, wherein:
(a) the CAR is encoded by a nucleotide sequence selected from the group consisting of SEQ ID NOs: 21, 64, 66, or 68; or
(b) the CAR comprises an amino acid sequence selected from the group consisting of SEQ ID NOs: 22, 65, 67, and 69.

55. (canceled)

56. The method of claim 46, further comprising:
(a) administering an additional treatment to the subject; or
(b) administering an additional treatment to the subject, wherein the additional treatment comprises an immune checkpoint blockade (ICB); or
(c) administering an additional treatment to the subject, wherein the additional treatment comprises an ICB, wherein the ICB is selected from the group consisting of an anti-PD-I treatment, an anti-PD-LI treatment, an anti-TIM3 treatment, and an anti CTLA-4 treatment.

57.58. (canceled)

59. The method of claim 46, wherein:
(a) the treatment is delivered locally; and/or
(b) the modified cell further comprises a mini body; and/or
(c) the modified cell further comprises a mini body, and the mini body comprises an scFv specific for PD-I and a human IgG CH3 domain; and/or
(d) the modified cell further comprises a mini body, and the mini body comprises an scFv specific for CTLA-4 and a human IgG CH3 domain; and/or
(e) the modified cell further comprises a mini body, and the mini body comprises an scFv specific for TIM-3 and a human IgG CH3 domain; and/or
(f) the modified cell further comprises a mini body, the minibody comprises an scFv specific for PD-LI and a human IgG CH3 domain.

60.-64. (canceled)

65. A method of treating cancer in a subject in need thereof, the method comprising culturing a plurality of CART cells with a GBM organoid (GBO) derived from the subject, selecting from the plurality of CART cells, a CART cell having the highest efficacy, and administering the CART cell with the highest efficacy to the subject, thus treating the cancer in the subject.

66. The method of claim 65, wherein:
(a) the plurality of CART cells comprises a plurality of modified T cells comprising a plurality of CARs, wherein each CAR comprises an antigen binding domain, a transmembrane domain, and an intracellular domain; and/or
(b) the GBO is generated from a biopsy from the subject and/or
(c) the highest efficacy is measured as the highest degree of apoptosis and/or tumor cell killing; and/or
(d) the method further comprises administering an additional treatment to the subject and/or
(e) the method further comprises administering an additional treatment to the subject, wherein the additional treatment comprises an immune checkpoint blockade (ICB); and/or
(f) the method further comprises administering an additional treatment to the subject, wherein the additional treatment comprises an ICB, and wherein the ICB is selected from the group consisting of an anti-PD-I treatment, an anti-PD-LI treatment, an anti-TIM3 treatment, and an anti CTLA-4 treatment.

67. The method of claim 66, wherein the antigen binding domain is capable of binding an antigen selected from the group consisting of CD19, EGFR, multiple isoforms of EGFR (e.g. wild-type EGFR (wtEGFR), mutated EGFR, EGFR^{A289V}, EGFR^{A289D}, EGFR^{A289T}, EGFR^{R108K}, EGFR^{R108G}, EGFR^{G598V}, EGFR^{D126Y}, EGFR^{C628F}, EGFR^{R108K/A289V}, EGFR^{R108K/D126Y}, EGFR^{A289V/G598V}, EGFR^{A289V/C628F}, and EGFR variant II), PSMA, PSCA, and any tumor associated antigen (TAA).

68.-72. (canceled)


SEQUENCE LISTING (86 sequences)

SEQ ID NO: 1 - DNA, 717 bp, Artificial Sequence
  806 scFv
gatgtccagctgcaagagtctggccctagcctggtcaagcctagccagagcctgagcctgacatgtaccgtgaccggctacagcatcaccagcgacttcgcctggaactggatcagacagttccccggcaacaagctggaatggatgggctacatcagctacagcggcaacacccggtacaaccccagcctgaagtcccggatctccatcaccagagacaccagcaagaaccagttcttcctgcagctgaacagcgtgaccatcgaggacaccgccacctactactgtgtgacagccggcagaggcttcccttattggggacagggaaccctggtcacagtgtctgctggtggcggaggatctggcggaggcggatcttctggcggtggctctgatatcctgatgacacagagccccagcagcatgtctgtgtccctgggcgataccgtgtccatcacctgtcacagcagccaggacatcaacagcaacatcggctggctgcagcagaggcctggcaagtcttttaagggcctgatctaccacggcaccaacctggatgatgaggtgcccagcagattttccggctctggaagcggagccgactactccctgacaatcagcagcctggaaagcgaggacttcgccgattactactgcgtgcagtacgcccagtttccttggacctttggaggcggcacaaagctggaaatcaagcgg

SEQ ID NO: 2 - protein, 239 aa, Artificial Sequence
  806 scFv
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSSGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 3 - protein, 108 aa, Artificial Sequence
  806 VL
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 4 - protein, 116 aa, Artificial Sequence
  806 VH
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 5 - protein, 11 aa, Artificial Sequence
  LCDR1
HSSQDINSNIG

SEQ ID NO: 6 - protein, 7 aa, Artificial Sequence
  LCDR2
HGTNLDD

SEQ ID NO: 7 - protein, 9 aa, Artificial Sequence
  LCDR3
VQYAQFPWT

SEQ ID NO: 8 - protein, 11 aa, Artificial Sequence
  HCDR1
GYSITSDFAWN

SEQ ID NO: 9 - protein, 16 aa, Artificial Sequence
  HCDR2
GYISYSGNTRYNPSLK

SEQ ID NO: 10 - protein, 10 aa, Artificial Sequence
  HCDR3
VTAGRGFPYW

SEQ ID NO: 11 - DNA, 135 bp, Artificial Sequence
  CD8 hinge
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 12 - DNA, 72 bp, Artificial Sequence
  CD8 transmembrane
atctacatttgggcccctctggctggtacttgcggggtcctgctgctttcactcgtgatcactctttactgt

SEQ ID NO: 13 - DNA, 126 bp, Artificial Sequence
  41BB
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 14 - DNA, 336 bp, Artificial Sequence
  CD3zeta
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 15 - DNA, 63 bp, Artificial Sequence
  CD8 SRP
atggccttaccagtgaccgccttgctcctgccgctggccttgctgctccacgccgccaggccg

SEQ ID NO: 16 - DNA, 339 bp, Artificial Sequence
  DAP12
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 17 - DNA, 72 bp, Artificial Sequence
  T2A
gtcgagggcggcggagagggcagaggaagtcttctaacatgcggtgacgtggaggagaatcccggccctagg

SEQ ID NO: 18 - DNA, 258 bp, Artificial Sequence
  Linker KIRS2
ggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 19 - DNA, 1464 bp, Artificial Sequence
  806 BBZ CAR
atggccttaccagtgaccgccttgctcctgccgctggccttgctgctccacgccgccaggccgggatccgatgtccagctgcaagagtctggccctagcctggtcaagcctagccagagcctgagcctgacatgtaccgtgaccggctacagcatcaccagcgacttcgcctggaactggatcagacagttccccggcaacaagctggaatggatgggctacatcagctacagcggcaacacccggtacaaccccagcctgaagtcccggatctccatcaccagagacaccagcaagaaccagttcttcctgcagctgaacagcgtgaccatcgaggacaccgccacctactactgtgtgacagccggcagaggcttcccttattggggacagggaaccctggtcacagtgtctgctggtggcggaggatctggcggaggcggatcttctggcggtggctctgatatcctgatgacacagagccccagcagcatgtctgtgtccctgggcgataccgtgtccatcacctgtcacagcagccaggacatcaacagcaacatcggctggctgcagcagaggcctggcaagtcttttaagggcctgatctaccacggcaccaacctggatgatgaggtgcccagcagattttccggctctggaagcggagccgactactccctgacaatcagcagcctggaaagcgaggacttcgccgattactactgcgtgcagtacgcccagtttccttggacctttggaggcggcacaaagctggaaatcaagcgggctagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtga

SEQ ID NO: 20 - protein, 487 aa, Artificial Sequence
  806 BBZ CAR
MALPVTALLLPLALLLHAARPGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSSGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRASTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 1461 bp, Artificial Sequence
  806 KIR CAR
atggggggacttgaaccctgcagcaggttcctgctcctgcctctcctgctggctgtaagtggtctccgtcctgtccaggtccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaagtcgagggcggcggagagggcagaggaagtcttctaacatgcggtgacgtggaggagaatcccggccctaggatggccttaccagtgaccgccttgctcctgccgctggccttgctgctccacgccgccaggccgggatccgatgtccagctgcaagagtctggccctagcctggtcaagcctagccagagcctgagcctgacatgtaccgtgaccggctacagcatcaccagcgacttcgcctggaactggatcagacagttccccggcaacaagctggaatggatgggctacatcagctacagcggcaacacccggtacaaccccagcctgaagtcccggatctccatcaccagagacaccagcaagaaccagttcttcctgcagctgaacagcgtgaccatcgaggacaccgccacctactactgtgtgacagccggcagaggcttcccttattggggacagggaaccctggtcacagtgtctgctggtggcggaggatctggcggaggcggatcttctggcggtggctctgatatcctgatgacacagagccccagcagcatgtctgtgtccctgggcgataccgtgtccatcacctgtcacagcagccaggacatcaacagcaacatcggctggctgcagcagaggcctggcaagtcttttaagggcctgatctaccacggcaccaacctggatgatgaggtgcccagcagattttccggctctggaagcggagccgactactccctgacaatcagcagcctggaaagcgaggacttcgccgattactactgcgtgcagtacgcccagtttccttggacctttggaggcggcacaaagctggaaatcaagcgggctagcggtggcggaggttctggaggtgggggttcctcacccactgaaccaagctccaaaaccggtaaccccagacacctgcatgttctgattgggacctcagtggtcaaaatccctttcaccatcctcctcttctttctccttcatcgctggtgctccaacaaaaaaaatgctgctgtaatggaccaagagcctgcagggaacagaacagtgaacagcgaggattctgatgaacaagaccatcaggaggtgtcatacgcataa

SEQ ID NO: 22 - protein, 486 aa, Artificial Sequence
  806 KIR CAR
MGGLEPCSRFLLLPLLLAVSGLRPVQVQAQSDCSCSTVSPGVLAGIVMGDLVLTVLIALAVYFLGRLVPRGRGAAEAATRKQRITETESPYQELQGQRSDVYSDLNTQRPYYKVEGGGEGRGSLLTCGDVEENPGPRMALPVTALLLPLALLLHAARPGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSSGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRASGGGGSGGGGSSPTEPSSKTGNPRHLHVLIGTSVVKIPFTILLFFLLHRWCSNKKNAAVMDQEPAGNRTVNSEDSDEQDHQEVSYA

SEQ ID NO: 23 - DNA, 348 bp, Artificial Sequence
  Humanized 806 VH
caggttcagctgcaagagtctggccctggcctggtcaagcctagccaaacactgagcctgacctgtaccgtgtccggctacagcatcagcagcgacttcgcctggaactggatcagacagcctcctggcaaaggactggaatggatgggctacatcagctacagcggcaacaccagataccagcctagcctgaagtcccggatcaccatcagcagagacaccagcaagaaccagttcttcctgaagctgaacagcgtgacagccgccgataccgccacctactattgtgtgacagctggcagaggcttcccctattggggacagggaacactggtcaccgttagctct

SEQ ID NO: 24 - DNA, 324 bp, Artificial Sequence
  Humanized 806 VL
gatatccagatgacacagagccccagcagcatgtccgtgtccgtgggagacagagtgaccatcacctgtcacagcagccaggacatcaacagcaacatcggctggctgcagcagaagcccggcaagtcttttaagggcctgatctaccacggcaccaacctggatgatggcgtgcccagcagattttctggcagcggctctggcaccgactacaccctgaccatatctagcctgcagcctgaggacttcgccacctattactgcgtgcagtacgcccagtttccttggacctttggaggcggcacaaagctggaaatcaagcgg

SEQ ID NO: 25 - DNA, 672 bp, Artificial Sequence
  Humanized 806 scFv
caggttcagctgcaagagtctggccctggcctggtcaagcctagccaaacactgagcctgacctgtaccgtgtccggctacagcatcagcagcgacttcgcctggaactggatcagacagcctcctggcaaaggactggaatggatgggctacatcagctacagcggcaacaccagataccagcctagcctgaagtcccggatcaccatcagcagagacaccagcaagaaccagttcttcctgaagctgaacagcgtgacagccgccgataccgccacctactattgtgtgacagctggcagaggcttcccctattggggacagggaacactggtcaccgttagctctgatatccagatgacacagagccccagcagcatgtccgtgtccgtgggagacagagtgaccatcacctgtcacagcagccaggacatcaacagcaacatcggctggctgcagcagaagcccggcaagtcttttaagggcctgatctaccacggcaccaacctggatgatggcgtgcccagcagattttctggcagcggctctggcaccgactacaccctgaccatatctagcctgcagcctgaggacttcgccacctattactgcgtgcagtacgcccagtttccttggacctttggaggcggcacaaagctggaaatcaagcgg

SEQ ID NO: 26 - protein, 116 aa, Artificial Sequence
  Humanized 806 VH
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 27 - protein, 108 aa, Artificial Sequence
  Humanized 806 VL
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKR

SEQ ID NO: 28 - protein, 224 aa, Artificial Sequence
  Humanized 806 scFv
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSDIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKR

SEQ ID NO: 29 - protein, 116 aa, Artificial Sequence
  Affinity maturized 806 VH
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 30 - protein, 107 aa, Artificial Sequence
  Affinity maturized 806 VL
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 31 - protein, 717 aa, Artificial Sequence
  806 scFv
GATATTCTGATGACTCAATCTCCGTCTTCTATGAGCGTGAGCTTGGGTGACACCGTCAGCATCACCTGTCATTCCAGCCAGGATATAAACTCAAATATCGGCTGGCTCCAGCAACGCCCAGGCAAGTCATTCAAGGGGCTTATTTATCATGGCACCAATCTTGACGATGAAGTCCCATCACGCTTCAGCGGATCAGGCTCAGGTGCGGACTATTCCTTGACTATAAGTTCCCTCGAATCTGAGGATTTCGCCGACTATTATTGCGTACAATACGCCCAGTTTCCCTGGACCTTCGGAGGCGGCACCAAATTGGAGATAAAAAGGGGTGGAGGAGGATCAGGCGGGGGTGGAAGCGGCGGAGGAGGCAGCGACGTACAACTGCAAGAATCCGGGCCGAGTTTGGTCAAGCCCTCTCAATCTCTTTCTCTCACTTGCACGGTCACCGGATACTCCATAACCAGCGATTTTGCGTGGAATTGGATTCGACAATTTCCAGGGAATAAATTGGAATGGATGGGATATATCAGTTATTCTGGTAATACCAGATACAACCCGTCATTGAAAAGTCGCATCTCTATAACACGAGACACTTCAAAGAATCAGTTCTTCCTTCAGCTCAATTCTGTAACCATCGAAGATACTGCTACTTATTACTGTGTAACGGCGGGTCGAGGATTCCCCTACTGGGGCCAGGGTACACTGGTTACTGTTTCCGCC

SEQ ID NO: 32 - protein, 239 aa, Artificial Sequence
  806 scFv
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRGGGGSGGGGSGGGGSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 33 - DNA, 324 bp, Artificial Sequence
  806 VL
gatatcctgatgacacagagccccagcagcatgtctgtgtccctgggcgataccgtgtccatcacctgtcacagcagccaggacatcaacagcaacatcggctggctgcagcagaggcctggcaagtcttttaagggcctgatctaccacggcaccaacctggatgatgaggtgcccagcagattttccggctctggaagcggagccgactactccctgacaatcagcagcctggaaagcgaggacttcgccgattactactgcgtgcagtacgcccagtttccttggacctttggaggcggcacaaagctggaaatcaagcgg

SEQ ID NO: 34 - DNA, 348 bp, Artificial Sequence
  806 VH
gatgtccagctgcaagagtctggccctagcctggtcaagcctagccagagcctgagcctgacatgtaccgtgaccggctacagcatcaccagcgacttcgcctggaactggatcagacagttccccggcaacaagctggaatggatgggctacatcagctacagcggcaacacccggtacaaccccagcctgaagtcccggatctccatcaccagagacaccagcaagaaccagttcttcctgcagctgaacagcgtgaccatcgaggacaccgccacctactactgtgtgacagccggcagaggcttcccttattggggacagggaaccctggtcacagtgtctgct

SEQ ID NO: 35 - protein, 7 aa, Artificial Sequence
  LCDR2
HGTNLDD

SEQ ID NO: 36 - protein, 5 aa, Artificial Sequence
  Linker
  REPEAT (1)..(5): repeat n times, where n represents an integer of at least 1
GSGGS

SEQ ID NO: 37 - protein, 4 aa, Artificial Sequence
  Linker
  REPEAT (1)..(4): repeat n times, where n represents an integer of at least 1
GGGS

SEQ ID NO: 38 - protein, 5 aa, Artificial Sequence
  Linker
  REPEAT (1)..(5): repeat n times, where n represents an integer of at least 1
GGGGS

SEQ ID NO: 39 - protein, 4 aa, Artificial Sequence
  Linker
GGSG

SEQ ID NO: 40 - protein, 5 aa, Artificial Sequence
  Linker
GGSGG

SEQ ID NO: 41 - protein, 5 aa, Artificial Sequence
  Linker
GSGSG

SEQ ID NO: 42 - protein, 5 aa, Artificial Sequence
  Linker
GSGGG

SEQ ID NO: 43 - protein, 5 aa, Artificial Sequence
  Linker
GGGSG

SEQ ID NO: 44 - protein, 5 aa, Artificial Sequence
  Linker
GSSSG

SEQ ID NO: 45 - protein, 5 aa, Artificial Sequence
  Linker
GGGGS

SEQ ID NO: 46 - protein, 15 aa, Artificial Sequence
  Linker
GGGGSGGGGSGGGGS

SEQ ID NO: 47 - DNA, 45 bp, Artificial Sequence
  Linker
ggtggcggtggctcgggcggtggtgggtcgggtggcggcggatct

SEQ ID NO: 48 - protein, 15 aa, Artificial Sequence
  Linker
GGGGSGGGGSSGGGS

SEQ ID NO: 49 - DNA, 45 bp, Artificial Sequence
  Linker
ggtggcggaggatctggcggaggcggatcttctggcggtggctct

SEQ ID NO: 50 - protein, 5 aa, Artificial Sequence
  Hinge
DKTHT

SEQ ID NO: 51 - protein, 4 aa, Artificial Sequence
  Hinge
CPPC

SEQ ID NO: 52 - protein, 15 aa, Artificial Sequence
  Hinge
CPEPKSCDTPPPCPR

SEQ ID NO: 53 - protein, 12 aa, Artificial Sequence
  Hinge
ELKTPLGDTTHT

SEQ ID NO: 54 - protein, 10 aa, Artificial Sequence
  Hinge
KSCDKTHTCP

SEQ ID NO: 55 - protein, 7 aa, Artificial Sequence
  Hinge
KCCVDCP

SEQ ID NO: 56 - protein, 7 aa, Artificial Sequence
  Hinge
KYGPPCP

SEQ ID NO: 57 - protein, 15 aa, Artificial Sequence
  Hinge
EPKSCDKTHTCPPCP

SEQ ID NO: 58 - protein, 12 aa, Artificial Sequence
  Hinge
ERKCCVECPPCP

SEQ ID NO: 59 - protein, 17 aa, Artificial Sequence
  Hinge
ELKTPLGDTTHTCPRCP

SEQ ID NO: 60 - protein, 12 aa, Artificial Sequence
  Hinge
SPNMVPHAHHAQ

SEQ ID NO: 61 - protein, 15 aa, Artificial Sequence
  Hinge
EPKSCDKTYTCPPCP

SEQ ID NO: 62 - DNA, 348 bp, Artificial Sequence
  Affinity maturated humanized 806 VH
gaggttcagctgcaagagtctggccctggcctggtcaagcctagccaaacactgagcctgacctgtaccgtgtccggctacagcatcagcagagacttcgcctggaactggatcagacagcctcctggcaaaggactggaatggatgggctacatcagctacaacggcaacaccagataccagcctagcctgaagtcccggatcaccatctccagagacaccagcaagaaccagttcttcctgaagctgaacagcgtgacagccgccgataccgccacctactattgtgtgacagccagcagaggcttcccctattggggacagggaaccctggtcacagttagctct

SEQ ID NO: 63 - protein, 321 aa, Artificial Sequence
  Affinity maturated humanized 806 VL
GATATCCAGATGACACAGAGCCCCAGCAGCATGTCCGTGTCCGTGGGAGACAGAGTGACCATCACCTGTCACAGCAGCCAGGACATCAACAGCAACATCGGCTGGCTGCAGCAGAAGCCCGGCAAGTCTTTTAAGGGCCTGATCTACCACGGCACCAACCTGGATGATGGCGTGCCCAGCAGATTTTCTGGCAGCGGCTCTGGCACCGACTACACCCTGACCATATCTAGCCTGCAGCCTGAGGACTTCGCCACCTATTACTGCGTGCAGTACGCCCAGTTTCCTTGGACCTTTGGAGGCGGCACAAAGCTGGAAATCAAG

SEQ ID NO: 64 - DNA, 1449 bp, Artificial Sequence
  806 CAR
atggccttaccagtgaccgccttgctcctgccgctggccttgctgctccacgccgccaggccggatgtccagctgcaagagtctggccctagcctggtcaagcctagccagagcctgagcctgacatgtaccgtgaccggctacagcatcaccagcgacttcgcctggaactggatcagacagttccccggcaacaagctggaatggatgggctacatcagctacagcggcaacacccggtacaaccccagcctgaagtcccggatctccatcaccagagacaccagcaagaaccagttcttcctgcagctgaacagcgtgaccatcgaggacaccgccacctactactgtgtgacagccggcagaggcttcccttattggggacagggaaccctggtcacagtgtctgctggtggcggaggatctggcggaggcggatcttctggcggtggctctgatatcctgatgacacagagccccagcagcatgtctgtgtccctgggcgataccgtgtccatcacctgtcacagcagccaggacatcaacagcaacatcggctggctgcagcagaggcctggcaagtcttttaagggcctgatctaccacggcaccaacctggatgatgaggtgcccagcagattttccggctctggaagcggagccgactactccctgacaatcagcagcctggaaagcgaggacttcgccgattactactgcgtgcagtacgcccagtttccttggacctttggaggcggcacaaagctggaaatcaagcggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaaga

SEQ ID NO: 65 - protein, 483 aa, Artificial Sequence
  806 CAR
MALPVTALLLPLALLLHAARPDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSAGGGGSGGGGSSGGGSDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - DNA, 1449 bp, Artificial Sequence
  Humanized 806 CAR
atggccttaccagtgaccgccttgctcctgccgctggccttgctgctccacgccgccaggccgcaggttcagctgcaagagtctggccctggcctggtcaagcctagccaaacactgagcctgacctgtaccgtgtccggctacagcatcagcagcgacttcgcctggaactggatcagacagcctcctggcaaaggactggaatggatgggctacatcagctacagcggcaacaccagataccagcctagcctgaagtcccggatcaccatcagcagagacaccagcaagaaccagttcttcctgaagctgaacagcgtgacagccgccgataccgccacctactattgtgtgacagctggcagaggcttcccctattggggacagggaacactggtcaccgttagctctggtggcggaggatctggcggaggcggatcttctggcggtggctctgatatccagatgacacagagccccagcagcatgtccgtgtccgtgggagacagagtgaccatcacctgtcacagcagccaggacatcaacagcaacatcggctggctgcagcagaagcccggcaagtcttttaagggcctgatctaccacggcaccaacctggatgatggcgtgcccagcagattttctggcagcggctctggcaccgactacaccctgaccatatctagcctgcagcctgaggacttcgccacctattactgcgtgcagtacgcccagtttccttggacctttggaggcggcacaaagctggaaatcaagcggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaaga

SEQ ID NO: 67 - protein, 483 aa, Artificial Sequence
  Humanized 806 CAR
MALPVTALLLPLALLLHAARPQVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSGGGGSGGGGSSGGGSDIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 1446 bp, Artificial Sequence
  Affinity maturated humanized 806 CAR
atggccttaccagtgaccgccttgctcctgccgctggccttgctgctccacgccgccaggccggaggttcagctgcaagagtctggccctggcctggtcaagcctagccaaacactgagcctgacctgtaccgtgtccggctacagcatcagcagagacttcgcctggaactggatcagacagcctcctggcaaaggactggaatggatgggctacatcagctacaacggcaacaccagataccagcctagcctgaagtcccggatcaccatctccagagacaccagcaagaaccagttcttcctgaagctgaacagcgtgacagccgccgataccgccacctactattgtgtgacagccagcagaggcttcccctattggggacagggaaccctggtcacagttagctctggtggcggaggatctggcggaggcggatcttctggcggtggctctgatatccagatgacacagagccccagcagcatgtccgtgtccgtgggagacagagtgaccatcacctgtcacagcagccaggacatcaacagcaacatcggctggctgcagcagaagcccggcaagtcttttaagggcctgatctaccacggcaccaacctggatgatggcgtgcccagcagattttctggcagcggctctggcaccgactacaccctgaccatatctagcctgcagcctgaggacttcgccacctattactgcgtgcagtacgcccagtttccttggacctttggaggcggcacaaagctggaaatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcccctctggccggcacctgtggcgtgctgctgctgtccctggtcatcaccctgtactgcaagcggggcagaaagaagctgctgtacatcttcaagcagcccttcatgcggcctgtgcagaccacacaggaagaggacggctgtagctgtagattccccgaggaagaggaaggcggctgcgagctgagagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaaga

SEQ ID NO: 69 - protein, 482 aa, Artificial Sequence
  Affinity maturated humanized 806 CAR
MALPVTALLLPLALLLHAARPEVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSSGGGGSGGGGSSGGGSDIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 21 aa, Artificial Sequence
  SRP
MALPVTALLLPLALLLHAARP

SEQ ID NO: 71 - DNA, 135 bp, Artificial Sequence
  CD8 hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 72 - protein, 45 aa, Artificial Sequence
  CD8 hinge
TTTPAPRPPTPAPTIASQPLSLRPEACRPAAGGAVHTRGLDFACD

SEQ ID NO: 73 - DNA, 72 bp, Artificial Sequence
  CD8 transmembrane
atctacatctgggcccctctggccggcacctgtggcgtgctgctgctgtccctggtcatcaccctgtactgc

SEQ ID NO: 74 - protein, 24 aa, Artificial Sequence
  CD8 transmembrane
IYIWAPLAGTCGVLLLSLVITLYC

SEQ ID NO: 75 - DNA, 126 bp, Artificial Sequence
  41BB
aagcggggcagaaagaagctgctgtacatcttcaagcagcccttcatgcggcctgtgcagaccacacaggaagaggacggctgtagctgtagattccccgaggaagaggaaggcggctgcgagctg

SEQ ID NO: 76 - protein, 42 aa, Artificial Sequence
  41BB
KRGRKKLLYIFKQPFMRPVQTTQEEDGCSCRFPEEEEGGCEL

SEQ ID NO: 77 - DNA, 336 bp, Artificial Sequence
  CD3zeta
agagtgaagttcagcagaagcgccgacgcccctgcctatcagcagggccagaaccagctgtacaacgagctgaacctgggcagacgggaggaatacgacgtgctggacaagagaagaggccgggaccctgagatgggcggcaagcccagacggaagaacccccaggaaggcctgtataacgaactgcagaaagacaagatggccgaggcctacagcgagatcggcatgaagggcgagcggagaagaggcaagggccatgacggcctgtaccagggcctgagcaccgccaccaaggacacctacgacgccctgcacatgcaggccctgcctccaaga

SEQ ID NO: 78 - protein, 112 aa, Artificial Sequence
  CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - DNA, 717 bp, Artificial Sequence
  Humanized 806 scFv
caggttcagctgcaagagtctggccctggcctggtcaagcctagccaaacactgagcctgacctgtaccgtgtccggctacagcatcagcagcgacttcgcctggaactggatcagacagcctcctggcaaaggactggaatggatgggctacatcagctacagcggcaacaccagataccagcctagcctgaagtcccggatcaccatcagcagagacaccagcaagaaccagttcttcctgaagctgaacagcgtgacagccgccgataccgccacctactattgtgtgacagctggcagaggcttcccctattggggacagggaacactggtcaccgttagctctggtggcggaggatctggcggaggcggatcttctggcggtggctctgatatccagatgacacagagccccagcagcatgtccgtgtccgtgggagacagagtgaccatcacctgtcacagcagccaggacatcaacagcaacatcggctggctgcagcagaagcccggcaagtcttttaagggcctgatctaccacggcaccaacctggatgatggcgtgcccagcagattttctggcagcggctctggcaccgactacaccctgaccatatctagcctgcagcctgaggacttcgccacctattactgcgtgcagtacgcccagtttccttggacctttggaggcggcacaaagctggaaatcaagcgg

SEQ ID NO: 80 - protein, 239 aa, Artificial Sequence
  Humanized 806 scFv
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSGGGGSGGGGSSGGGSDIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKR

SEQ ID NO: 81 - DNA, 717 bp, Artificial Sequence
  Humanized 806 scFv
ggcgaactaaaggtcgaaacacggcggaggtttccaggttcctttgacccgcatgacgtgcgtcattatccaccgcttcaggagtccgacgtccgatctataccagtcccacatcagccacggtctcggcgacggtcttttagacgacccgtgcggtagtaggtccaaccacggcaccatctagtccgggaattttctgaacggcccgaagacgacgtcggtcggctacaacgacaactacaggaccgacgacactgtccactaccagtgagacagagggtgcctgtgcctgtacgacgaccccgagacacagtagacctatagtctcggtggcggtcttctaggcggaggcggtctaggaggcggtggtctcgattgccactggtcacaagggacaggggttatccccttcggagacggtcgacagtgtgttatcatccaccgccatagccgccgacagtgcgacaagtcgaagtccttcttgaccaagaacgaccacagagacgactaccactaggccctgaagtccgatccgaccatagaccacaacggcgacatcgactacatcgggtaggtaaggtcaggaaacggtcctccgacagactaggtcaaggtccgcttcagcgacgactacgacatcggcctgtgccatgtccagtccgagtcacaaaccgatccgaactggtccggtcccggtctgagaacgtcgacttggac

SEQ ID NO: 82 - protein, 239 aa, Artificial Sequence
  Humanized 806 scFv
RKIELKTGGGFTWPFQAYQVCYYTAFDEPQLSSITLTYDTGSGSGSFRSPVGDDLNTGHYILGKFSKGPKQQLWGINSNIDQSSHCTITVRDGVSVSMSSPSQTMQIDSGGGSSGGGGSGGGGSSVTVLTGQGWYPFGRGATVCYYTATDAATVSNLKLFFQNKSTDRSITIRSKLSPQYRTNGSYSIYGMWELGKGPPQRIWNWAFDSSISYGSVTCTLSLTQSPKVLGPGSEQLQVQ

SEQ ID NO: 83 - DNA, 714 bp, Artificial Sequence
  Affinity maturated humanized 806 scFv
gaggttcagctgcaagagtctggccctggcctggtcaagcctagccaaacactgagcctgacctgtaccgtgtccggctacagcatcagcagagacttcgcctggaactggatcagacagcctcctggcaaaggactggaatggatgggctacatcagctacaacggcaacaccagataccagcctagcctgaagtcccggatcaccatctccagagacaccagcaagaaccagttcttcctgaagctgaacagcgtgacagccgccgataccgccacctactattgtgtgacagccagcagaggcttcccctattggggacagggaaccctggtcacagttagctctggtggcggaggatctggcggaggcggatcttctggcggtggctctgatatccagatgacacagagccccagcagcatgtccgtgtccgtgggagacagagtgaccatcacctgtcacagcagccaggacatcaacagcaacatcggctggctgcagcagaagcccggcaagtcttttaagggcctgatctaccacggcaccaacctggatgatggcgtgcccagcagattttctggcagcggctctggcaccgactacaccctgaccatatctagcctgcagcctgaggacttcgccacctattactgcgtgcagtacgcccagtttccttggacctttggaggcggcacaaagctggaaatcaag

SEQ ID NO: 84 - protein, 238 aa, Artificial Sequence
  Affinity maturated humanized 806 scFv
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSSGGGGSGGGGSSGGGSDIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 85 - DNA, 714 bp, Artificial Sequence
  Affinity maturated humanized 806 scFv
gaactaaaggtcgaaacacggcggaggtttccaggttcctttgacccgcatgacgtgcgtcattatccaccgcttcaggagtccgacgtccgatctataccagtcccacatcagccacggtctcggcgacggtcttttagacgacccgtgcggtagtaggtccaaccacggcaccatctagtccgggaattttctgaacggcccgaagacgacgtcggtcggctacaacgacaactacaggaccgacgacactgtccactaccagtgagacagagggtgcctgtgcctgtacgacgaccccgagacacagtagacctatagtctcggtggcggtcttctaggcggaggcggtctaggaggcggtggtctcgattgacactggtcccaagggacaggggttatccccttcggagacgaccgacagtgtgttatcatccaccgccatagccgccgacagtgcgacaagtcgaagtccttcttgaccaagaacgaccacagagacctctaccactaggccctgaagtccgatccgaccatagaccacaacggcaacatcgactacatcgggtaggtaaggtcaggaaacggtcctccgacagactaggtcaaggtccgcttcagagacgactacgacatcggcctgtgccatgtccagtccgagtcacaaaccgatccgaactggtccggtcccggtctgagaacgtcgacttggag

SEQ ID NO: 86 - protein, 238 aa, Artificial Sequence
  Affinity maturated humanized 806 scFv
KIELKTGGGFTWPFQAYQVCYYTAFDEPQLSSITLTYDTGSGSGSFRSPVGDDLNTGHYILGKFSKGPKQQLWGINSNIDQSSHCTITVRDGVSVSMSSPSQTMQIDSGGGSSGGGGSGGGGSSVTVLTGQGWYPFGRSATVCYYTATDAATVSNLKLFFQNKSTDRSITIRSKLSPQYRTNGNYSIYGMWELGKGPPQRIWNWAFDRSISYGSVTCTLSLTQSPKVLGPGSEQLQVE